(19) United States
(12) Patent Application Publication
(10) Pub. No.: US 2021/0205417 A1
(43) Pub. Date: Jul. 8, 2021

(54) PH 7 INJECTABLE SOLUTION COMPRISING AT LEAST ONE BASAL INSULIN OF WHICH THE PI IS FROM 5.8 TO 8.5 AND A CO-POLYAMINO ACID BEARING CARBOXYLATE CHARGES AND HYDROPHOBIC RADICALS AND A LIMITED AMOUNT OF M-CRESOL

(71) Applicant: ADOCIA, Lyon (FR)

(72) Inventor: Alexandre GEISSLER, Lyon (FR)

(73) Assignee: ADOCIA, Lyon (FR)

(21) Appl. No.: 17/208,514

(22) Filed: Mar. 22, 2021

Related U.S. Application Data
(63) Continuation of application No. 16/213,963, filed on Dec. 7, 2018.

(51) Int. Cl. A61K 38/28; A61K 9/00; A61K 9/08; A61K 47/10; A61K 47/34

(52) U.S. Cl. CPC A61K 38/28; A61K 9/0019; A61K 47/34; A61K 47/10; A61K 9/08

(57) ABSTRACT

Physically stable compositions in the form of an injectable aqueous solution, the pH of which is from 6.0 to 8.0, comprising at least:
a) a basal insulin whose isoelectric point (pI) is from 5.8 to 8.5,
b) m-cresol in a concentration lower than or equal to 30 mM, and
c) a co-polyamino acid bearing carboxylate charges and at least one Formula X hydrophobic radical.

This is a Continuation of application Ser. No. 16/213,963 filed Dec. 7, 2018. The disclosure of the prior application is hereby incorporated by reference herein in its entirety.

The invention relates to insulin injection therapies for treating diabetes.

The invention relates to physically stable compositions in the form of an injectable aqueous solution, the pH of which is from 6.0 to 8.0, comprising at least one basal insulin whose isoelectric point (pI) is from 5.8 to 8.5 and a co-polyamino acid bearing carboxylate charges and hydrophobic radicals.

Insulin therapy, or diabetes therapy by insulin injection, has made remarkable progress in recent years, thanks to the development of new insulins that offer better correction of patients' blood glucose levels compared to human insulin, and that allow for better simulation of the physiological activity of the pancreas.

When type II diabetes is diagnosed in a patient, a treatment is implemented gradually. The patient first takes an oral antidiabetic drug (OAD) such as Metformin. When OADs alone are no longer sufficient to regulate blood glucose levels, a change in treatment must be made and, depending on the specificities of the patients, different combinations of treatments may be implemented. For example, the patient may be treated with a basal insulin of the insulin glargine or insulin detemir type in addition to OADs, and then, depending on the course of the pathology, treatment with basal and prandial insulin.

Moreover, today, to ensure the transition from OAD treatments, when they are no longer able to control blood glucose level, to a basal insulin/prandial insulin treatment, the injection of GLP-1 RA analogues is recommended.

GLP-1 RA for Glucagon-Like Peptide-1 receptor agonists, are insulinotropic or incretin peptides, and belong to the family of gastrointestinal hormones (or Gut Hormones) that stimulate insulin secretion when blood glucose levels are too high, for example after a meal.

Gut hormones are also called satiety hormones. These comprise GLP-1 RA (Glucagon-like peptide-1 receptor agonist) and GIP (Glucose-dependent insulinotropic peptide), oxyntomodulin (a derivative of proglucagon), peptide YY, amylin, cholecystokinin, pancreatic polypeptide (PP), ghrelin and enterostatin that have peptide or protein structures. They also stimulate the secretion of insulin, in response to glucose and fatty acids and are therefore potential candidates for the treatment of diabetes.

Among these, the GLP-1 RA are the ones that have produced the best results to date in the development of medications. They have made it possible for patients with type II diabetes to lose weight while having better control of their blood glucose.

GLP-1 RA analogues or derivatives thereof have thus been developed in particular to improve their stability.

On the other hand, to cover daily insulin requirements, two types of insulins with complementary actions are currently available to the diabetic patient: prandial insulins (or so-called rapid-acting insulins) and basal insulins. (or so-called slow-acting insulins).

Prandial insulins allow fast acting management (metabolization and/or storage) of glucose provided during meals and snacks. The patient should inject prandial insulin before each intake of food, i.e., about 2 to 3 injections per day. The most widely used prandial insulins are: recombinant human insulin, NovoLog® (insulin aspart from NOVO NORDISK), Humalog® (insulin lispro from ELI LILLY) and Apidra® (insulin glulisine from SANOFI).

Basal insulins maintain the glycemic homeostasis of the patient, outside of food intake periods. They essentially act to block the endogenous production of glucose (hepatic glucose). The daily dose of basal insulin is usually 40-50% of the total daily insulin requirement. Depending on the basal insulin used, this dose is given in 1 or 2 injections, distributed regularly throughout the day. The most commonly used basal insulins are Levemir® (insulin detemir from NOVO NORDISK) and Lantus® (insulin glargine from SANOFI).

It should be noted that NPH (NPH insulin for Neutral Protamine Hagedorn; Humulin NPH®, Insulatard®) is the oldest basal insulin. This formulation is the result of precipitation of human insulin (anionic at a neutral pH) by a cationic protein, protamine. The microcrystals thus formed are dispersed in an aqueous suspension and dissolve slowly after subcutaneous injection. This slow dissolution ensures a prolonged release of insulin. However, this release does not ensure a constant concentration of insulin over time. The release profile is bell-shaped and lasts only 12 to 16 hours. Therefore, it is injected twice a day. This NPH basal insulin is much less effective than the modern basal insulins, Levemir® and Lantus®. NPH is an intermediate-acting basal insulin.

The principle of NPH has evolved with the appearance of fast acting analogue insulins to give the products called “Premix” the ability to offer both fast action and intermediate action. NovoLog Mix® (NOVO NORDISK) and Humalog Mix® (ELI LILLY) are formulations comprising a fast acting analogue insulin, Novolog® and Humalog®, partially complexed with protamine. These formulations thus contain insulin-like microcrystals whose action is said to be intermediate and a part of insulin which remains soluble and whose action is rapid. These formulations offer the advantage of fast insulin, but they also have the defect of NPH, i.e., a limited duration of action of from 12 to 16 hours and insulin released in a “bell curve”. However, these products make it possible for the patient to receive an intermediate-acting basal insulin with a fast-acting prandial insulin through a single injection. Yet, many patients are concerned about reducing their number of injections.

Basal insulins currently on the market may be classified according to the technical solution that allows to obtain extended action and, presently, two approaches are used.

The first, that of insulin detemir, is the in vivo albumin bond. It is an analogue, soluble at pH 7, which comprises a fatty acid side chain (tetradecanoyl) attached to position B29 which, in vivo, allows this insulin to associate with albumin. Its prolonged action is mainly due to this affinity for albumin after a subcutaneous injection.

However its pharmacokinetic profile does not allow coverage for a full day, so it is most often used as two injections per day.

Another insulin soluble at pH 7 is degludec insulin sold under the name of Tresiba®^{d}. It also includes a fatty acid side chain attached to the insulin (hexadecanoyl-γ-L-Glu).

The second, that of insulin glargine, is the precipitation at the physiological pH. Insulin glargine is an analogue of human insulin obtained by elongation of the C-terminal part of the B-chain of human insulin by two arginine residues, and by substitution of the A21 asparagine residue with a glycine residue. (U.S. Pat. No. 5,656,722). The addition of two arginine residues was designed to adjust the pI (isoelectric point) of insulin glargine to physiological pH, and thus make this human insulin analogue insoluble in a physiological medium.

Also, the substitution of A21 was designed to make insulin glargine stable at acidic pH and thus be able to formulate it as an injectable solution at acidic pH. During subcutaneous injection, the passage of insulin glargine from an acidic pH (pH 4-4.5) to a physiological pH (neutral pH) causes its precipitation under the skin. The slow redissolution of the insulin glargine microparticles ensures a slow and prolonged action.

The hypoglycemic effect of insulin glargine is almost constant over a 24-hour period, which makes it possible for most patients to limit themselves to one injection per day.

Insulin glargine is considered today as the most used basal insulin.

However, the pH, which must be acidic, of the basal insulin formulations, whose isoelectric point is from 5.8 to 8.5, of the insulin glargine type, can present a real disadvantage, because the acidic pH of the insulin glargine formulation sometimes causes pain at injection in patients and, especially, prevents any formulation with other proteins and in particular with prandial insulins because they are not stable at acidic pH. The impossibility of formulating a prandial insulin, at acidic pH, relates to the fact that under these conditions, a prandial insulin undergoes a deamidation in position A21side reaction, which makes it impossible to meet the stability requirements applicable to injectable drugs.

To date, in applications WO 2013/021143 A1, WO 2013/104861 A1, WO 2014/124994 A1 and WO 2014/124993 A1 it was demonstrated that it is possible to solubilize these basal insulins, of the insulin glargine type, the isoelectric point of which is from 5.8 to 8.5, at neutral pH, while maintaining a difference in solubility between the in-vitro medium (the container) and the in-vivo medium (under the skin), regardless of the pH.

Application WO 2013/104861 A1, in particular, describes compositions in the form of an injectable aqueous solution, the pH of which is from 6.0 to 8.0, comprising at least (a) one basal insulin whose isoelectric point pI is from 5.8 to 8.5 and (b) a co-polyamino acid bearing carboxylate charges substituted by hydrophobic radicals.

These compositions from the prior art have the major disadvantage of not being sufficiently stable to meet the specifications applicable to pharmaceutical formulations.

Therefore, there is a need to find a solution that makes it possible to solubilize a basal insulin whose isoelectric point (pI) is from 5.8 to 8.5 while maintaining its basal profile after injection but which also makes it possible to satisfy standard physical stability conditions for insulin-based pharmaceuticals.

Surprisingly, the applicant has found that the co-polyamino acids bearing carboxylate charges and hydrophobic radicals according to the invention make it possible to obtain compositions in the form of solutions which not only meet the requirements described in WO 2013/104861 A1, but which moreover are able to confer an improvement to the physical stability of said compositions without having to increase the amount of excipients used.

Co-polyamino acids bearing carboxylate charges and hydrophobic radicals Hy according to the invention exhibit excellent resistance to hydrolysis. This can be verified specifically under accelerated conditions, for example through hydrolysis tests at basic pH (pH 12).

In addition, forced oxidation tests, for example of the Fenton oxidation type, show that the co-polyamino acids bearing carboxylate charges and hydrophobic radicals Hy exhibit good resistance to oxidation.

However, in the case of pharmaceutical compositions comprising basal insulin having a pI of from 5.8 to 8.5, and polymers bearing carboxylate charges and hydrophobic radicals, there is a need to improve both of the following aspects:
increase the duration of action of said basal insulin, and
reduce the amount of non-FAT excipients that may be used in these compositions.

The applicant has found conditions to improve the duration of action of said insulin basal and/or to reduce the amount of polymers bearing carboxylate charges and hydrophobic radicals.

The invention relates to physically stable compositions in the form of an injectable aqueous solution, the pH of which is from 6.0 to 8.0, comprising at least:
a) a basal insulin whose isoelectric point (pI) is from 5.8 to 8.5,
b) m-cresol in a concentration lower than or equal to 30 mM, and
b) a co-polyamino acid bearing carboxylate charges and at least one hydrophobic radical according to formula X.

In one embodiment, the invention concerns a composition in the form of an injectable aqueous solution, the pH of which is from 6.0 to 8.0, comprising at least:
a) a basal insulin whose isoelectric point pI is from 5.8 to 8.5;
b) m-cresol in a concentration lower than or equal to 30 mM, and
c) a co-polyamino acid bearing carboxylate charges and hydrophobic radicals -Hy, said co-polyamino acid consisting of glutamic or aspartic units and said hydrophobic radicals Hy being according to the following Formula X:

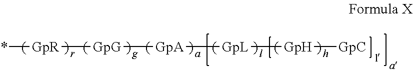

in which
GpR is chosen among the radicals according to formulas VII, VII′ or VII″:

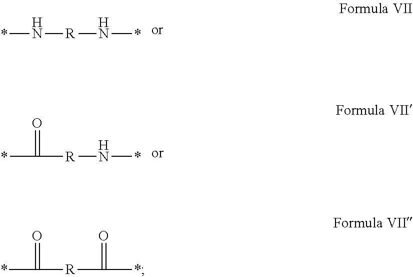

GpG and GpH, which are identical or different, are chosen among the radicals according to formulas XI or XI′:

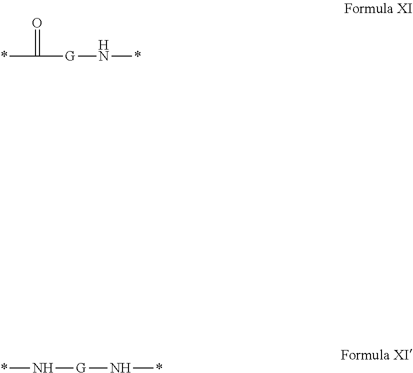

GpA is chosen among the radicals according to formula VIII

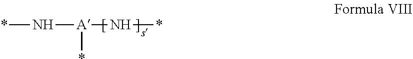

In which A′ is chosen among the radicals according to formulas VIII′, VIII″ or VIII′″

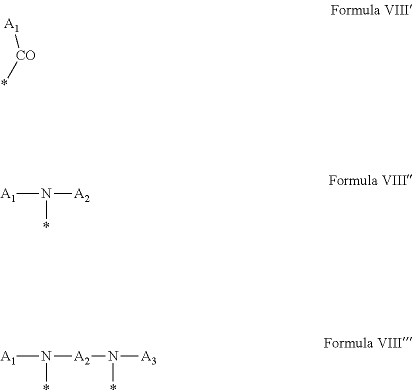

GpL is chosen among the radicals according to formula XII

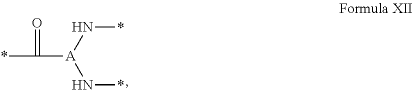

GpC is a radical according to formula IX:

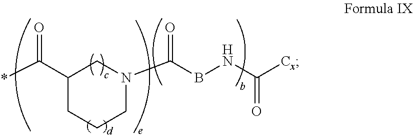

the * indicates the attachment sites of the different groups bound by amide functions;
a is an integer equal to 0 or 1 and a′=1 if a=0 and a′=1, 2 or 3 if a=1;
a′ is an integer equal to 1, to 2 or 3
b is an integer equal to 0 or 1;
c is an integer equal to 0 or 1, and if c is equal to 0 then d is equal to 1 or 2;
d is an integer equal to 0, 1 or 2;
e is an integer equal to 0 or 1;
g is an integer equal to 0, 1, 2, 3, 4, 5 or 6;
h is an integer equal to 0, 1, 2, 3, 4, 5 or 6,
l is an integer equal to 0 or 1 and l′=1 if l=0 and l′=2 if l=1;
r is an integer equal to 0, 1 or 2, and
s′ is an integer equal to 0 or 1, and
A, A₁, A₂ and A₃, which are identical or different, are linear or branched alkyl radicals, and optionally substituted by a radical from a saturated, unsaturated or aromatic ring, comprising from 1 to 8 carbon atoms;
B is a linear or branched alkyl radical, optionally comprising an aromatic ring comprising from 1 to 9 carbon atoms or an unsubstituted ether or polyether radical comprising from 4 to 14 carbon atoms and from 1 to 5 oxygen atoms;
Cₓ is a linear or branched monovalent alkyl radical, optionally comprising a cyclic part, in which x indicates the number of carbon atoms and:
   When the hydrophobic radical -Hy bears 1 -GpC, then 9≤x≤25,
   When the hydrophobic radical -Hy bears 2 -GpC, then 9≤x≤15,
   When the hydrophobic radical -Hy bears 3 -GpC, then 7≤x≤13,
   When the hydrophobic radical -Hy bears 4 -GpC, then 7≤x≤11,
   When the hydrophobic radical -Hy bears at least 5 -GpC, then 6≤x≤11,
G is a branched alkyl radical of 1 to 8 carbon atoms, said alkyl radical bearing one or more free carboxylic acid function(s).
R is a radical chosen in the group consisting of a linear or branched divalent alkyl radical comprising from 1 to 12 carbon atoms, a linear or branched divalent alkyl radical comprising from 1 to 12 carbon atoms bearing one or more —CONH₂ functions or an unsubstituted ether or polyether radical comprising from 4 to 14 carbon atoms and from 1 to 5 oxygen atoms:
the hydrophobic radical(s) -Hy according to formula X being bound to PLG:
   via a covalent bond between a carbonyl of the hydrophobic radical -Hy and a nitrogen atom borne by the PLG thus forming an amide function resulting from the reaction of an amine function borne by the PLG and an acid function borne by the precursor -Hy′ of the
   hydrophobic radical -Hy, and
   via a covalent bond between a nitrogen atom from the hydrophobic radical -Hy and a carbonyl borne by the PLG thus forming an amide function resulting from the reaction of an amine function of the precursor -Hy′ of the hydrophobic radical -Hy and an acid function borne by the PLG,
the ratio M between the number of hydrophobic radicals and the number of glutamic or aspartic units being between 0<M≤0.5;
when several hydrophobic radicals are borne by a co-polyamino acid then they are identical or different,
the degree of polymerization DP in glutamic or aspartic units for the PLG chains is from 5 to 250;
the free carboxylic acid functions being in the form of an alkali metal salt chosen in the group consisting of Na⁺ and K⁺.

The invention also relates to a method of preparing stable injectable compositions.

The pH of the compositions according to the invention is from 6.0 to 8.0, preferably from 6.6 to 7.8 or even more preferably from 6.8 to 7.6.

Said co-polyamino acid bearing carboxylate charges and hydrophobic radicals Hy is soluble in an aqueous solution at a pH of from 6.0 to 8.0, at a temperature of 25° C. and at a concentration of less than 100 mg/ml.

The co-polyamino acid is a statistical co-polyamino acid in the chain of glutamic and/or aspartic unit.

By “alkyl radical” is meant a linear or branched carbon chain, which does not comprise a heteroatom.

In the formulas, the * indicates the attachment sites of the various elements represented.

The term “physically stable composition” means compositions which satisfy the criteria of the visual inspection described in the European, American and International Pharmacopoeia, that is to say compositions which are clear, and which do not contain visible particles, but are also colorless.

By “Injectable aqueous solution” is meant solutions whose solvent is water, and which satisfies the conditions of the EP and US Pharmacopoeias.

The compositions in the form of an aqueous solution for injection according to the invention are clear solutions. By “clear solution” is meant compositions which satisfy the criteria described in the US and European Pharmacopoeias concerning injectable solutions. In the US Pharmacopoeia, the solutions are defined in part <1151> referring to the injection <1> (referring to <788> according to USP 35 and specified in <788> according to USP 35 and in <787>, <788> and <790> of USP 38 (as of Aug. 1, 2014), according to USP 38). In the European Pharmacopoeia, injectable solutions must meet the criteria given in sections 2.9.19 and 2.9.20.

By “Co-polyamino acid consisting of glutamic or aspartic units” is meant non-cyclic linear chains of glutamic acid or aspartic acid units bound to each other by peptidic bonds, said chains having a C-terminal part, corresponding to the carboxylic acid at one end, and a N-terminal part, corresponding to the amine at the other end of the chain.

By “soluble” is meant as being able to prepare a clear and particle-free solution at a concentration of less than 100 mg/ml in distilled water at 25° C.

The radicals Hy, GpR, GpG, GpH, GpA, GpL and GpC, are each independently identical or different from one residue to another.

In one embodiment, the composition according to the invention is characterized in that Hy comprises from 15 to 100 carbon atoms.

In one embodiment, the composition according to the invention is characterized in that Hy comprises from 30 to 70 carbon atoms.

In one embodiment, the composition according to the invention is characterized in that Hy comprises from 40 to 60 carbon atoms.

In one embodiment, the composition according to the invention is characterized in that Hy comprises from 20 to 30 carbon atoms.

In one embodiment, the composition according to the invention is characterized in that Hy comprises more than 30 carbon atoms.

In the formulas, the * indicates the attachment sites of the hydrophobic radicals to the PLG or between the different GpR, GpG, GpH, GpA, GpL and GpC groups to form amide functions.

The Hy radicals are attached to the PLG via the amide functions.

In one embodiment, r=0 and the hydrophobic radical according to formula X is bound to the PLG via a covalent bond between a carbonyl of the hydrophobic radical and a nitrogen atom borne by the PLG thus forming an amide function resulting from the reaction of an amine function borne by the precursor of the PLG and an acid function borne by the precursor Hy′ of the hydrophobic radical.

In one embodiment, r=1 or 2 and the hydrophobic radical according to formula X is bound to PLG:
via a covalent bond between a nitrogen atom from the hydrophobic radical and a carbonyl borne by the PLG thus forming an amide function resulting from the reaction of an amine function of the precursor -Hy′ of the hydrophobic radical and an acid function borne by the PLG or,
via a covalent bond between a carbonyl from the hydrophobic radical and a nitrogen atom borne by the PLG thus forming an amide function resulting from the reaction of an acid function of the precursor Hy′ of the hydrophobic radical -Hy and an amine function borne by the PLG.

In one embodiment, if GpA is a radical according to Formula VIIIc and r=1, then:
the GpC are directly or indirectly bound to N_{α1} and N_{α2} and the PLG is directly or indirectly bound via GpR to N_{β1}, or
the GpC are directly or indirectly bound to N_{α1} and N_{β1}, and the PLG is directly or indirectly bound via GpR to N_{α2}, or
the GpC are directly or indirectly bound to N_{α2} and N_{β1}, and the PLG is directly or indirectly bound via GpR to N_{α1}.

In one embodiment, if GpA is a radical according to Formula VIIIc and r=0, then:
the GpC are directly or indirectly bound to N_{α1} and N_{α2} and the PLG is directly or indirectly bound to N_{β1}; or
the GpC are directly or indirectly bound to N_{α1} and N_{β1}, and the PLG is directly or indirectly bound to N_{α2}; or
the GpC are directly or indirectly bound to N_{α2} and N_{β1}, and the PLG is directly or indirectly bound to N_{α1}.

In one embodiment, if GpA is a radical according to Formula VIIId and r=1, then:
the GpC are directly or indirectly bound to N_{α1}, N_{α2} and N_{β1} and the PLG is directly or indirectly bound via GpR to N_{β2}; or
the GpC are directly or indirectly bound to N_{α1}, N_{α2} and N_{β2} and the PLG is directly or indirectly bound via GpR to N_{β1}; or
the GpC are directly or indirectly bound to N_{α1}, N_{β1} and N_{β2} and the PLG is directly or indirectly bound via GpR to N_{α2}; or
the GpC are directly or indirectly bound to N_{α2}, N_{β1} and N_{β2} and the PLG is directly or indirectly bound via GpR to N_{α1}.

In one embodiment, if GpA is a radical according to Formula VIIId and r=0, then
the GpC are directly or indirectly bound to N_{α1}, N_{α2} and N_{β1} and the PLG is directly or indirectly bound to N_{β2}; or
the GpC are directly or indirectly bound to N_{α1}, N_{α2} and N_{β2} and the PLG is directly or indirectly bound to N_{β1}; or
the GpC are directly or indirectly bound to N_{α1}, N_{β1} and N_{β2} and the PLG is directly or indirectly bound to N_{α2}; or
the GpC are directly or indirectly bound to N_{α2}, N_{β1} and N_{β2} and the PLG is directly or indirectly bound to N_{α1}.

In one embodiment, when r=2, then the GpR group bound to the PLG is chosen from the GpR according to formula VII.

In one embodiment, when r=2, then the GpR group bound to the PLG is chosen from the GpR according to formula VII and the second GpR is chosen from the GpR according to formula VII″.

In one embodiment, an embodiment, when r=2 then the GpR bound to the PLG is chosen from the GpR according to formula VII″.

In one embodiment, an embodiment, when r=2, then the GpR group bound to the PLG is chosen from the GpR according to formula VII″ and the second GpR is chosen from the GpR according to formula VII.

In one embodiment, a=0,

In one embodiment h=1 and g=0,

In one embodiment h=0 and g=1,

In one embodiment, r=0, g=1 and h=0.

In one embodiment, at least one of the g, h or 1 is different from 0.

In one embodiment, at least one of g and of h is equal to 1.

In one embodiment, at least one of g and h is equal to 1.

In one embodiment a=1 and l=1.

In one embodiment, if l=0, at least one of g and h is equal to 0.

In one embodiment, if l=1, at least one of g and h is equal to 0.

In one embodiment g+h≥2.

In one embodiment g is greater than or equal to 2 (g≥2).

In one embodiment h is greater than or equal to 2 (h≥2).

In one embodiment, g+h≥2 and a and 1 are equal to 0 (a=l=0).

In one embodiment, g+h≥2 and b are equal to 0 (b=0).

In one embodiment g or h is greater than or equal to 2 (g≥2) and b is equal to 0.

In one embodiment, g+h≥2, b is equal to 0 (b=0) and e is equal to 1 (e=1).

In one embodiment g or h is greater than or equal to 2 (g≥2) and b is equal to 0 (b=0) and e is equal to 1 (e=1).

In one embodiment, said at least one hydrophobic radical -Hy is chosen among the radicals according to formula X in which r=2 according to formula Xc′, as defined below:

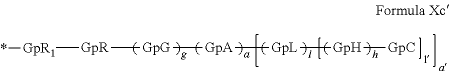

in which GpR₁ is a radical according to formula VII.

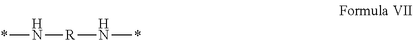

in which GpR, GpG, GpA, GpL, GpH, GpC, R, a, a′, g, h, l and l′ have the definitions given above.

In one embodiment, said at least one hydrophobic radical -Hy is chosen among the radicals according to formula X in which r=2 according to formula Xc′, as defined below:

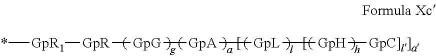

in which GpR₁ is a radical according to formula VII″.

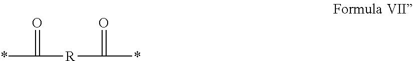

in which GpR, GpG, GpA, GpL, GpH, GpC, R, a, a′, g, h, l and l′ have the definitions given above.

In one embodiment, g=h=0, a=1, GpA is a radical according to formula VIII with s′=1 and A′ according to formula VIII′ or VIII″, and l=1.

In one embodiment, said at least one hydrophobic radical -Hy is chosen among the radicals according to formula X in which r=2 according to formula Xc′, as defined below:

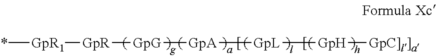

in which GpR₁ is a radical according to formula VII.

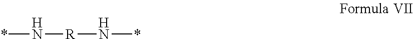

in which GpR, GpG, GpA, GpL, GpH, GpC, R, a, a′, g, h, l and l′ have the definitions given above.

In one embodiment, said at least one hydrophobic radical -Hy is chosen among the radicals according to formula X in which r=2 according to formula Xc′, as defined below:

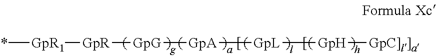

in which GpR₁ is a radical according to formula VII″.

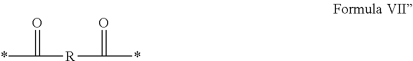

in which GpR, GpG, GpA, GpL, GpH, GpC, R, a, a′, g, h, l and l′ have the definitions given above.

In one embodiment, said at least one hydrophobic radical -Hy is chosen among the radicals according to formula X in which
l=0,
according to formula Xb′ as defined below

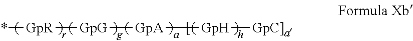

in which
GpR is chosen among the radicals according to formulas VII, VII′ or VII″:

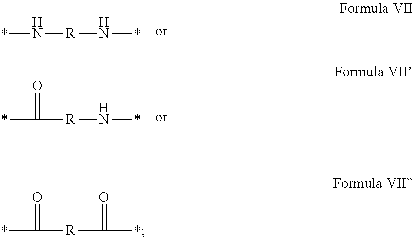

GpG is chosen among the radicals according to formulas XI, or XI′:

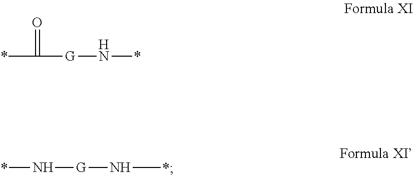

GpA is chosen among the radicals according to formula VIII in which s′=1 represented by Formula VIIIa or according to formula VIII in which s′=0 represented by Formula VIIIb:

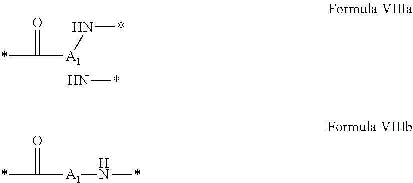

GpC is a radical according to formula IX:

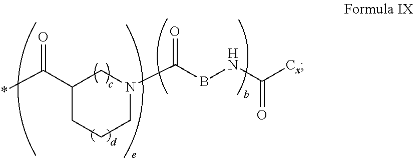

the * indicates the attachment sites of the different groups bound by amide functions;
a is an integer equal to 0 or 1 and a′=1 if a=0 and a′=1 or a′=2 if a=1;
a′ is an integer equal to 1 or 2, and
   if a′ is equal to 1 then a is equal to 0 or to 1 and GpA is a radical according to formula VIIIb and,
   if a′ is equal to 2 then a is equal to 1, and GpA is a radical according to formula VIIIa;
b is an integer equal to 0 or 1;
c is an integer equal to 0 or 1, and if c is equal to 0 then d is equal to 1 or 2;
d is an integer equal to 0, 1 or 2;
e is an integer equal to 0 or 1;
g is an integer equal to 0, 1, 2, 3, 4, 5 or 6;
h is an integer equal to 0, to 1, to 2, to 3 to 4 to 5 or to 6, and at least one of g or h is different from 0;
r is an integer equal to 0, 1 or to 2, and
s′ is an integer equal to 0 or 1;
A₁ is a linear or branched alkyl radical, and optionally substituted by a radical from a saturated, unsaturated or aromatic ring, comprising from 1 to 6 carbon atoms;
B is a linear or branched alkyl radical, optionally comprising an aromatic ring comprising from 1 to 9 carbon atoms or an unsubstituted ether or polyether radical comprising from 4 to 14 carbon atoms and from 1 to 5 oxygen atoms;
Cₓ is a linear or branched monovalent alkyl radical, optionally comprising a cyclic part, in which x indicates the number of carbon atoms and:
   When the hydrophobic radical -Hy bears 1 -GpC, then 9≤x≤25,
   When the hydrophobic radical -Hy bears 2 -GpC, then 9≤x≤15,
   When the hydrophobic radical -Hy bears 3 -GpC, then 7≤x≤13,
   When the hydrophobic radical -Hy bears 4 -GpC, then 7≤x≤11,
   When the hydrophobic radical -Hy bears at least 5 -GpC, then 6≤x≤11,
G is a branched alkyl radical of 1 to 8 carbon atoms, said alkyl radical bearing one or more free carboxylic acid function(s),
R is a radical chosen in the group consisting of a linear or branched divalent alkyl radical comprising from 1 to 12 carbon atoms, a linear or branched divalent alkyl radical comprising from 1 to 12 carbon atoms bearing one or more —CONH₂ functions or an unsubstituted ether or polyether radical comprising from 4 to 14 carbon atoms and from 1 to 5 oxygen atoms:
The hydrophobic radical(s) Hy according to formula X being bound to PLG:
   via a covalent bond between a carbonyl of the hydrophobic radical and a nitrogen atom borne by the PLG thus forming an amide function resulting from the reaction of an amine function borne by the PLG and an acid function borne by the precursor of the hydrophobic radical, and
   via a covalent bond between a nitrogen atom from the hydrophobic radical and a carbonyl borne by the PLG thus forming an amide function resulting from the reaction of an amine function of the precursor -Hy′ of the hydrophobic radical and an acid function borne by the PLG,
the ratio M between the number of hydrophobic radicals and the number of glutamic or aspartic units being between 0<M≤0.5;
when several hydrophobic radicals are borne by a co-polyamino acid then they are identical or different,
the free carboxylic acid functions being in the form of an alkali metal salt chosen in the group consisting of Na⁺ and K⁺.

In one embodiment, said at least one hydrophobic radical -Hy is chosen among the radicals according to formula X as defined below in which l=0,
GpA is chosen among the radicals according to formula VIII in which s′=1 and A′ is chosen among the radicals according to formula VIII″ or VIII′″,
according to formula Xb′ as defined below:

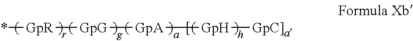

in which
GpR is chosen among the radicals according to formulas VII, VII′ or VII″:

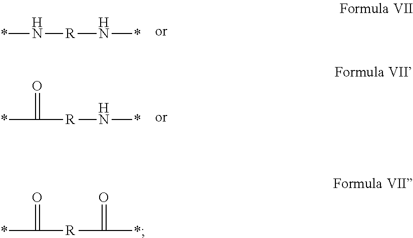

GpG is chosen among the radicals according to formulas XI, or XI′:

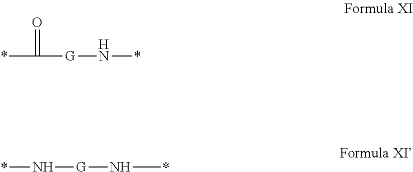

GpA is chosen among the radicals according to formulas VIIIc or VIIId:

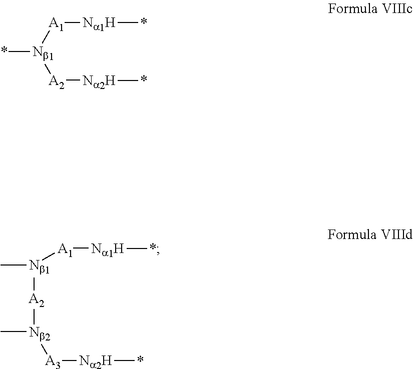

GpC is a radical according to formula IX:

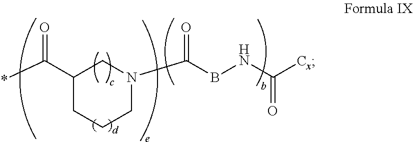

the * indicates the attachment sites of the different groups bound by amide functions;
a is an integer equal to 0 or 1 and a′=1 if a=0 and a′=2 or 3 if a=1;
a′ is an integer equal to 2 or 3, and
   if a′ is equal to 1 then a is equal to 0 and,
   if a′ is equal to 2 or 3 then a is equal to 1, and GpA is a radical according to formula VIIIc or VIIId;
b is an integer equal to 0 or 1;
c is an integer equal to 0 or 1, and if c is equal to 0 then d is equal to 1 or 2;
d is an integer equal to 0, 1 or 2;
e is an integer equal to 0 or 1;
g is an integer equal to 0, 1, 2, 3, 4, 5 or 6;
h is an integer equal to 0, to 1, to 2, to 3 to 4 to 5 or to 6, and at least one of g or h is different from 0;
r is an integer equal to 0, 1 or to 2, and
s′ is an integer equal to 1;
A₁, A₂, A₃, which are identical or different, are linear or branched alkyl radicals, and optionally substituted by a radical from a saturated, unsaturated or aromatic ring, comprising from 1 to 6 carbon atoms;
B is a linear or branched alkyl radical, optionally comprising an aromatic ring comprising from 1 to 9 carbon atoms or an unsubstituted ether or polyether radical comprising from 4 to 14 carbon atoms and from 1 to 5 oxygen atoms;
Cₓ, is a linear or branched monovalent alkyl radical, optionally comprising a cyclic part, in which x indicates the number of carbon atoms and:
   When the hydrophobic radical -Hy bears 1 -GpC, then 9≤x≤25,
   When the hydrophobic radical -Hy bears 2 -GpC, then 9≤x≤15,
   When the hydrophobic radical -Hy bears 3 -GpC, then 7≤x≤13,
   When the hydrophobic radical -Hy bears 4 -GpC, then 7≤x≤11,
   When the hydrophobic radical -Hy bears at least 5 -GpC, then 6≤x≤11,
The hydrophobic radical(s) Hy according to formula X being bound to PLG:
   via a covalent bond between a carbonyl of the hydrophobic radical and a nitrogen atom borne by the PLG thus forming an amide function resulting from the reaction of an amine function borne by the PLG and an acid function borne by the precursor -Hy′ of the hydrophobic radical, and
   via a covalent bond between a nitrogen atom from the hydrophobic radical and a carbonyl borne by the PLG thus forming an amide function resulting from the reaction of an amine function of the precursor -Hy′ of the hydrophobic radical and an acid function borne by the PLG,
G is a branched alkyl radical of 1 to 8 carbon atoms, said alkyl radical bearing one or more free carboxylic acid function(s),
R is a radical chosen in the group consisting of a linear or branched divalent alkyl radical comprising from 1 to 12 carbon atoms, a linear or branched divalent alkyl radical comprising from 1 to 12 carbon atoms bearing one or more —CONH₂ functions or an unsubstituted ether or polyether radical comprising from 4 to 14 carbon atoms and from 1 to 5 oxygen atoms:
the ratio M between the number of hydrophobic radicals and the number of glutamic or aspartic units being between 0<M≤0.5;
when several hydrophobic radicals are borne by a co-polyamino acid then they are identical or different,
the free carboxylic acid functions being in the form of an alkali metal salt chosen in the group consisting of Na⁺ and K⁺.

In one embodiment, said at least one hydrophobic radical -Hy is chosen among the radicals according to formula X in which a=1 and a′=1 according to formula Xa, as defined below:

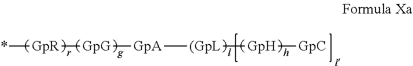

in which GpA is a radical according to formula VIII and A′ is chosen among the radicals according to formula VIII′ with s′=0 and GpA is a radical according to formula VIIIb

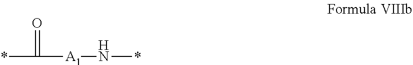

And GpR, GpG, GpL, GpH, GpC, A₁, r, g, h, l and l′ have the definitions given above.

In one embodiment, said at least hydrophobic radical -Hy is chosen among the radicals according to formula X in which a=1 according to formula Xb, as defined below:

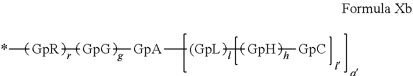

in which GpA is a radical according to formula VIII and A′ is chosen among the radicals according to formula VIII′ with s′=1 and GpA is a radical according to formula VIIIa

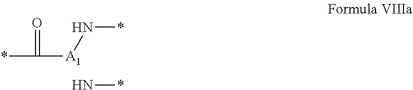

And GpR, GpG, GpL, GpH, GpC, A₁, a′, r, g, h, l and l′ have the definitions given above.

In one embodiment, said at least hydrophobic radical -Hy is chosen among the radicals according to formula X in which a=1 as defined below:

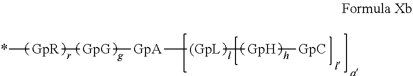

in which GpA is a radical according to formula VIII and A is chosen among the radicals according to formula VIII″ with s′=1 and GpA is a radical according to formula VIIIc

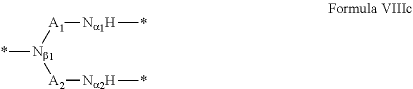

And GpR, GpG, GpL, GpH, GpC, A₁, A₂, r, g, h, a′, l and l′ have the definitions given above.

In one embodiment, said at least hydrophobic radical -Hy is chosen among the radicals according to formula X in which a=1 as defined below:

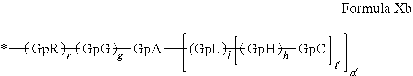

in which GpA is a radical according to formula VIII and A is chosen among the radicals according to formula VIII′″ with s′=1, and GpA is a radical according to formula VIIId

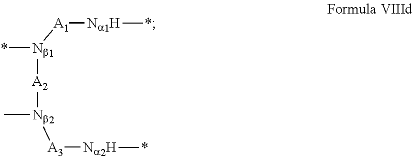

And GpR, GpG, GpL, GpH, GpC, A₁, A₂, A₃, a′, r, g, h, l and l′ have the definitions given above.

In one embodiment, the composition according to the invention is characterized in that said hydrophobic radicals are chosen among the radicals according to formula X in which GpA is a radical according to formula VIIIb, a′=1 and l=0, represented by the following Formula Xe:

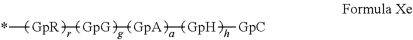

GpR, GpG, GpA, GpH, GpC, r, g, h, and a have the definitions given above.

In one embodiment, r=0, and GpA is chosen among the radicals according to formula VIIIa or VIIIb.

In one embodiment, r=0, g=0 and GpA is chosen among the radicals according to formula VIIIa or VIIIb.

In one embodiment, r=0, and GpA is chosen among the radicals according to formula VIIIa or VIIIb and h=0.

In one embodiment, said at least one hydrophobic radical -Hy is chosen among the radicals according to formula X in which r=1 according to formula Xc, as defined below:

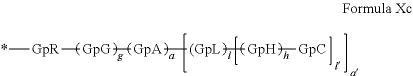

in which GpR is a radical according to formula VII.

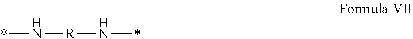

And GpG, GpA, GpL, GpH, GpC, R, a, a′, g, h, l, a′ and l′ have the definitions given above.

In one embodiment, said at least one hydrophobic radical -Hy is chosen among the radicals according to formula X in which r=1 according to formula Xc, as defined below:

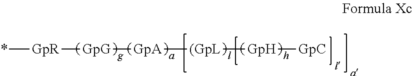

in which GpR is a radical according to formula VII′.

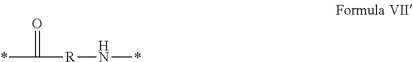

And GpG, GpA, GpL, GpH, GpC, R, a, a′, g, h, l, a′ and l′ have the definitions given above.

In one embodiment, said at least one hydrophobic radical -Hy is chosen among the radicals according to formula X in which r=1 according to formula Xc as defined below:

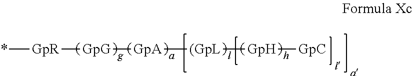

in which GpR is a radical according to formula VII″.

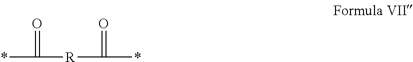

In one embodiment, r=1 and GpR is chosen among the radicals according to formula VII′ or VII″ and h=0.

In one embodiment, r=1, g=0 and GpR is a radical according to formula VII′ and h=0.
In one embodiment, r=1, g=0 and GpR is a radical according to formula VII′ and h=1.

In one embodiment, r=1, g=0, GpR is a radical according to formula VII′, GpA is chosen among the radicals according to formula VIIIa or VIIIb and h=0.

In one embodiment, r=1, g=0, GpR is a radical according to formula VII′, GpA is chosen among the radicals according to formula VIIIa or VIIIb and h=1.

In one embodiment, r=1, g=0, GpR is a radical according to formula VII′, GpA is a radical according to formula VIIIa and h=0.

In one embodiment, r=1, g=0, GpR is a radical according to formula VII′, GpA is a radical according to formula VIIIa and h=1.

In one embodiment, r=1, g=0, GpR is a radical according to formula VII′, GpA is a radical according to formula VIIIb and h=0.

In one embodiment, r=1, g=0, GpR is a radical according to formula VII′, GpA is a radical according to formula VIIIb and h=1.

In one embodiment, said at least one hydrophobic radical -Hy is chosen among the radicals according to formula X as defined below:

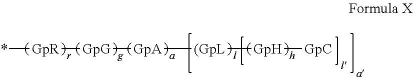

in which GpC is a radical according to formula IX in which e=0, and GpC is a radical according to formula IXa

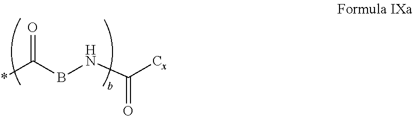

In one embodiment, said at least one hydrophobic radical -Hy is chosen among the radicals according to formula X as defined below:

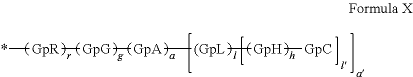

in which GpC is a radical according to formula IX in which e=1, b=0 and GpC is a radical according to formula IXd

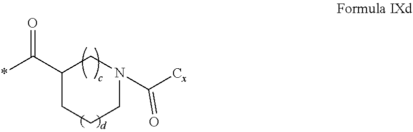

In one embodiment, said at least one hydrophobic radical -Hy is chosen among the radicals according to formula X as defined below:

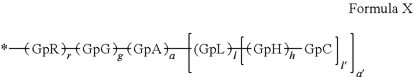

in which GpC is a radical according to formula IX in which e=1, and GpC is a radical according to formula IXb

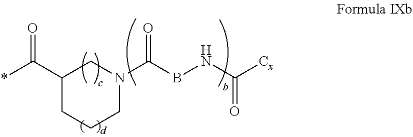

In one embodiment, said at least one hydrophobic radical -Hy is chosen among the radicals according to formula X in which r, g, a, 1, h are equal to 0, according to formula Xd, as defined below:

*-GpC Formula Xd.

in which GpC is a radical according to formula IX in which e=0, b=0 and GpC is a radical according to formula IXc

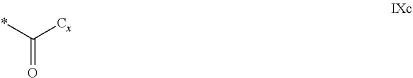

In one embodiment, the composition according to the invention is characterized in that said hydrophobic radicals are chosen among the radicals according to formula X hydrophobic in which a′=2 and a=1 and l=0 represented by the following Formula Xf:

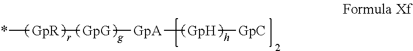

GpR, GpG, GpA, GpH, GpC, r, g and h have the definitions given above.

In one embodiment, the composition according to the invention is characterized in that the said hydrophobic radicals are chosen among the hydrophobic radicals according to formula X in which h=0, l=0 and l′=1, represented by the following Formula Xg:

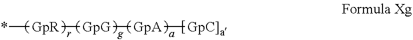

GpR, GpG, GpA, GpC, r, g, a and a′ have the definitions given above.

In one embodiment, the composition according to the invention is characterized in that said hydrophobic radicals are chosen among the hydrophobic radicals according to formula X in which h=0, a′=1, represented by the following Formula Xh:

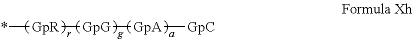

GpR, GpG, GpA, GpC, r, a and g have the definitions given above.

In one embodiment, the composition according to the invention is characterized in that said hydrophobic radicals are chosen among the hydrophobic radicals according to formula X in which h=0, a′=2 and a=1, represented by the following Formula Xi:

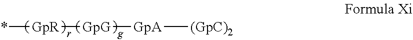

GpR, GpG, GpA, GpC, r and g have the definitions given above.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 2 to 12 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 2 to 6 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 2 to 6 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 2 to 4 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 2 to 4 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising 2 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 1 to 11 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 1 to 6 carbon atoms.

In one embodiment, the composition is characterized in that the hydrophobic radical according to formulas X, Xa, Xb, Xb′, Xc, Xe, Xf, Xg, Xh and Xi is a radical in which R is a divalent alkyl radical comprising 2 to 5 carbon atoms and one or more amide functions (—CONH2).

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising from 2 to 5 carbon atoms and bearing one or more amide functions (—CONH2).

In one embodiment, the composition is characterized in that the hydrophobic radical according to formulas X, Xa, Xb, Xb′, Xc, Xe, Xf, Xg, Xh and Xi is a radical in which R is a radical chosen in the group consisting of the radicals represented by the formulas below:

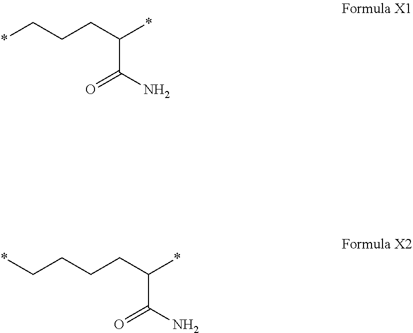 [SMILES: *CCCC(*)C(N)=O ; *CCCCC(*)C(N)=O]

In one embodiment, the composition is characterized in that the hydrophobic radical according to formulas X, Xa, Xb, Xb′, Xc, Xe, Xf, Xg, Xh and Xi is a radical in which R is a radical according to formula X1.

In one embodiment, the composition is characterized in that the hydrophobic radical according to formulas X, Xa, Xb, Xb′, Xc, Xe, Xf, Xg, Xh and Xi is a radical in which R is a radical according to formula X2.

In one embodiment, the composition is characterized in that the hydrophobic radical according to formulas X, Xa, Xb, Xb′, Xc, Xe, Xf, Xg, Xh and Xi is a radical in which R is bound to the co-polyamino acid via an amide function borne by the carbon in the delta or epsilon position (or in position 4 or 5) with respect to the amide function (—CONH₂).

In one embodiment, the composition is characterized in that the hydrophobic radical according to formulas X, Xa, Xb, Xb′, Xc, Xe, Xf, Xg, Xh and Xi is a radical in which R is an unsubstituted linear ether or polyether radical comprising from 4 to 14 carbon atoms and from 1 to 5 oxygen atoms.

In one embodiment, the composition is characterized in that the hydrophobic radical according to formulas X, Xa, Xb, Xb′, Xc, Xe, Xf, Xg, Xh and Xi is a radical in which R is an ether radical.

In one embodiment, the composition is characterized in that the hydrophobic radical according to formulas X, Xa, Xb, Xb′, Xc, Xe, Xf, Xg, Xh and Xi is a radical in which R is a ether radical comprising from 4 to 6 carbon atoms.

In one embodiment, the composition is characterized in that the hydrophobic radical according to formulas X, Xa, Xb, Xb′, Xc, Xe, Xf, Xg, Xh and Xi is a radical in which R is a linear divalent alkyl radical comprising 6 carbon atoms.

In one embodiment, the composition is characterized in that the hydrophobic radical according to formulas X, Xa, Xb, Xb′, Xc, Xe, Xf, Xg, Xh and Xi is a radical in which R is an ether radical represented by the Formula

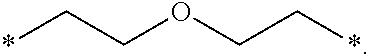

In one embodiment, the composition is characterized in that the hydrophobic radical according to formulas X, Xa, Xb, Xb′, Xc, Xe, Xf, Xg, Xh and Xi is a radical in which R is a polyether radical.

In one embodiment, the composition is characterized in that the hydrophobic radical according to formulas X, Xa, Xb, Xb′, Xc, Xe, Xf, Xg, Xh and Xi is a radical in which R is a linear polyether radical comprising from 6 to 10 carbon atoms and from 2 to 3 oxygen atoms.

In one embodiment, the composition is characterized in that the hydrophobic radical according to formulas X, Xa, Xb, Xb′, Xc, Xe, Xf, Xg, Xh and Xi is a radical in which R is a polyether radical chosen in the group consisting of consisting of the radicals represented by the formulas below:

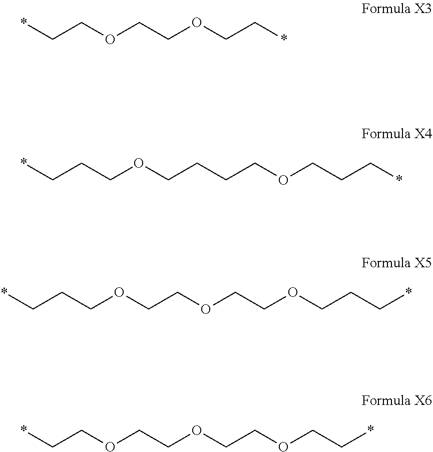 [SMILES: *CCOCCOCC* ; *CCCOCCCCOCCC* ; *CCCOCCOCCOCCC* ; *CCOCCOCCOCC*]

In one embodiment, the composition is characterized in that the hydrophobic radical according to formulas X, Xa, Xb, Xb′, Xc, Xe, Xf, Xg, Xh and Xi is a radical in which R is a radical according to formula X3.

In one embodiment, the composition is characterized in that the hydrophobic radical according to formulas X, Xa, Xb, Xb′, Xc, Xe, Xf, Xg, Xh and Xi is a radical in which R is a radical according to formula X4.

In one embodiment, the composition is characterized in that the hydrophobic radical according to formulas X, Xa, Xb, Xb′, Xc, Xe, Xf, Xg, Xh and Xi is a radical in which R is a polyether radical chosen in the group consisting of the radicals represented by formulas X5 and X6 below:

In one embodiment, the composition is characterized in that the hydrophobic radical according to formulas X, Xa, Xb, Xb′, Xc, Xe, Xf, Xg, Xh and Xi is a radical in which R is a polyether radical according to formula X5.

In one embodiment, the composition is characterized in that the hydrophobic radical according to formulas X, Xa, Xb, Xb′, Xc, Xe, Xf, Xg, Xh and Xi is a radical in which R is a polyether radical according to formula X6.

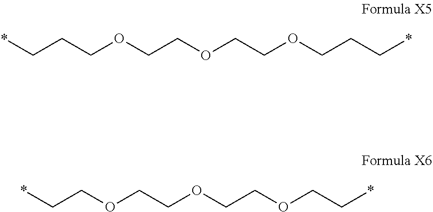 [SMILES: *CCCOCCOCCOCCC* ; *CCOCCOCCOCC*]

In one embodiment, the composition is characterized in that the hydrophobic radical according to formulas X, Xa, Xb, Xb′, Xc, Xe, Xf, Xg, Xh and Xi is a radical in which the radical GpG and/or GpH is according to formula XI′ in which G is an alkyl radical comprising 6 carbon atoms represented by Formula Z below:

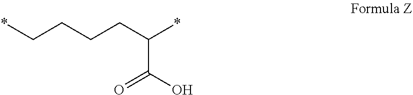 [SMILES: *CCCCC(*)C(=O)O]

In one embodiment, the composition is characterized in that the hydrophobic radical according to formulas X, Xa, Xb, Xb′, Xc, Xe, Xf, Xg, Xh and Xi is a radical in which the radical GpG and/or GpH is according to formula XI in which G is an alkyl radical comprising 4 carbon atoms represented by Formula Z below:

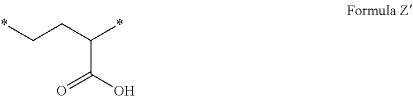 [SMILES: *CCC(*)C(=O)O]

In one embodiment, the composition is characterized in that the hydrophobic radical according to formulas X, Xa, Xb, Xb′, Xc, Xe, Xf, Xg, Xh and Xi is a radical in which the radical GpG and/or GpH is according to formula XI in which G is an alkyl radical comprising 4 carbon atoms represented by —(CH₂)₂—CH(COOH)—.

In one embodiment, the composition is characterized in that the hydrophobic radical according to formulas X, Xa, Xb, Xb′, Xc, Xe, Xf, Xg, Xh and Xi is a radical in which the radical GpG and/or GpH is according to formula XI in which G is an alkyl radical comprising 4 carbon atoms represented by —CH((CH₂)₂COOH)—.

In one embodiment, the composition is characterized in that the hydrophobic radical according to formulas X, Xa, Xb, Xb′, Xc, Xe, Xf, Xg, Xh and Xi is a radical in which the radical GpG and/or GpH is according to formula XI in which G is an alkyl radical comprising 3 carbon atoms represented by —CH₂—CH—(COOH).

In one embodiment, the composition is characterized in that the hydrophobic radical according to formulas X, Xa, Xb, Xb′, Xc, Xe, Xf, Xg, Xh and Xi is a radical in which the radical GpG and/or GpH is according to formula XI in which G is an alkyl radical comprising 3 carbon atoms represented by —CH(CH₂)COOH)—.

In one embodiment, the composition is characterized in that the hydrophobic radical according to formulas X, Xa, Xb, Xb′, Xc, Xe, Xf, Xg, Xh and Xi is a radical in which the radical GpA is according to formula VIII and in which A₁, A₂ or A₃ is chosen in the group consisting of consisting of the radicals represented by the Formulas below:

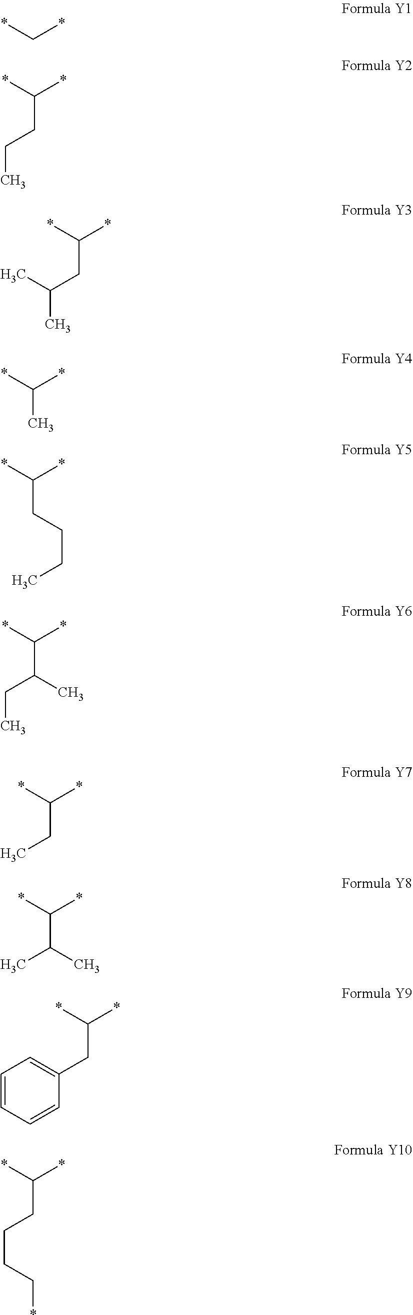

In one embodiment, the composition is characterized in that the hydrophobic radical according to formulas X, Xa, Xb, Xb′, Xc, Xd, Xe, Xf, Xg, Xh and Xi in which the radical GpC according to formula IX is chosen in the group consisting of the radicals according to formulas IXe, IXf or IXg represented below:

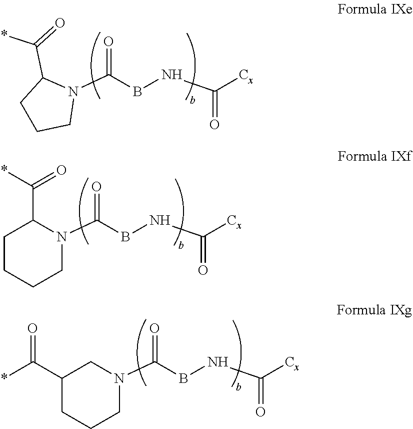

In one embodiment, the composition is characterized in that the hydrophobic radical according to formula X, Xa, Xb, Xb′, Xd, Xc, Xd, Xe, Xf, Xg, Xh and Xi in which the radical GpC according to formula IX is chosen in the group consisting of the radicals according to Formulas IXe, IXf or IXg in which b is equal to 0, which responds respectively to Formulas IXh, IXi, and IXj below:

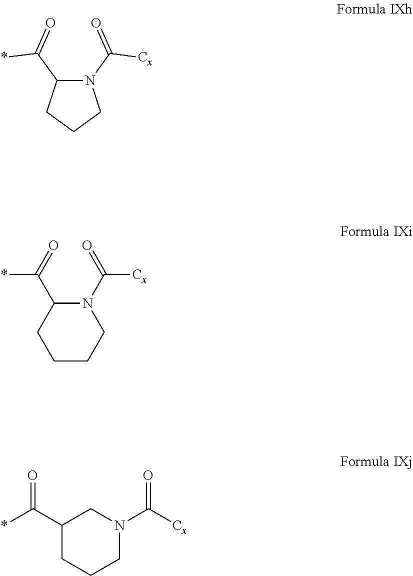

In one embodiment, the composition is characterized in that the hydrophobic radical according to formula X, Xa, Xb, Xb′, Xc, Xd, Xe, Xf, Xg, Xh and Xi is a radical in which the radical GpC responds to Formula IX or IXe in which b=0 and responds to Formula IXh.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of linear alkyl radicals.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of branched alkyl radicals.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of alkyl radicals comprising from 19 to 14 carbon atoms.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of the radicals represented by the formulas below:

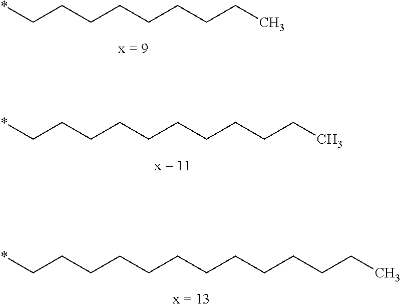

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of alkyl radicals comprising from 15 to 16 carbon atoms.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of the radicals represented by the formulas below:

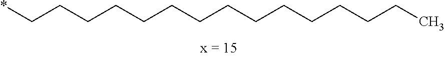 [SMILES: *CCCCCCCCCCCCCCC]

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of the radicals represented by the formulas below:

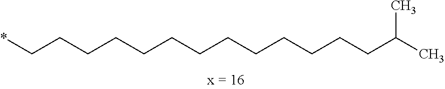 [SMILES: *CCCCCCCCCCCCCC(C)C]

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of alkyl radicals comprising from 17 to 25 carbon atoms.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of alkyl radicals comprising from 17 to 18 carbon atoms.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of the alkyl radicals represented by the formulas below:

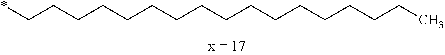 [SMILES: *CCCCCCCCCCCCCCCCC]

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of alkyl radicals comprising from 18 to 25 carbon atoms.

In one embodiment, the composition is characterized in that the hydrophobic radical according to formulas X, Xa, Xb, Xb′, Xc, Xd, Xe, Xf, Xg, Xh and Xi is a radical in which Cx is chosen in the group consisting of the alkyl radicals represented by the formulas below:

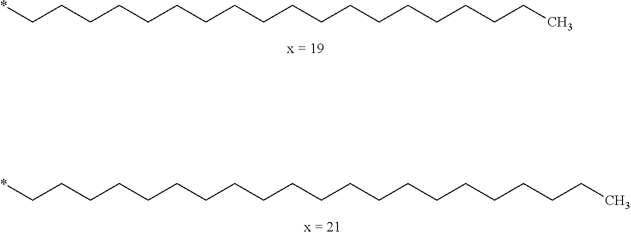 [SMILES: *CCCCCCCCCCCCCCCCCCC ; *CCCCCCCCCCCCCCCCCCCCC]

In one embodiment, the composition is characterized in that the hydrophobic radical is a radical according to formulas X, Xa, Xb, Xb′, Xc, Xd, Xe, Xf, Xg, Xh and Xi in which the Formula IX GpC radical is chosen in the group consisting of radicals in which Cx is chosen in the group consisting of alkyl radicals comprising 14 or 15 carbon atoms.

In one embodiment, the composition is characterized in that the hydrophobic radical is a radical according to formulas X, Xa, Xb, Xb′, Xc, Xd, Xe, Xf, Xg, Xh and Xi in which the radical GpC according to Formula IX is chosen in the group consisting of radicals in which Cx is chosen in the group consisting of the radicals represented by the formulas below:

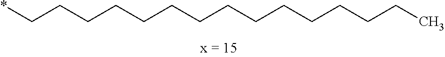 [SMILES: *CCCCCCCCCCCCCCC]

In one embodiment, when a′=1, x is from 11 to 25 (11≤x≤25). In particular, when x is from 15 to 16 (x=15 or 16) then r=1 and R is an ether or polyether radical and when x is greater than 17 (x≥17) then r=1 and R is an ether or polyether radical.

In one embodiment, when a′=2, x is from 9 to 15 (9≤x≤15).

In one embodiment, the hydrophobic radical Hy is chosen in the group of hydrophobic radicals according to formula X, wherein h is greater than or equal to 2 and GpC is according to formula Ixe.

In one embodiment, the hydrophobic radical Hy is chosen in the group of hydrophobic radicals according to formula X, wherein g is greater than or equal to 2 and a, l and h are equal to 0 and GpC is according to formula Ixe.

In one embodiment, the composition is characterized in that the hydrophobic radical is chosen according to formulas X, Xc′, Xa, Xb, Xb′, Xc, Xe, Xg and Xh in which a′=1 and l′=1 and in which Cx is chosen in the group consisting of linear alkyl radicals.

In one embodiment, the composition is characterized in that the hydrophobic radical is chosen according to formulas X, Xc′, Xa, Xb, Xb′, Xc, Xf, Xg and Xi in which a′=2 or l′=2 and in which Cx is chosen in the group consisting of linear alkyl radicals.

In one embodiment, the composition according to the invention is characterized in that the ratio M between the number of hydrophobic radicals and the number of glutamic or aspartic units is from 0.007 to 0.3.

In one embodiment, the composition according to the invention is characterized in that the ratio M between the number of hydrophobic radicals and the number of glutamic or aspartic units is from 0.01 to 0.3.

In one embodiment, the composition according to the invention is characterized in that the ratio M between the number of hydrophobic radicals and the number of glutamic or aspartic units is from 0.02 to 0.2.

In one embodiment, the composition according to the invention is characterized in that the hydrophobic radical responds to Formula X and the ratio M between the number of hydrophobic radicals and the number of glutamic or aspartic units is from 0.007 to 0.15.

In one embodiment, the composition according to the invention is characterized in that the hydrophobic radical responds to Formula X and the ratio M between the number of hydrophobic radicals and the number of glutamic or aspartic units is from 0.01 to 0.1.

In one embodiment, the composition according to the invention is characterized in that the hydrophobic radical responds to Formula X and the ratio M between the number of hydrophobic radicals and the number of glutamic or aspartic units is from 0.02 to 0.08.

In one embodiment, the composition according to the invention is characterized in that the hydrophobic radical responds to Formula X in which radical Cx comprises from 9 to 10 carbon atoms and the ratio M between the number of hydrophobic radicals and the number of glutamic or aspartic units is from 0.03 to 0.15.

In one embodiment, the composition according to the invention is characterized in that the hydrophobic radical responds to Formula X in which radical Cx comprises from 11 to 12 carbon atoms and the ratio M between the number of hydrophobic radicals and the number of glutamic or aspartic units is from 0.015 to 0.1.

In one embodiment, the composition according to the invention is characterized in that the hydrophobic radical responds to Formula X in which radical Cx comprises from 11 to 12 carbon atoms and the ratio M between the number of hydrophobic radicals and the number of glutamic or aspartic units is from 0.02 to 0.08.

In one embodiment, the composition according to the invention is characterized in that the hydrophobic radical responds to Formula X in which radical Cx comprises from 13 to 15 carbon atoms and the ratio M between the number of hydrophobic radicals and the number of glutamic or aspartic units is from 0.01 to 0.1.

In one embodiment, the composition according to the invention is characterized in that the hydrophobic radical responds to Formula X in which radical Cx comprises from 13 to 15 carbon atoms and the ratio M between the number of hydrophobic radicals and the number of glutamic or aspartic units is from 0.01 to 0.06.

In one embodiment, the composition according to the invention is characterized in that the hydrophobic radical responds to Formula X and the ratio M between the number of hydrophobic radicals and the number of glutamic or aspartic units is from 0.007 to 0.3.

In one embodiment, the composition according to the invention is characterized in that the hydrophobic radical responds to Formula X and the ratio M between the number of hydrophobic radicals and the number of glutamic or aspartic units is from 0.01 to 0.3.

In one embodiment, the composition according to the invention is characterized in that the hydrophobic radical responds to Formula X and the ratio M between the number of hydrophobic radicals and the number of glutamic or aspartic units is from 0.015 to 0.2.

In one embodiment, the composition according to the invention is characterized in that the hydrophobic radical responds to Formula X in which radical Cx comprises from 11 to 14 carbon atoms and the ratio M between the number of hydrophobic radicals and the number of glutamic or aspartic units is from 0.1 to 0.2.

In one embodiment, the composition according to the invention is characterized in that the hydrophobic radical responds to Formula X in which radical Cx comprises from 15 to 16 carbon atoms and the ratio M between the number of hydrophobic radicals and the number of glutamic or aspartic units is from 0.04 to 0.15.

In one embodiment, the composition according to the invention is characterized in that the hydrophobic radical responds to Formula X in which radical Cx comprises from 17 to 18 carbon atoms and the ratio M between the number of hydrophobic radicals and the number of glutamic or aspartic units is from 0.02 to 0.06.

In one embodiment, the composition according to the invention is characterized in that the hydrophobic radical responds to Formula X in which radical Cx comprises from 19 to 25 carbon atoms and the ratio M between the number of hydrophobic radicals and the number of glutamic or aspartic units is from 0.01 to 0.06.

In one embodiment, the composition according to the invention is characterized in that the hydrophobic radical responds to Formula X in which radical Cx comprises from 19 to 25 carbon atoms and the ratio M between the number of hydrophobic radicals and the number of glutamic or aspartic units is from 0.01 to 0.05.

In one embodiment, the composition according to the invention is characterized in that the co-polyamino acid bearing carboxylate charges and hydrophobic radicals is chosen among the co-polyamino acids according to formula XXXa′ below:

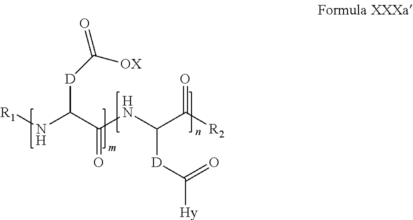

in which,
D is, independently, either a —CH₂— group (aspartic unit) or a —CH₂—CH₂— group (glutamic unit),
Hy is a hydrophobic radical chosen among the hydrophobic radicals according to formulas X, in which r=1 and GpR is a radical according to formula VII,
R₁ is a hydrophobic radical chosen among the hydrophobic radicals according to formula X in which r=0 or r=1 and GpR is a radical according to formula VII′, or a radical chosen in the group consisting of a H, a linear acyl group in C2 to C10, a branched C4 to C10 acyl group, a benzyl, a terminal “amino acid” unit and a pyroglutamate,
R₂ is a hydrophobic radical chosen among the hydrophobic radicals according to formula X in which r=1 and the GpR is a radical according to formula VII or a radical —NR′R″, R′ and R″ which are identical or different being chosen in the group consisting of H, linear or
branched or cyclic C2 to C10 alkyls, the benzyl and said R′ and R″ alkyls which can form together one or more saturated, unsaturated and/or aromatic carbon rings and/or which may contain heteroatoms chosen in the group consisting of O, N and S;
X represents a cationic entity chosen in the group comprising alkaline cations;
n+m represents the degree of polymerization DP of the co-polyamino acid, that is to say the average number of monomeric units per co-polyamino acid chain and 5≤n+m≤250.

When the co-polyamino acid comprises one or more aspartic unit(s), the unit(s) can undergo structural rearrangements.

In one embodiment, the composition according to the invention is characterized in that when the co-polyamino acid comprises aspartate units, then the co-polyamino acid may further comprise monomeric units according to formula XXXI and/or XXXI′:

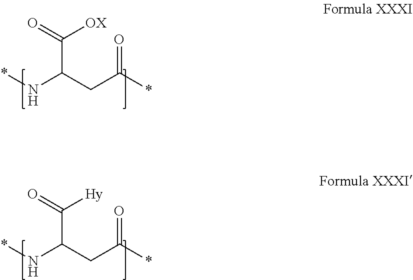

The term “statistical grafting co-polyamino acid” refers to a co-polyamino acid bearing carboxylate charges and at least one hydrophobic radical, a co-polyamino acid according to formula XXXa.

In one embodiment, the composition according to the invention is characterized in that the co-polyamino acid bearing carboxylate charges and hydrophobic radicals is chosen among the co-polyamino acids according to formula XXXa′, in which R₁=R′₁ and R₂=R′₂, according to formula XXXa below:

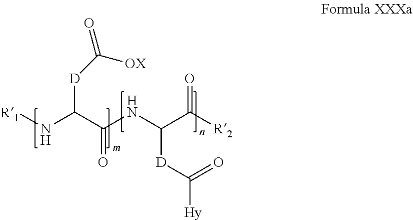

in which,
m, n, X, D and Hy have the definitions given above,
R′₁ is a radical chosen in the group consisting of a H, a linear acyl group in C2 to C10, a branched acyl group in C4 to C10, a benzyl, a terminal “amino acid” unit and a pyroglutamate,
   R′₂ is a radical —NR′R″, R′ and R″ which are identical or different being chosen in the group consisting of H, linear or branched or cyclic C2 to C10 alkyls, the benzyl and said R′ and R″ alkyls which can form together one or more saturated, unsaturated and/or aromatic carbon rings and/or which may contain heteroatoms chosen in the group consisting of O, N and S.

In one embodiment, the composition according to the invention is characterized in that the co-polyamino acid bearing carboxylate charges and hydrophobic radicals is chosen among the co-polyamino acids according to formula XXXa, in which Hy is a radical according to formula X.

In one embodiment, the composition according to the invention is characterized in that the co-polyamino acid bearing carboxylate charges and hydrophobic radicals is chosen among the co-polyamino acids according to formula XXXa, in which Hy is a radical according to formula X, in which r=1.

In one embodiment, the composition according to the invention is characterized in that the co-polyamino acid bearing carboxylate charges and hydrophobic radicals is chosen among the co-polyamino acids according to formula XXXa, in which Hy is a radical according to formula X, in which r=1, and for GpC, b=0.

In one embodiment, the composition according to the invention is characterized in that the co-polyamino acid bearing carboxylate charges and hydrophobic radicals is chosen among the co-polyamino acids according to formula XXXa, in which Hy is according to formula X radical and in which GpC is a radical according to formula IX.

In one embodiment, the composition according to the invention is characterized in that the co-polyamino acid bearing carboxylate charges and hydrophobic radicals is chosen among the co-polyamino acids according to formula XXXa, in which Hy is a radical according to formula X and in which GpC is a radical according to formula IX and r=1.

In one embodiment, the co-polyamino acid is chosen among the co-polyamino acids according to formula XXXb in which the hydrophobic radical -Hy is chosen according to formulas X, Xc′, Xa, Xb′, Xc, Xe, Xg and Xh in which a′=1 and l′=1 and GpC is a radical according to formula IXe.

In one embodiment, the co-polyamino acid is chosen among the co-polyamino acids according to formula XXXb in which the hydrophobic radical -Hy is chosen according to formulas X, Xc′, Xa, Xb′, Xc, Xe, Xg and Xh in which a′=1 and l′=1 and GpC is a radical according to formula IX in which e=0.

In one embodiment, the co-polyamino acid is chosen among the co-polyamino acids according to formula XXXb in which the hydrophobic radical -Hy is chosen according to formulas X, Xc′, Xa, Xb, Xc, Xf, Xg and Xi in which a′=2 and l′=2 and GpC is a radical according to formula IXe.

In one embodiment, the co-polyamino acid is chosen among the co-polyamino acids according to formula XXXb in which the hydrophobic radical -Hy is chosen according to formulas X, Xc′, Xa, Xb, Xc, Xf, Xg and Xi in which a′=2 and l′=2 and GpC is a radical according to formula IX in which e=0.

In one embodiment, the co-polyamino acid is chosen among the co-polyamino acids according to formula XXXa in which the hydrophobic radical -Hy is chosen according to formulas X, Xc′, Xa, Xb′, Xc, Xe, Xg and Xh in which a′=1 and l′=1 and GpC is a radical according to formula IXe.

In one embodiment, the co-polyamino acid is chosen among the co-polyamino acids according to formula XXXa in which the hydrophobic radical -Hy is chosen according to formulas X, Xc′, Xa, Xb, Xc, Xf, Xg and Xi in which a′=2 and l′=2 and GpC is a radical according to formula IXe.

The term “statistical-grafted co-polyamino acid” refers to a co-polyamino acid bearing carboxylate charges and at least one hydrophobic radical, a co-polyamino acid according to formula XXXb.

In one embodiment, the composition according to the invention is characterized in that the co-polyamino acid bearing carboxylate charges and hydrophobic radicals is chosen among the co-polyamino acids according to formula XXXa′ in which n=0 according to formula XXXb below:

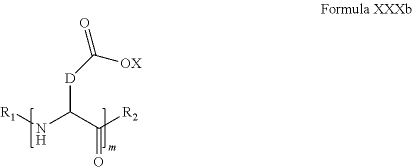

in which m, X, D, R₁ and R₂ have the definitions given above and at least R₁ or R₂ is a hydrophobic radical according to formula X.

In one embodiment, the composition according to the invention is characterized in that the co-polyamino acid bearing carboxylate charges and hydrophobic radicals is chosen among the co-polyamino acids according to formula XXXa′ in which n=0 according to formula XXXb and R₁ or R₂ is a hydrophobic radical according to formula X.

In one embodiment, the composition according to the invention is characterized in that the co-polyamino acid bearing carboxylate charges and hydrophobic radicals is chosen among the co-polyamino acids according to formula XXXb, in which R1=R′1 according to formula XXXb′:

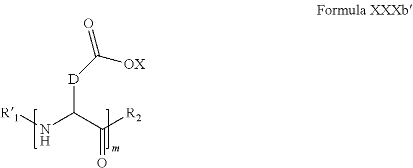

in which m, X, D, R′₁ and R₂ have the definitions given above and R₂ is a hydrophobic radical according to formula X.

In one embodiment, the composition according to the invention is characterized in that the co-polyamino acid bearing carboxylate charges and hydrophobic radicals is chosen among the co-polyamino acids according to formula XXXb, in which R2=R′2 according to formula XXXb″:

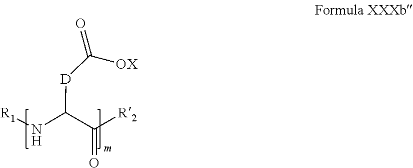

in which m, X, D, R₁ and R′₂ have the definitions given above and R₁ is a hydrophobic radical according to formula X.

In one embodiment, the composition according to the invention is characterized in that the co-polyamino acid bearing carboxylate charges and hydrophobic radicals is chosen among the co-polyamino acids according to formula XXXb or XXXb″ in which R₁ is a hydrophobic radical according to formula X and GpR is according to formula VII′.

In one embodiment, the composition according to the invention is characterized in that the co-polyamino acid bearing carboxylate charges and hydrophobic radicals is chosen among the co-polyamino acids according to formula XXXb or XXXb″ in

which R₁ is a hydrophobic radical according to formula X and GpR is according to formula VII″.

In one embodiment, the composition according to the invention is characterized in that the co-polyamino acid bearing carboxylate charges and hydrophobic radicals is chosen among the co-polyamino acids according to formula XXXb or XXXb″ in which R₁ is a hydrophobic radical according to formula X and GpR is according to formula VII′ and GpC is according to formula IX.

In one embodiment, the composition according to the invention is characterized in that the co-polyamino acid bearing carboxylate charges and hydrophobic radicals is chosen among the co-polyamino acids according to formula XXXb or XXXb″ in which R₁ is a hydrophobic radical according to formula X and GpR is according to formula VII′ and GpC is according to formula IX.

In one embodiment, the composition according to the invention is characterized in that the co-polyamino acid bearing carboxylate charges and hydrophobic radicals is chosen among the co-polyamino acids according to formula XXXb or XXXb′ in which R₂ is a hydrophobic radical according to formula X in which r=1 and GpR is according to formula VII.

In one embodiment, the composition is characterized in that the co-polyamino acid bearing carboxylate charges and hydrophobic radicals is chosen among the co-polyamino acids according to formula XXXa′ in which at least one of R₁ or R₂ is a hydrophobic radical such as defined above according to formula XXX below:

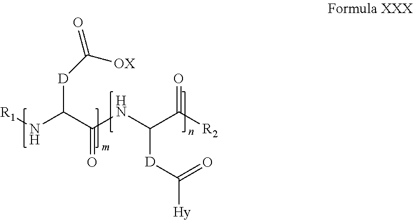

in which,
   D is, independently, either a —CH₂— group (aspartic unit) or a —CH₂—CH₂— group (glutamic unit),
   Hy is a hydrophobic radical chosen among the hydrophobic radicals according to formulas X, in which r=1 and GpR is a radical according to formula VII,
   R₁ is a hydrophobic radical chosen among the hydrophobic radicals according to formula X in which r=0 or r=1 and GpR is a radical according to formula VII′, or a radical chosen in the group consisting of a H, a linear acyl group in C2 to C10, a branched C4 to C10 acyl group, a benzyl, a terminal “amino acid” unit and a pyroglutamate,
   R₂ is a hydrophobic radical chosen among the hydrophobic radicals according to formula X in which r=1 and the GpR is a radical according to formula VII or a radical —NR′R″, R′ and R″ which are identical or different being chosen in the group consisting of H, linear or branched or cyclic C2 to C10 alkyls, the benzyl and said R′ and R″ alkyls which can form together one or more saturated, unsaturated and/or aromatic carbon rings and/or which may contain heteroatoms chosen in the group consisting of O, N and S,
   at least one of R₁ or R₂ is a hydrophobic radical as defined above,
   X represents a H, or a cationic entity chosen in the group comprising metal cations;
   n+m represents the degree of polymerization DP of the co-polyamino acid, that is to say the average number of monomeric units per co-polyamino acid chain and 5≤n+m≤250.

In one embodiment, the composition according to the invention is characterized in that the co-polyamino acid bearing carboxylate charges and hydrophobic radicals is chosen among the co-polyamino acids according to formulas XXX, XXXa, XXXa′, XXXb, XXXb′ or XXXb″ in which group D is a —CH₂— group (aspartic unit).

In one embodiment, the composition according to the invention is characterized in that the co-polyamino acid bearing carboxylate charges and hydrophobic radicals is chosen among the co-polyamino acids according to formulas XXX, XXXa, XXXa′, XXXb, XXXb′ or XXXb″ in which group D is a —CH₂—CH₂— group (glutamic unit).

In one embodiment, the composition according to the invention is characterized in that R₁ is a radical chosen in the group consisting of a linear acyl group in C₂ to C₁₀, a branched acyl group in C₄ to C₁₀, a benzyl, a terminal “amino acid” unit and a pyroglutamate.

In one embodiment, the composition according to the invention is characterized in that R₁ is a radical chosen in the group consisting of a linear acyl group in C₂ to C₁₀ or a branched acyl group in C₄ to C₁₀.

In one embodiment, the composition according to the invention is characterized in that the co-polyamino acid bearing carboxylate charges and hydrophobic radicals is chosen among the co-polyamino acids according to formulas XXXa, XXXb, XXXb′ or XXXb″ in which the co-polyamino acid is chosen among the co-polyamino acids in which group D is a —CH₂— group (aspartic unit).

In one embodiment, the composition according to the invention is characterized in that the co-polyamino acid bearing carboxylate charges and hydrophobic radicals is chosen among the co-polyamino acids according to formulas XXXa, XXXb, XXXb′ or XXXb″ in which the co-polyamino acid is chosen among the co-polyamino acids in which group D is a —CH₂—CH₂— group (glutamic unit).

In one embodiment, the composition according to the invention is characterized in that the co-polyamino acid bearing carboxylate charges and at least one hydrophobic radical -Hy is chosen among the co-polyamino acids according to formula XXXa below:

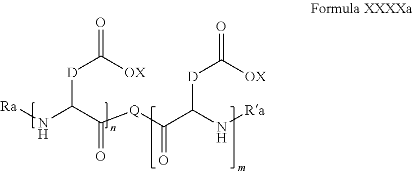

in which,
D is, independently, either a —CH₂— group (aspartic unit) or a —CH₂—CH₂— group (glutamic unit),
X represents a cationic entity chosen in the group comprising the alkaline cations, -,
Ra and R′ a, which are identical or different, are either a hydrophobic radical -Hy or a radical chosen in the group consisting of a H, a linear acyl group in C2 to C10, a branched acyl group in C3 to C10, a benzyl, a terminal “amino acid” unit and a pyroglutamate,
at least one of Ra and R′ a being a hydrophobic radical -Hy,
Q has the meaning given above
-Hy has the meanings given below.
n+m represents the degree of polymerization DP of the co-polyamino acid, that is to say the average number of monomeric units per co-polyamino acid chain and 5≤n+m≤250.

In one embodiment, the composition according to the invention is characterized in that the co-polyamino acid bearing carboxylate charges and at least one hydrophobic radical -Hy is chosen among the co-polyamino acids according to formula XXXa in which Rₐ and R′ₐ, identical, are a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXa in which Rₐ and R′ₐ, different, are a hydrophobic radicals -Hy.

In one embodiment, the composition according to the invention is characterized in that the co-polyamino acid bearing carboxylate charges and at least one hydrophobic radical -Hy is chosen among the co-polyamino acids according to formula XXXXa in which Rₐ is a hydrophobic radical -Hy and R′ₐ is not a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXa in which R′ₐ is a hydrophobic radical -Hy and Rₐ is not a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXa′ below:

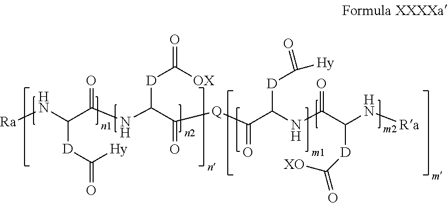

### Wherein:

D, X, Ra and R′a have the definitions given above,
Q and Hy have the meanings given above,
n₁+m₁ represents the number of glutamic units or aspartic units of the PLG chains of the co-polyamino acid bearing a radical -Hy,
n₂+m₂ represents the number of glutamic units or aspartic units of the PLG chains of the co-polyamino acid bearing a radical -Hy,
n₁+n₁=n′ and m₁+m₂=m′

n′+m′ represents the degree of polymerization DP of the co-polyamino acid, that is to say the average number of monomeric units per co-polyamino acid chain and 5≤n′+m′≤250.

In one embodiment, the composition according to the invention is characterized in that the co-polyamino acid bearing carboxylate charges and at least one hydrophobic radical -Hy is chosen among the co-polyamino acids according to formula XXXXa′ in which Ra and R′a, identical, are a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXa′ in which Ra and R′a, different, are a the hydrophobic radicals -Hy.

In one embodiment, the composition according to the invention is characterized in that the co-polyamino acid bearing carboxylate charges and at least one hydrophobic radical -Hy is chosen among the co-polyamino acids according to formula XXXXa′ in which Ra is a hydrophobic radical -Hy and R′a is not a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXa′ in which R′a is a hydrophobic radical -Hy and Ra is not a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXb below:

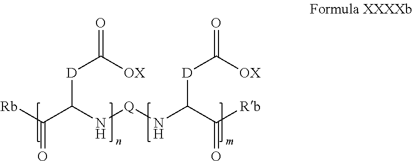

in which,
D and X have the definitions given above,
Rb and R′b, which may be identical or different, are either a hydrophobic radical -Hy or a radical chosen in the group consisting of —OH, an amine group, a terminal “amino acid” unit and a pyroglutamate,
at least one of Rb and R′b is a hydrophobic radical -Hy,
Q and Hy have the meanings given above.
n+m have the same definitions given above.

In one embodiment, the composition according to the invention is characterized in that the co-polyamino acid bearing carboxylate charges and at least one hydrophobic radical -Hy is chosen among the co-polyamino acids according to formula XXXXb in which Rb and R′b, identical, are a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXb in which Rb and R′b, different, are hydrophobic radicals -Hy.

In one embodiment, the composition according to the invention is characterized in that the co-polyamino acid bearing carboxylate charges and at least one hydrophobic radical -Hy is chosen among the co-polyamino acids according to formula XXXXb in which Rb is a hydrophobic radical -Hy and R′b is not a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXb in which R′b is a hydrophobic radical -Hy and Rb is not a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXb′ below:

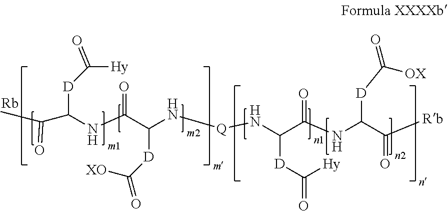

wherein:
D and X have the definitions given above,
Q and Hy have the meanings given above.
Rb and Rb′, which may be identical or different, are either a hydrophobic radical -Hy or a radical chosen in the group consisting of —OH, an amine group, a terminal “amino acid” unit and a pyroglutamate,
at least one of Rb and R′b is a hydrophobic radical -Hy,
n1+m1 represents the number of glutamic units or aspartic units identical,
n2+m2 represents the number of glutamic units or aspartic units of the PLG chains of the co-polyamino acid bearing a radical -Hy,
n1+n2=n′ and m1+m2=m′
n′+m′ represents the degree of polymerization DP of the co-polyamino acid, that is to say the average number of monomeric units per co-polyamino acid chain and 5≤n′+m′≤250.

In one embodiment, the composition according to the invention is characterized in that the co-polyamino acid bearing carboxylate charges and at least one hydrophobic radical -Hy is chosen among the co-polyamino acids according to formula XXXXb′ in which Rb and R′b, identical, are a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXb′ in which Rb and R′b, different, are a the hydrophobic radicals -Hy.

In one embodiment, the composition according to the invention is characterized in that the co-polyamino acid bearing carboxylate charges and at least one hydrophobic radical -Hy is chosen among the co-polyamino acids according to formula XXXXb′ in which Rb is a hydrophobic radical -Hy and R′b is not a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXb′ in which R′b is a hydrophobic radical -Hy and Rb is not a hydrophobic radical -Hy.

In one embodiment, the composition are characterized in that the co-polyamino acid bearing carboxylate charges and hydrophobic radicals is chosen among the co-polyamino acids according to formulas XXXXa, XXXXb, XXXXa′ or XXXXb′ in which group D is a —CH₂—CH₂— group (glutamic unit).

In one embodiment, the composition is characterized in that the co-polyamino acid bearing carboxylate charges and hydrophobic radicals are chosen among the co-polyamino acids according to formulas XXXXa, XXXXa′, XXXXb′ in which group D is a —CH₂— group (aspartic unit).

When the co-polyamino acid comprises one or more aspartic unit(s), the unit(s) can undergo structural rearrangements.

In one embodiment, the composition according to the invention is characterized in that when the co-polyamino acid comprises aspartate units, then the co-polyamino acid may further comprise monomeric units according to formula XXXX and/or XXXX′:

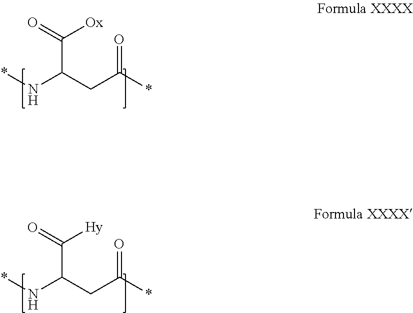

The term “statistical grafting co-polyamino acid” refers to a co-polyamino acid bearing carboxylate charges and at least one hydrophobic radical, represented by a co-polyamino acid according to formula XXXXa′ and XXXXb′.

The term “defined grafting co-polyamino acid” refers to a co-polyamino acid bearing carboxylate charges and at least one hydrophobic radical, represented by a co-polyamino acid according to formula XXXXa and XXXXb.

In one embodiment, the concentration of co-polyamino acids bearing carboxylate charges and hydrophobic radicals is at most 60 mg/ml.

In one embodiment, the concentration of co-polyamino acids bearing carboxylate charges and hydrophobic radicals is at most 40 mg/ml.

In one embodiment, the concentration of co-polyamino acids bearing carboxylate charges and hydrophobic radicals is at most 20 mg/ml.

In one embodiment, the concentration of co-polyamino acids bearing carboxylate charges and hydrophobic radicals is at most 10 mg/ml.

In one embodiment, the concentration of co-polyamino acids bearing carboxylate charges and hydrophobic radicals is at most 5 mg/ml.

In one embodiment, the concentration of co-polyamino acids bearing carboxylate charges and hydrophobic radicals is at most 2.5 mg/ml.

When the co-polyamino acid is chosen among the co-polyamino acids according to formulas XXXXa, XXXXb, XXXXa′ or XXXXb′, it may be represented by a co-polyamino acid according to Formula I:

[Q(PLG)ₖ][Hy]ⱼ[Hy]_{j′} Formula XXXI

### Wherein:

j≥1; 0≤j′≤n′1 and j+j′≥1 and k≥2
said co-polyamino acid according to formula I bearing at least one hydrophobic radical -Hy and carboxylate charges and consisting of at least two chains of glutamic or aspartic units PLG bound together by a linear or branched radical or a Q[-*]ₖ spacer of at least one divalent chain consisting of an alkyl chain comprising one or more heteroatoms chosen in the group consisting of nitrogen and oxygen atoms and/or bearing one or more heteroatoms consisting of nitrogen and oxygen atoms and/or radicals bearing one or more heteroatoms consisting of nitrogen and oxygen atoms and/or carboxyl functions,
   said Q[-*]ₖ radical or spacer being bound to the at least two chains of glutamic or aspartic units PLG by an amide function and,
   said amide functions bonding said Q[-*]ₖ radical or spacer bound to at least two chains of glutamic or aspartic units resulting from the reaction between an amine function and an acid function respectively borne either by the precursor Q′ of the Q[-*]ₖ radical or spacer or by a glutamic or aspartic unit,
   said hydrophobic radical -Hy being bound either to a terminal “amino acid” unit and then j≥1, or to a carboxyl function borne by one of the chains of the glutamic or aspartic unit PLG and then j′=n′1 and n′1 is the average number of monomeric units bearing a hydrophobic radical -Hy.

In one embodiment, the composition according to the invention is characterized in that the co-polyamino acid bearing carboxylate charges and at least one hydrophobic radical -Hy is chosen among the co-polyamino acids according to formula XXXXXa below:

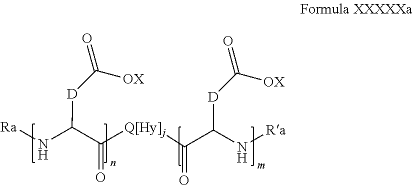

in which,
D is, independently, either a —CH₂— group (aspartic unit) or a —CH₂—CH₂— group (glutamic unit),
X represents a cationic entity chosen in the group comprising the alkaline cations,
Rₐ and Rₐ′, which are identical or different, are a radical chosen in the group consisting of a H, a linear acyl group in C2 to C10, a branched acyl group in C3 to C10, a benzyl, a terminal “amino acid” unit and a pyroglutamate,
Q, Hy and j have the meanings given above.
n+m represents the degree of polymerization DP of the co-polyamino acid, that is to say the average number of monomeric units per co-polyamino acid chain and 5≤n+m≤250;

In one embodiment, the composition according to the invention is characterized in that the co-polyamino acid bearing carboxylate charges and at least one hydrophobic radical -Hy is chosen among the co-polyamino acids according to formula XXXXXa in which Rₐ and Rₐ′, which may be identical or different, are chosen in the group consisting of a H and a pyroglutamate.

In one embodiment, the composition according to the invention is characterized in that the co-polyamino acid bearing carboxylate charges and at least one hydrophobic radical -Hy is chosen among the co-polyamino acids according to formula XXXXXa′ below:

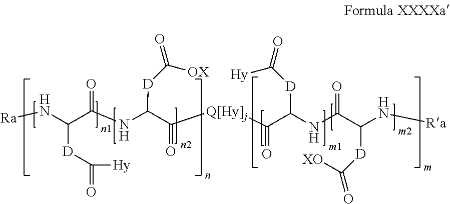

### Wherein:

D is, independently, either a —CH₂— group (aspartic unit) or a —CH₂—CH₂— group (glutamic unit),
X represents a cationic entity chosen in the group comprising the alkaline cations,
Q, Hy and j have the meanings given above.
Rₐ and Rₐ′, which are identical or different, are a radical chosen in the group consisting of a H, a linear acyl group in C2 to C10, a branched acyl group in C3 to C10, a benzyl, a terminal “amino acid” unit and a pyroglutamate,
n₁+m₁ represents the number of glutamic units or aspartic units of the PLG chains of the co-polyamino acid not bearing a radical -Hy,
n₂+m₂ represents the number of glutamic units or aspartic units of the PLG chains of the co-polyamino acid not bearing a radical -Hy,
n₁+n₂=n and m₁+m₂=m
n+m represents the degree of polymerization DP of the co-polyamino acid, that is to say the average number of monomeric units per co-polyamino acid chain and 5≤n+m≤250;

In one embodiment, the composition according to the invention is characterized in that the co-polyamino acid bearing carboxylate charges and at least one hydrophobic radical -Hy is chosen among the co-polyamino acids according to formula XXXXXa″ below:

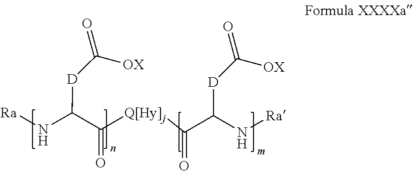

### Wherein:

D is, independently, either a —CH₂— group (aspartic unit) or a —CH₂—CH₂— group (glutamic unit),
X represents a cationic entity chosen in the group comprising the alkaline cations,
Q, Hy and j have the meanings given above.
Rₐ and Rₐ′, which are identical or different, are at least one hydrophobic radical -Hy or a radical chosen in the group consisting of -Hy, a H, a linear acyl group in C2 to C10, a branched acyl group in C3 to C10, a benzyl, a terminal “amino acid” unit and a pyroglutamate,
n+m represents the degree of polymerization DP of the co-polyamino acid, that is to say the average number of monomeric units per co-polyamino acid chain and 5≤n+m≤250;

In one embodiment, the composition according to the invention is characterized in that the co-polyamino acid bearing carboxylate charges and at least one hydrophobic radical -Hy is chosen among the co-polyamino acids according to formula XXXXXb below:

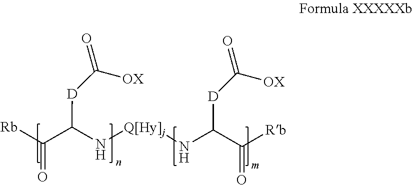

in which,
D is, independently, either a —CH₂— group (aspartic unit) or a —CH₂—CH₂— group (glutamic unit),
R₂ represents a radical or spacer according to formula Q[-*]ᵢ as defined above,
X represents a cationic entity chosen in the group comprising the alkaline cations,
R_{b} et R_{b}′ are a radical —NR′R″, R′ and R″ which are identical or different being chosen in the group consisting of H, linear or branched or cyclic C2 to C10 alkyls, benzyl and said R′ and R″ alkyls which can form together one or more saturated, unsaturated and/or aromatic carbon rings and/or which may contain heteroatoms chosen in the group consisting of O, N and S.
Q, Hy and j have the meanings given above.
n+m represents the degree of polymerization DP of the co-polyamino acid, that is to say the average number of monomeric units per co-polyamino acid chain and 5≤n+m≤250.

In one embodiment, the composition according to the invention is characterized in that the co-polyamino acid bearing carboxylate charges and at least one hydrophobic radical -Hy is chosen among the co-polyamino acids according to formula XXXXXb′ below:

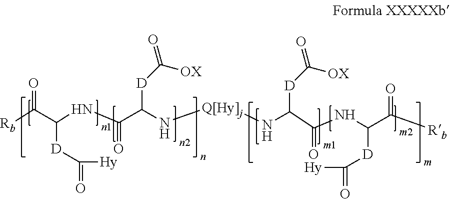

wherein:
D is, independently, either a —CH₂— group (aspartic unit) or a —CH₂—CH₂— group (glutamic unit),
X represents a cationic entity chosen in the group comprising the alkaline cations,
Q, Hy and j have the meanings given above.
R_{b} et R_{b}′ are a radical —NR′R″, R′ and R″ which are identical or different being chosen in the group consisting of H, linear or branched or cyclic C2 to C10 alkyls, benzyl and said R′ and R″ alkyls which can form together one or more saturated, unsaturated and/or aromatic carbon rings and/or which may contain heteroatoms chosen in the group consisting of O, N and S.
n1+m1 represents the number of glutamic units or aspartic units of the PLG chains of the co-polyamino acid not bearing a radical —H,
n2+m2 represents the number of glutamic units or aspartic units of the PLG chains of the co-polyamino acid not bearing a radical —H,
n1+n2=n and m1+m2=m
   n+m represents the degree of polymerization DP of the co-polyamino acid, that is to say the average number of monomeric units per co-polyamino acid chain and 5≤n+m≤250;

In one embodiment, the composition according to the invention is characterized in that the co-polyamino acid bearing carboxylate charges and at least one hydrophobic radical -Hy is chosen among the co-polyamino acids according to formula XXXXXb″ below:

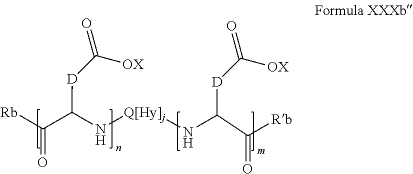

### Wherein:

D is, independently, either a —CH₂— group (aspartic unit) or a —CH₂—CH₂— group (glutamic unit),
X represents a cationic entity chosen in the group comprising the alkaline cations,
R_{b} et R_{b}′, which are identical or different, are at least one hydrophobic radical -Hy and a radical chosen in the group consisting of a hydrophobic radical -Hy and a radical —NR′R″, R′ and R″, which are identical or different being chosen in the group consisting of H, linear or branched or cyclic C2 to C10 alkyls, benzyl and said R′ and R″ alkyls which can form together one or more saturated, unsaturated and/or aromatic carbon rings and/or which may contain heteroatoms chosen in the group consisting of O, N and S;
Q, Hy and j have the meanings given above.
n+m represents the degree of polymerization DP of the co-polyamino acid, that is to say the average number of monomeric units per co-polyamino acid chain and 5≤n+m≤250;

In one embodiment, the composition according to the invention is characterized in that when the co-polyamino acid comprises aspartate units, then the co-polyamino acid may further comprise monomeric units according to formula XXXXX and/or XXXXX′:

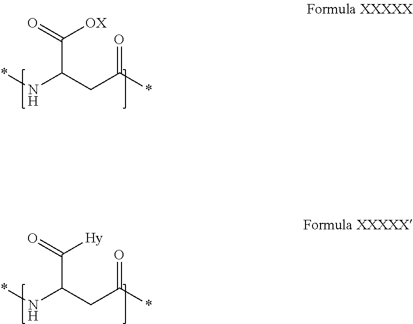

The term “statistical grafting co-polyamino acid” refers to a co-polyamino acid bearing carboxylate charges and at least one hydrophobic radical, a co-polyamino acid according to formula XXXXXa′ and XXXXXb′.

The term “defined grafting co-polyamino acid” refers to a co-polyamino acid bearing carboxylate charges and at least one hydrophobic radical, a co-polyamino acid according to formulas XXXXXa, XXXXXa″′, XXXXXb and XXXXXb″′.

In one embodiment, the composition according to the invention is characterized in that the co-polyamino acid bearing carboxylate charges and hydrophobic radicals is chosen among the co-polyamino acids according to formulas XXXa, XXXXXa′, XXXXXa″, XXXXXb, XXXXXb′ or XXXXXb″ in which the co-polyamino acid is chosen among the co-polyamino acids in which the group D is a —CH₂— group (aspartic unit).

In one embodiment, the composition according to the invention is characterized in that the co-polyamino acid bearing carboxylate charges and hydrophobic radicals is chosen among the co-polyamino acids according to formulas XXXXa, XXXXXa′, XXXXXa″, XXXXXb, XXXXXb′ or XXXXXb″ in which the co-polyamino acid is chosen among the co-polyamino acids in which the group D is a —CH₂—CH₂— group (glutamic unit).

When the co-polyamino acids are co-polyamino acids according to formulas XXXXa, XXXXXa′, XXXXXa″, XXXXXb, XXXXXb′ or XXXXX it may be represented by a co-polyamino acid according to Formula XXXXI′:

Q[Hy]_{j]}[PLG]ₖ[Hy]_{hy}[HY]_{hy′} Formula XXXXXI′

### Wherein:

j≥1; k≥2
hy≥0 and hy′≥0 said co-polyamino acid according to Formula I′ bearing carboxylate charges and consisting of at least two chains of PLG glutamic or aspartic units bound together by a linear or branched Q[-*]ᵢ (i≥3 avec i=j+k) radical or spacer at least trivalent alkyl chain comprising one or more heteroatoms chosen in the group consisting of nitrogen and oxygen atoms and/or bearing one or more heteroatoms consisting of nitrogen and oxygen atoms and/or radicals bearing one or more heteroatoms consisting of nitrogen and oxygen atoms and/or carboxyl functions said radical Q[-*]ᵢ bearing at least one monovalent hydrophobic radical -Hy;
said Q[-*]ᵢ radical or spacer being bound to the at least two chains of PLG glutamic or aspartic units by an amide function and,
said radical or spacer Q[-*]ᵢ being bound to at least one hydrophobic radical Hy according to formula X defined hereafter by an amide function.
said amide functions binding said Q[-*]ᵢ radical or spacer to at least two chains of glutamic or aspartic units resulting from the reaction between an amine function and an acid function respectively borne either by the precursor Q′ of the Q[-*]ᵢ radical or spacer or by a glutamic or aspartic unit.
the amide function binding said Q[-*]ᵢ radical or spacer to at least one hydrophobic radical according to formula X -Hy resulting from the reaction between an amine function and an acid function borne either by the precursor Q′ of the Q[-*]ᵢ radical or spacer or by the precursor Hy′ of the hydrophobic radical -Hy; and
when by and hy′≠0, then at least one hydrophobic radical -Hy is bound either to a terminal “amine acid”, or to a carboxyl function borne by one of the chains from the PLG glutamic or aspartic units.

The Q[-*]ᵢ (i≥3) or Q[-*]ₖ radicals or spacers may be represented by a radical according to Formula QII radical:

Q[-*]ᵢ=([Q′]_{q})[-*]ᵢ Formula QII

Wherein 1≤q≤5

The radicals Q′ being identical or different and chosen in the group consisting of the radicals according to following Formulas QIII to QVI, to form Q[-*]ᵢ (i≥3):

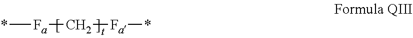

Wherein 1≤t≤8

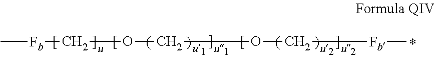

### Wherein:

At least one of the u₁″ or u₂″ is different from 0.
If u₁″≠0 then u₁′≠0 and of u₂″≠0 then u₂′≠0,
u₁′ and u₂′ are identical or different and,
2≤u≤4,
0≤u₁′≤4,
0≤u₁′≤4,
0≤u₂′≤4
0≤u₂″≤4 and,

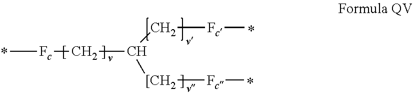

### Wherein:

v, v′ and v″ which may be identical or different,
v+v′+v″≤15,

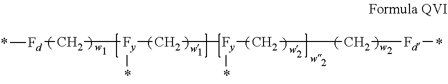

### Wherein:

w₁′ is different from 0,
0≤w₂″≤1,
w₁≤6 and w₁′≤6 and/or w₂≤6 and w₂′≤6
with Fx=Fa, Fb, Fc, Fd, Fa′, Fb′, Fc′, Fc″ and Fd′ representing —NH— or —CO— and Fy functions representing a trivalent nitrogen atom —N═,
two Q′ radicals being bound together by a covalent bond between a carbonyl function, Fx=—CO—, and an amine function Fx=—NH— or Fy=—N═, thus forming an amide bond,

In one embodiment, if Fa and Fa′ are —NH—, then t≥2.

In one embodiment, if Fa and Fa′ are —CO—, then t≥1.

In one embodiment, if Fa and Fa′ are —CO— and —NH—, then t≥1.

In one embodiment, if Fb and Fb′ are —NH—, then u and u₁′≥2 and/or u₂′≥2.

In one embodiment, if Fc, Fc′ and Fc″ are —NH— then at least two of v, v′ and v″ are different than 0.

In one embodiment, if Fc, Fc′ and Fc″ are 2 —NH— and 1 —CO— then at least one of the indices of the —(CH2)- bearing a nitrogen is different than 0.

In one embodiment, if Fc, Fc′ and Fc″ are 1 —NH— and 2 —CO— then there are no conditions.

In one embodiment, if Fc, Fc′ and Fc″ are —CO— then at least one of v, v′ and v″ are different than 0.

In one embodiment, if Fd and Fd′ are —NH—, w1 and w1′≥2 and/or w2 and w′2≥2.

In one embodiment, if Fd and Fd′ are —CO—, w1 and w1′≥1 and/or w2 and w2′≥1.

In one embodiment, if Fd and Fd′ are —CO—, and —NH—, w1 and w1′≥1 and/or w2 and w2′≥1.

Hy and PLG being bound to Q[-*] by an Fx or Fy function by a covalent bond to form an amide bond with a —NH— or —CO— function of the PLG or Hy.

In one embodiment, 1≤q≤4.

In one embodiment, v+v′+v″≤15.

In one embodiment, at least one of the Q′ is a radical according to formula QIII,

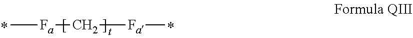

whose precursor is a diamine.

In one embodiment, the precursor of the radical according to Formula QIII is a diamine chosen in the group consisting of ethylene diamine, butylene diamine, hexylene diamine, 1,3-diaminopropane and 1,5-diaminopentane.

In one embodiment, t=2 and the precursor of the radical according to formula QIII is ethylene diamine.

In one embodiment, t=4 and the precursor of the radical according to formula QIII is butylene diamine.

In one embodiment, t=6 and the precursor of the radical according to formula QIII is hexylene diamine.

In one embodiment, t=3 and the precursor of the radical according to formula QIII is 1,3-diaminopropane.

In one embodiment, t=5 and the precursor of the radical according to formula QIII is 1.5-diaminopentane.

In one embodiment, the precursor of the radical according to formula QIII is an amino acid.

In one embodiment, the precursor of the radical according to formula QIII is an amino acid chosen in the group consisting of amino butanoic acid, amino hexanoic acid and beta-alanine.

In one embodiment, t=2 and the precursor of the radical according to formula QIII is beta-alanine.

In one embodiment, t=6 and the precursor of the radical according to formula QIII is an amino hexanoic acid.

In one embodiment, t=4 and the precursor of the radical according to formula QIII is an amino butanoic acid.

In one embodiment, the precursor of the radical according to formula QIII is a diacid.

In one embodiment, the precursor of the radical according to formula QIII is an amino acid chosen in the group consisting of succinic acid, glutaric acid and adipic acid.

In one embodiment, t=2 and the precursor of the radical according to formula QIII is succinic acid.

In one embodiment, t=3 and the precursor of the radical according to formula QIII is glutaric acid.

In one embodiment, t=4 and the precursor of the radical according to formula QIII is adipic acid.

In one embodiment, at least one of the Q′ is a radical according to formula QIV,

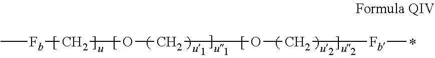

whose precursor is a diamine.

In one embodiment, the precursor of the radical according to formula QIV is a diamine chosen in the group consisting of diethylene glycol diamine, triethylene glycol diamine, 1-amino-4,9-dioxa-12-dodecanamine and 1-amino-4,7,10-trioxa-13-tridecanamine.

In one embodiment, u=u′₁=2, u″₁=1, u″₂=0 and the precursor of the radical according to formula QIV is diethylene glycol diamine.

In one embodiment, u=u′₁=u′₂=2, u″₁=u″₂=1 and the precursor of the radical according to formula QIV is triethylene glycol diamine.

In one embodiment, u=u′₂=3, u′₁=4, u″₁=u″₂=1 and the precursor of the radical according to formula QIV is 4,9-dioxa-1,12-dodecane diamine.

In one embodiment, u=u′₂=3, u′₁=u″₁=2, u″₂=1 and the precursor of the radical according to formula QIV is 4,7,10-trioxa-1,13-tridecane diamine.

In one embodiment, at least one of the Q′ is a radical according to formula QV,

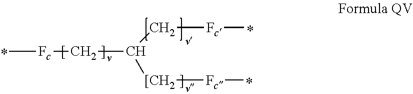

whose precursor is chosen in the group consisting of amino acids.

In one embodiment, the precursor of the radical according to formula QV is an amino acid chosen in the group consisting of lysine, ornithine, and 2,3-diaminopropionic acid.

In one embodiment, at least one of the Q′ is a radical according to formula QV,

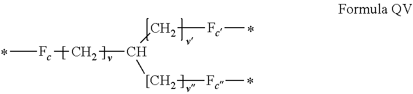

whose precursor is chosen in the group consisting of triacids.

In one embodiment, the precursor of the radical according to formula QV is a triacid chosen in the group consisting of tricarballylic acid.

In one embodiment, v=0, v′=v″=1 and the precursor of the radical according to formula QV is tricarballylic acid.

In one embodiment, at least one of the Q′ is a radical according to formula QV,

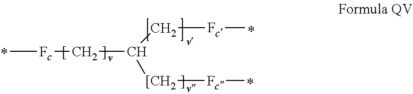

whose precursor is chosen in the group consisting of triamines.

In one embodiment, the precursor of the radical according to formula QV is a triamine chosen in the group consisting of (2-(amino methyl)propane-1,3-diamine).

In one embodiment, v=v′=v″=1 and the precursor of the radical according to formula V is (2-(aminoethyl)propane-1,3-diamine).

In one embodiment, at least one of the Q′ is a radical according to formula QVI,

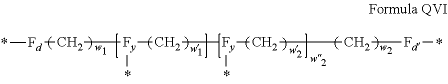

whose precursor is a triamine.

In one embodiment, w″₂=0 and the precursor of the radical according to formula QVI is a triamine chosen in the group consisting of spermidine, norspermidine, and diethylenetriamine and bis(hexamethylene)triamine.

In one embodiment, w″₂=0 and the precursor of the radical according to formula QVI is spermidine.

In one embodiment, w″₂=0 and the precursor of the radical according to formula QVI is norspermidine.

In one embodiment, w″₂=0 and the precursor of the radical according to formula QVI is diethylenetriamine.

In one embodiment, w″₂=0 and the precursor of the radical according to formula QVI is bis(hexamethylene)triamine.

In one embodiment, w″₂=1 and the precursor of the radical according to formula QVI is tetramine.

In one embodiment, w″₂=1 and the precursor of the radical according to formula QVI is a tetramine chosen in the group consisting of spermine and triethylenetetramine.

In one embodiment, w″₂=1 and the precursor of the radical according to formula QVI is spermine.

In one embodiment, w″₂=1 and the precursor of the radical according to formula QVI is triethylenetetramine. In one embodiment, the PLG are bound to Fx with Fx=—NH— or to Fy by at least one carbonyl function of the PLG.

In one embodiment, the PLG are bound to Fx with Fx=—NH— or to Fy by at least one carbonyl function that is not in the C-terminal position of the PLG.

In one embodiment, the PLG are bound to Fx with Fx=—NH— or to Fy by the carbonyl function in the C-terminal position of the PLG.

In one embodiment, the PLG are bound to Fx with Fx=—NH— or by the carbonyl function in the C-terminal position of the PLG.

In one embodiment, the PLG are bound to Fx with Fx=Fy by the carbonyl function in the C-terminal position of the PLG.

In one embodiment, the Hy are bound to Fx with Fx=—NH— or to Fy by a carbonyl function of Hy borne by GpR, GpA, GpG, GpH, GpL or GpC.

In one embodiment, the Hy are bound to Fy by a carbonyl function of Hy borne by GpR, GpA, GpG, GpH, GpL or GpC.

In one embodiment, the Hy are bound to Fx with Fx=—NH— by a carbonyl function of Hy borne by GpR, GpA, GpG, GpH, GpL or GpC.

In one embodiment, the PLG are bound to Fx with Fx=—CO— by the nitrogen atom in the N-terminal position of the PLG.

In one embodiment, the Hy are bound to Fx with Fx=—CO— by a nitrogen atom of Hy borne by GpR, GpA, GpG, GpL or GpH.

In one embodiment, the composition according to the invention is characterized in that n+m is comprised from 10 to 200.

In one embodiment, the composition according to the invention is characterized in that n+m is comprised from 15 to 150.

In one embodiment, the composition according to the invention is characterized in that n+m is comprised from 15 to 100.

In one embodiment, the composition according to the invention is characterized in that n+m is comprised from 15 to 80.

In one embodiment, the composition according to the invention is characterized in that n+m is comprised from 15 to 65.

In one embodiment, the composition according to the invention is characterized in that n+m is comprised from 20 to 60.

In one embodiment, the composition according to the invention is characterized in that n+m is comprised from 20 to 50.

In one embodiment, the composition according to the invention is characterized in that n+m is comprised of from 20 to 40.

The invention also relates to the co-polyamino acid bearing carboxylate charges and hydrophobic radicals Hy, said co-polyamino acid consisting glutamic or aspartic units and said hydrophobic radicals Hy being chosen among the radicals according to formula X as defined below:

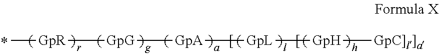

in which
GpR is chosen among the radicals according to formulas VII, VII′ or VII″:

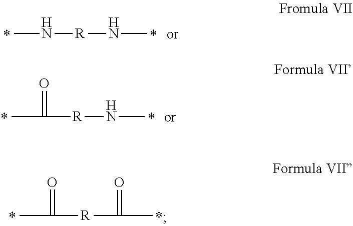

GpG and GpH, which are identical or different, are chosen among the radicals according to formulas XI or XI′:

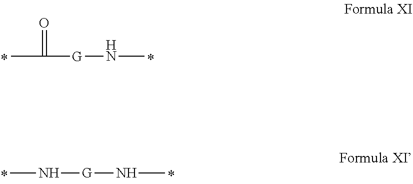

GpA is chosen among the radicals according to formula VIII

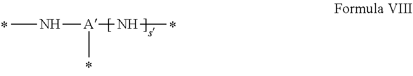

In which A′ is chosen among the radicals according to formulas VIII′, VIII″ or VIII′″

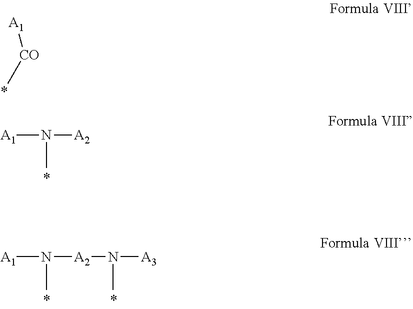

-GpL is chosen among the radicals according to formula XII

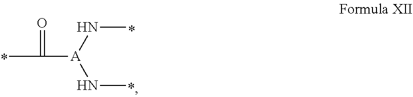

GpC is a radical according to formula IX:

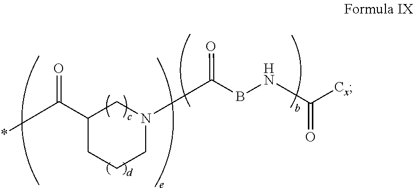

the * indicates the attachment sites of the different groups bound by amide functions;
a is an integer equal to 0 or 1 and a′=1 if a=0 and a′=1, 2 or 3 if a=1;
a′ is an integer equal to 1, to 2 or 3;
b is an integer equal to 0 or 1;
c is an integer equal to 0 or 1, and if c is equal to 0 then d is equal to 1 or 2;
d is an integer equal to 0, 1 or 2;
e is an integer equal to 0 or 1;
g is an integer equal to 0, 1, 2, 3, 4, 5 or 6;
h is an integer equal to 0, to 1, to 2, to 3 to 4 to 5 or to 6, and at least one of g, h or
l is different from 0;
l is an integer equal to 0 or 1 and l′=1 if l=0 and l′=2 if l=1;
r is an integer equal to 0, 1 or to 2, and
s′ is an integer equal to 0 or 1;
A, A₁, A₂ and A₃, which are identical or different, are linear or branched alkyl radicals comprising from 1 to 8 carbon atoms and optionally substituted by a radical from a saturated, unsaturated or aromatic ring;
B is a radical chosen in the group consisting of an unsubstituted ether or polyether radical comprising from 4 to 14 carbon atoms and from 1 to 5 oxygen atoms or a linear or branched alkyl radical, optionally comprising an aromatic ring, comprising from 1 to 9 carbon atoms;
Cₓ, is a linear or branched monovalent alkyl radical, optionally comprising a cyclic part, in which x indicates the number of carbon atoms and:
   When the hydrophobic radical -Hy bears 1 -GpC, then 9≤x≤25,
   When the hydrophobic radical -Hy bears 2 -GpC, then 9≤x≤15,
   When the hydrophobic radical -Hy bears 3 -GpC, then 7≤x≤13,
   When the hydrophobic radical -Hy bears 4 -GpC, then 7≤x≤11,
   When the hydrophobic radical -Hy bears at least 5 -GpC, then 6≤x≤11,
G is a linear or branched divalent alkyl radical of 1 to 8 carbon atoms, said alkyl radical bearing one or more free carboxylic acid function(s),
R is a radical chosen in the group consisting of a linear or branched divalent alkyl radical comprising from 1 to 12 carbon atoms, a linear or branched divalent alkyl radical comprising from 1 to 12 carbon atoms bearing one or more —CONH₂ functions or an unsubstituted ether or polyether radical comprising from 4 to 14 carbon atoms and from 1 to 5 oxygen atoms,
The hydrophobic radical(s) -Hy according to formula X being bound to PLG:
   via a covalent bond between a carbonyl of the hydrophobic radical -Hy and a nitrogen atom borne by the PLG thus forming an amide function resulting from the reaction of an amine function borne by the PLG and an acid function borne by the precursor -Hy′ of the hydrophobic radical -Hy, and
   via a covalent bond between a nitrogen atom from the hydrophobic radical -Hy and a carbonyl borne by the PLG thus forming an amide function resulting from the reaction of an amine function of the precursor -Hy′ of the hydrophobic radical -Hy and an acid function borne by the PLG,
the ratio M between the number of hydrophobic radicals and the number of glutamic or aspartic units being between 0<M≤0.5;
when several hydrophobic radicals are borne by a co-polyamino acid then they are identical or different,
the degree of polymerization DP in glutamic or aspartic units for the PLG chains is from 5 to 250;
the free carboxylic acid functions being in the form of an alkali metal salt chosen in the group consisting of Na⁺ and K⁺.

The invention also relates to the precursor Hy′ of the hydrophobic radical -Hy according to Formula X′ as defined below:

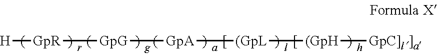

in which

GpR is chosen among the radicals according to formulas VII, VII′ or VII″:

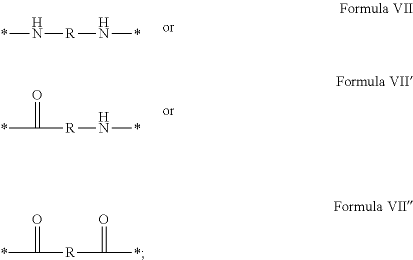

GpG and GpH, which are identical or different, are chosen among the radicals according to formulas XI or XI′:

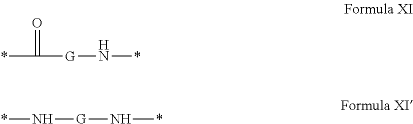

GpA is chosen among the radicals according to formula VIII

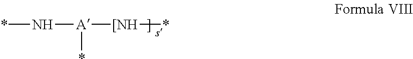

In which A′ is chosen among the radicals according to formulas VIII′, VIII″ or VIII′″

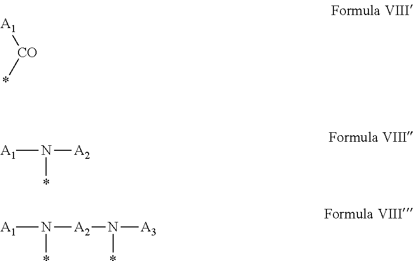

GpL is chosen among the radicals according to formula XII

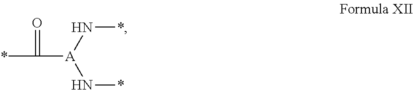

GpC is a radical according to formula IX:

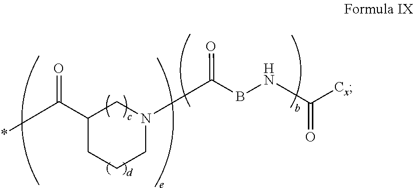

the * indicates the attachment sites of the different groups bound by amide functions;
a is an integer equal to 0 or 1 and a′=1 if a=0 and a′=1, 2 or 3 if a=1;
a′ is an integer equal to 1, to 2 or 3;
b is an integer equal to 0 or 1;
c is an integer equal to 0 or 1, and if c is equal to 0 then d is equal to 1 or 2;
d is an integer equal to 0, 1 or 2;
e is an integer equal to 0 or 1;
g is an integer equal to 0, 1, 2, 3, 4, 5 or 6;
h is an integer equal to 0, to 1, to 2, to 3 to 4 to 5 or to 6, and at least one of g, h or
   l is different from 0;
l is an integer equal to 0 or 1 and l′=1 if 1=0 and l′=2 if l=1;
r is an integer equal to 0, 1 or to 2, and
s′ is an integer equal to 0 or 1;
A, A₁, A₂ and A₃, which are identical or different, are linear or branched alkyl radicals comprising from 1 to 8 carbon atoms and optionally substituted by a radical from a saturated, unsaturated or aromatic ring;
B is a radical chosen in the group consisting of an unsubstituted ether or polyether radical comprising from 4 to 14 carbon atoms and from 1 to 5 oxygen atoms or a linear or branched alkyl radical, optionally comprising an aromatic ring, comprising from 1 to 9 carbon atoms;
Cₓ is a linear or branched monovalent alkyl radical, optionally comprising a cyclic part, in which x indicates the number of carbon atoms and:
   When the hydrophobic radical -Hy bears 1 -GpC, then 9≤x≤25,
   When the hydrophobic radical -Hy bears 2 -GpC, then 9≤x≤15,
   When the hydrophobic radical -Hy bears 3 -GpC, then 7≤x≤13,
   When the hydrophobic radical -Hy bears 4 -GpC, then 7≤x≤11,
   When the hydrophobic radical -Hy bears at least 5 -GpC, then 6≤x≤11,
G is a linear or branched divalent alkyl radical of 1 to 8 carbon atoms, said alkyl radical bearing one or more free carboxylic acid function(s),
R is a radical chosen in the group consisting of a linear or branched divalent alkyl radical comprising from 1 to 12 carbon atoms, a linear or branched divalent alkyl radical comprising from 1 to 12 carbon atoms bearing one or more —CONH₂ functions or an unsubstituted ether or polyether radical comprising from 4 to 14 carbon atoms and from 1 to 5 oxygen atoms,
The hydrophobic radical(s) -Hy according to formula X being bound to PLG:
   via a covalent bond between a carbonyl of the hydrophobic radical -Hy and a nitrogen atom borne by the PLG thus forming an amide function resulting from the reaction of an amine function borne by the PLG and an acid function borne by the precursor -Hy′ of the hydrophobic radical -Hy, and
   via a covalent bond between a nitrogen atom from the hydrophobic radical -Hy and a carbonyl borne by the PLG thus forming an amide function resulting from the reaction of an amine function of the precursor -Hy′ of the hydrophobic radical -Hy and an acid function borne by the PLG,
the ratio M between the number of hydrophobic radicals and the number of glutamic or aspartic units being between 0<M≤0.5;
when several hydrophobic radicals are borne by a co-polyamino acid then they are identical or different,
the free carboxylic acid functions being in the form of an alkali metal salt chosen in the group consisting of Na⁺ and K⁺.

In one embodiment, the composition according to the invention is characterized in that the co-polyamino acid is derived from a polyamino acid obtained by polymerization.

In one embodiment, the composition according to the invention is characterized in that the co-polyamino acid is derived from a polyamino acid obtained by polymerization by opening a ring derivative of an N-carboxy anhydride glutamic acid or a derivative of an aspartic acid N-carboxy anhydride.

In one embodiment, the composition according to the invention is characterized in that the co-polyamino acid is derived from a polyamino acid obtained by polymerization of a derivative of a glutamic acid N-carboxy anhydride or a derivative of an aspartic acid N-carboxy anhydride as described in the Review Article of Adv. Polym. Sci. 2006, 202, 1-18 (Deming, T. J.).

In one embodiment, the composition according to the invention is characterized in that the co-polyamino acid is derived from a polyamino acid obtained by polymerization of a derivative of a glutamic acid N-carboxy anhydride.

In one embodiment, the composition according to the invention is characterized in that the co-polyamino acid is derived from a polyamino acid obtained by polymerization of a derivative of a glutamic acid N-carboxy anhydride chosen from a group consisting of methyl poly-glutamate N-carboxy anhydride (GluOMe-NCA), benzyl polyglutamate N-carboxy anhydride (GluOBzl-NCA) and t-butyl polyglutamate N-carboxy anhydride (GluOtBu-NCA)

In one embodiment, the glutamic acid N-carboxy anhydride derivative is methyl poly-L-glutamate N-carboxy anhydride (L-GluOMe-NCA).

In one embodiment, the glutamic acid N-carboxy anhydride derivative is methyl poly-L-glutamate N-carboxy anhydride (L-GluOMe-NCA).

In one embodiment, the composition according to the invention is characterized in that the co-polyamino acid is derived from a polyamino acid obtained by polymerization of a derivative of a glutamic acid N-carboxy anhydride or a derivative of an aspartic acid N-carboxy anhydride by using a transition metal organometallic complex as an initiator as described in the 1997 publication of Nature, 390, 386-389 (Deming, T. J.).

In one embodiment, the composition according to the invention is characterized in that the co-polyamino acid is derived from a polyamino acid obtained by polymerization of a derivative of a glutamic acid N-carboxy anhydride or a derivative of an aspartic acid N-carboxy anhydride by using ammonia or a primary amine as an initiator as described in patent FR 2,801,226 (Touraud, F., et al.) and references cited therein.

In one embodiment, the composition according to the invention is characterized in that the co-polyamino acid is derived from a polyamino acid obtained by polymerization of a derivative of a glutamic acid N-carboxy anhydride or of an aspartic acid N-carboxy anhydride by using hexamethyldisilazane as an initiator as described in J. Am. Chem. Soc. 2007, 129, 14114-14115 (Lu H., et al.) or a silylated amine as described in publication J. Am. Chem. Soc. 2008, 130, 12562-12563 (Lu H., et al.).

In one embodiment, the composition according to the invention is characterized in that the process for the synthesis of the polyamino acid obtained by polymerization of a glutamic acid N-carboxy anhydride derivative or an aspartic acid N-carboxy anhydride derivative from which the co-polyamino acid is derived comprises a hydrolysis step of the ester functions.

In one embodiment, this ester function hydrolysis step may consist of hydrolysis in an acidic medium or hydrolysis in a basic medium or may be carried out by hydrogenation.

In one embodiment, this ester group hydrolysis step is a hydrolysis in an acidic medium.

In one embodiment, this ester group hydrolysis step is performed by hydrogenation.

In one embodiment, the composition according to the invention is characterized in that the co-polyamino acid is derived from a polyamino acid obtained by depolymerization of a polyamino acid of a higher molecular weight.

In one embodiment, the composition according to the invention is characterized in that the co-polyamino acid is derived from a polyamino acid obtained by enzymatic depolymerization of a polyamino acid of a higher molecular weight.

In one embodiment, the composition according to the invention is characterized in that the co-polyamino acid is derived from a polyamino acid obtained by chemical depolymerization of a polyamino acid of a higher molecular weight.

In one embodiment, the composition according to the invention is characterized in that the co-polyamino acid is derived from a polyamino acid obtained by enzymatic and chemical depolymerization of a polyamino acid of a higher molecular weight.

In one embodiment, the composition according to the invention is characterized in that the co-polyamino acid is derived from a polyamino acid obtained by depolymerization of a polyamino acid of a higher molecular weight chosen in the group consisting of sodium polyglutamate and sodium polyaspartate.

In one embodiment, the composition according to the invention is characterized in that the co-polyamino acid is derived from a polyamino acid obtained by depolymerization of a sodium polyglutamate of a higher molecular weight.

In one embodiment, the composition according to the invention is characterized in that the co-polyamino acid is derived from a polyamino acid obtained by depolymerization of a sodium polyglutamate of a higher molecular weight.

In one embodiment, the composition according to the invention is characterized in that the co-polyamino acid is obtained by grafting a hydrophobic group to a poly-L-glutamic acid or poly-L-aspartic acid using amide bond-forming methods well known to those skilled in the art.

In one embodiment, the composition according to the invention is characterized in that the co-polyamino acid is obtained by grafting a hydrophobic group to a poly-L-glutamic acid or poly-L-aspartic acid using amide bond-forming methods for peptide synthesis.

In one embodiment, the composition according to the invention is characterized in that the co-polyamino acid is obtained by grafting a hydrophobic group to a poly-L-glutamic acid or poly-L-aspartic acid as described in patent FR 2,840,614 (Chan, Y. P, et al.).

m-Cresol and Preservatives—See Further Below to Remove the Preservative Portion

According to one particular embodiment, the composition comprises a concentration of m-cresol lower than or equal to 29 mM

According to one particular embodiment, the composition comprises a concentration of m-cresol lower than or equal to 28 mM

According to one particular embodiment, the composition comprises a concentration of m-cresol lower than or equal to 25 mM

According to one particular embodiment, the composition comprises a concentration of m-cresol lower than or equal to 23 mM

According to one particular embodiment, the composition comprises a concentration of m-cresol lower than or equal to 20 mM

According to one particular embodiment, the composition comprises a concentration of m-cresol lower than or equal to 19 mM

According to one particular embodiment, the composition comprises a concentration of m-cresol lower than or equal to 17 mM

According to one particular embodiment, the composition comprises a concentration of m-cresol lower than or equal to 16 mM

According to one particular embodiment, the composition comprises a concentration of m-cresol lower than or equal to 14 mM.

According to one particular embodiment, the composition comprises a concentration of m-cresol lower than or equal to 12 mM.

According to one particular embodiment, the composition comprises a concentration of m-cresol lower than or equal to 10 mM.

According to one particular embodiment, the composition comprises a concentration of m-cresol lower than or equal to 8 mM.

According to one particular embodiment, the composition comprises a concentration of m-cresol a concentration equal to 1, 2, 3, 4, 5, 7, 8, 9, 10, 11, 12, 13, 14, 15, 16, 17, 18, 19, 20, 21, 22, 23, 24, 25, 26, 27, 28, 29 or 30 mM.

According to one particular embodiment, the composition comprises a concentration of m-cresol ranging from 5 to 30 mM.

According to one particular embodiment, the composition comprises a concentration of m-cresol ranging from 10 to 30 mM.

According to one particular embodiment, the composition comprises a concentration of m-cresol ranging from 15 to 30 mM.

According to one particular embodiment, the composition comprises a concentration of m-cresol ranging from 5 to 25 mM.

According to one particular embodiment, the composition comprises a concentration of m-cresol ranging from 10 to 25 mM.

According to one particular embodiment, the composition comprises a concentration of m-cresol ranging from 15 to 25 mM.

According to one particular embodiment, the composition comprises a concentration of m-cresol ranging from 5 to 20 mM.

According to one particular embodiment, the composition comprises a concentration of m-cresol ranging from 10 to 20 mM.

According to one particular embodiment, the composition comprises a concentration of m-cresol ranging from 15 to 20 mM.

In one embodiment, the composition comprises phenol.

According to one embodiment, the composition comprises a concentration of phenol ranging from 10 to 100 mM.

According to one embodiment, the composition comprises a concentration of phenol ranging from 10 to 50 mM.

According to one embodiment, the composition comprises a concentration of phenol ranging from 10 to 25 mM.

According to one embodiment, the composition comprises a concentration in m-cresol lower than or equal to 30 mM and a concentration in phenol ranging from 10 to 50 mM.

According to one embodiment, the composition comprises a concentration in m-cresol ranging from 10 to 30 mM and a concentration in phenol ranging from 10 to 50 mM.

According to one embodiment, the composition comprises a concentration in m-cresol ranging from 10 to 30 mM and a concentration in phenol ranging from 10 to 30 mM.

According to one embodiment, the composition comprises a concentration in m-cresol ranging from 10 to 20 mM and a concentration in phenol ranging from 10 to 50 mM.

According to one embodiment, the composition comprises a concentration in m-cresol ranging from 10 to 20 mM and a concentration in phenol ranging from 10 to 30 mM.

Advantageously, the addition of phenol does not interfere with the improvement brought by the decrease in the concentration of m-cresol, while making it possible to obtain a satisfying preservative activity.

According to one embodiment, the composition has an antimicrobial preservation level that complies with the requirements for placing a medication on the market.

According to one embodiment, the composition may comprise benzyl alcohol.

### Salt Effect—Zn and NaCl

In one embodiment, the composition comprises Zn ions.

According to one embodiment, the composition comprises a concentration of Zn ions from 0.2 to 20 mM.

According to one embodiment, the composition comprises a concentration of Zn ions from 0.2 to 10 mM.

According to one embodiment, the composition comprises a concentration of Zn ions from 0.2 to 5 mM.

In one embodiment, the composition comprises from 0.2 to 2 mM of zinc.

According to one embodiment, the composition comprises a concentration of Zn ions from 0.5 to 20 mM.

According to one embodiment, the composition comprises a concentration of Zn ions from 0.5 to 10 mM.

According to one embodiment, the composition comprises a concentration of Zn ions from 0.5 to 5 mM.

According to one embodiment, the composition comprises a concentration of Zn ions from 0.5 to 2 mM.

According to one embodiment, the composition comprises a concentration of Zn ions from 1 to 20 mM.

According to one embodiment, the composition comprises a concentration of Zn ions from 1 to 10 mM.

According to one embodiment, the composition comprises a concentration of Zn ions from 1 to 5 mM.

According to one embodiment, the composition comprises a concentration of Zn ions from 1 to 2 mM.

In one embodiment, the composition comprises NaCl.

In one embodiment, NaCl is present in a concentration ranging from 2 to 25 mM.

In one embodiment, NaCl is present in a concentration ranging from 2.5 to 20 mM.

In one embodiment, NaCl is present in a concentration ranging from 4 to 15 mM.

In one embodiment, NaCl is present in a concentration ranging from 5 to 10 mM.

In one embodiment, the composition comprises Zn and NaCl ions.

According to one embodiment, the composition comprises a concentration in Zn ions from 1 to 5 mM and of NaCl in a concentration ranging from 2 to 25 mM.

According to one embodiment, the composition comprises a concentration in Zn ions from 1 to 5 mM and of NaCl in a concentration ranging from 2.5 to 20 mM.

According to one embodiment, the composition comprises a concentration in Zn ions from 1 to 5 mM and of NaCl in a concentration ranging from 4 to 15 mM.

According to one embodiment, the composition comprises a concentration in Zn ions from 1 to 5 mM and of NaCl in a concentration ranging from 5 to 10 mM.

### M-Cresol and Salts Combination

According to one embodiment, the composition includes a concentration in NaCl from 4 to 15 mM, a concentration in Zn ions from 0.2 to 2 mM and a concentration in m-cresol lower than 28 mM.

According to one embodiment, the composition includes a concentration in NaCl from 4 to 15 mM, a concentration in Zn ions from 0.5 to 2 mM and a concentration in m-cresol lower than 20 mM.

In the following, the units used for insulins are those recommended by the pharmacopoeia whose corresponding mg/ml values are given in the table below:

| | EP Pharmacopoeia 8.0 | US Pharmacopoeia - USP38 |
|---|---|---|
| Insulin | (2014) | (2015) |
| Aspart | 1U = 0.0350 mg of | 1 USP = 0.0350 mg of aspart |
| | aspart insulin | insulin |
| Lispro | 1U = 0.0347 mg of | 1 USP = 0.0347 mg of insulin |
| | insulin lispro | lispro |
| Human | lUI = 0.0347 mg of | 1 USP = 0.0347 mg of human |
| | human insulin | insulin |
| Glargine | 1U = 0.0364 mg of | 1 USP = 0.0364 mg of insulin |
| | insulin glargine | glargine |
| Porcine | lUI = 0.0345 mg of | 1 USP = 0.0345 mg of porcine |
| | porcine insulin | insulin |
| Bovine | lUI = 0.0342 mg of | 1 USP = 0.0342 mg of bovine |
| | bovine insulin | insulin |

Basal insulin is understood to be insulin whose isoelectric point is from 5.8 to 8.5. It is an insoluble insulin at pH 7 and its duration of action is from 8 to 24 hours or greater in the standard diabetes models.

These basal insulins, whose isoelectric point is from 5.8 to 8.5 are recombinant insulins whose primary structure has been mainly modified by the introduction of basic amino acids such as Arginine or Lysine. They are described for example in the following patents, patent applications or publications WO 2003/053339, WO 2004/096854, U.S. Pat. Nos. 5,656,722 and 6,100,376, the content of which is incorporated by reference.

In one embodiment, the basal insulin whose isoelectric point is from 5.8 to 8.5 is insulin glargine. Insulin glargine is marketed under the brand Lantus® (100 U/ml) or Toujeo® (300 U/ml) by SANOFI.

In one embodiment, the basal insulin whose isoelectric point is from 5.8 to 8.5 is a biosimilar insulin glargine.

A biosimilar insulin glargine is in the process of being marketing under the brand Abasaglar® or Basaglar® by ELI LILLY.

In one embodiment, the compositions according to the invention comprise from 40 to 500 U/mL of basal insulin whose isoelectric point is from 5.8 to 8.5.

In one embodiment, the compositions according to the invention comprise 40 U/mL of basal insulin whose isoelectric point is from 5.8 to 8.5.

In one embodiment, the compositions according to the invention comprise 75 U/mL of basal insulin whose isoelectric point is from 5.8 to 8.5.

In one embodiment, the compositions according to the invention comprise 100 U/mL (or about 3.6 mg/mL) of basal insulin whose isoelectric point is from 5.8 to 8.5.

In one embodiment, the compositions according to the invention comprise 150 U/mL of basal insulin whose isoelectric point is from 5.8 to 8.5.

In one embodiment, the compositions according to the invention comprise 200 U/mL of basal insulin whose isoelectric point is from 5.8 to 8.5.

In one embodiment, the compositions according to the invention comprise 225 U/mL of basal insulin whose isoelectric point is from 5.8 to 8.5.

In one embodiment, the compositions according to the invention comprise 250 U/mL of basal insulin whose isoelectric point is from 5.8 to 8.5.

In one embodiment, the compositions according to the invention comprise 300 U/mL of basal insulin whose isoelectric point is from 5.8 to 8.5.

In one embodiment, the compositions according to the invention comprise 400 U/mL of basal insulin whose isoelectric point is from 5.8 to 8.5.

In one embodiment, the compositions according to the invention comprise 500 U/mL of basal insulin whose isoelectric point is from 5.8 to 8.5.

In one embodiment, the mass ratio between the basal insulin, whose isoelectric point is from 5.8 to 8.5, and the co-polyamino acid, i.e., co-polyamino acid/basal insulin, is from 0.2 to 8.

In one embodiment, the mass ratio is from 0.2 to 6.

In one embodiment, the mass ratio is from 0.2 to 5.

In one embodiment, the mass ratio is from 0.2 to 4.

In one embodiment, the mass ratio is from 0.2 to 3.

In one embodiment, the mass ratio is from 0.2 to 2.

In one embodiment, the mass ratio is from 0.2 to 1.

In one embodiment, the concentration of co-polyamino acids bearing carboxylate charges and hydrophobic radicals is at most 60 mg/ml.

In one embodiment, the concentration of co-polyamino acids bearing carboxylate charges and hydrophobic radicals is at most 40 mg/ml.

In one embodiment, the concentration of co-polyamino acids bearing carboxylate charges and hydrophobic radicals is at most 20 mg/ml.

In one embodiment, the concentration of co-polyamino acids bearing carboxylate charges and hydrophobic radicals is at most 10 mg/ml.

In one embodiment, the concentration of co-polyamino acids bearing carboxylate charges and hydrophobic radicals is at most 5 mg/ml.

In one embodiment, the concentration of co-polyamino acids bearing carboxylate charges and hydrophobic radicals is at most 2.5 mg/ml.

In one embodiment, the compositions according to the invention further comprise a prandial insulin. Prandial insulins are soluble at a pH of 7.

Prandial insulin is understood to be an insulin known to be fast or “regular”.

The so-called fast-acting prandial insulins are insulins that must meet the needs caused by the ingestion of proteins and carbohydrates during a meal, so they must act in less than 30 minutes.

In one embodiment, the so-called “regular” prandial insulin is human insulin.

In one embodiment, prandial insulin is a recombinant human insulin as described in the European Pharmacopoeia and the American Pharmacopoeia.

Human insulin is for example marketed under the brands Humulin® (ELI LILLY) and Novolin® (NOVO NORDISK).

The so-called fast-acting insulins are insulins which are obtained by recombination and whose primary structure has been modified to reduce their time of action.

In one embodiment, the so-called fast-acting prandial insulins are chosen in the group comprising insulin lispro (Humalog®), glulisine insulin (Apidra®) and aspart insulin (NovoLog®).

In one embodiment, the prandial insulin is insulin lispro.

In one embodiment, the prandial insulin is glulisine insulin.

In one embodiment, the prandial insulin is aspart insulin.

In one embodiment, the compositions according to the invention comprise from 60 to 800 U/mL of insulin with a combination of prandial and basal insulin whose isoelectric point is from 5.8 to 8.5.

In one embodiment, the compositions according to the invention comprise from 100 to 500 U/mL of insulin with a combination of prandial and basal insulin whose isoelectric point is from 5.8 to 8.5.

In one embodiment, the compositions according to the invention comprise a total of 800 U/mL of insulin with a combination of prandial and basal insulin whose isoelectric point is from 5.8 to 8.5.

In one embodiment, the compositions according to the invention comprise a total of 700 U/mL of insulin with a combination of prandial and basal insulin whose isoelectric point is from 5.8 to 8.5.

In one embodiment, the compositions according to the invention comprise a total of 600 U/mL of insulin with a combination of prandial and basal insulin whose isoelectric point is from 5.8 to 8.5.

In one embodiment, the compositions according to the invention comprise a total of 500 U/mL of insulin with a combination of prandial and basal insulin whose isoelectric point is from 5.8 to 8.5.

In one embodiment, the compositions according to the invention comprise a total of 400 U/mL of insulin with a combination of prandial and basal insulin whose isoelectric point is from 5.8 to 8.5.

In one embodiment, the compositions according to the invention comprise a total of 300 U/mL of insulin with a combination of prandial and basal insulin whose isoelectric point is from 5.8 to 8.5.

In one embodiment, the compositions according to the invention comprise a total of 266 U/mL of insulin with a combination of prandial and basal insulin whose isoelectric point is from 5.8 to 8.5.

In one embodiment, the compositions according to the invention comprise a total of 200 U/mL of insulin with a combination of prandial and basal insulin whose isoelectric point is from 5.8 to 8.5.

In one embodiment, the compositions according to the invention comprise a total of 100 U/mL of insulin with a combination of prandial and basal insulin whose isoelectric point is from 5.8 to 8.5.

The proportions between the basal insulin whose isoelectric point is from 5.8 to 8.5 and the prandial insulin are, for example, in percentages of 25/75, 30/70, 40/60, 50/50, 60/40, 63/37, 70/30, 75/25, 80/20, 83/17, 90/10 for formulations as described above comprising from 60 to 800 U/mL. However, any other proportion may be achieved.

In one embodiment, the basal insulin whose isoelectric point is from 5.8 to 8.5 and the prandial insulin are respectively present in the following concentrations (in U/ml) 75/25, 150/50, 200/66 or 300/100.

In one embodiment, the basal insulin whose isoelectric point is from 5.8 to 8.5 and the prandial insulin are respectively present in the following concentrations (in U/ml) 75/25.

In one embodiment, the basal insulin whose isoelectric point is from 5.8 to 8.5 and the prandial insulin are respectively present in the following concentrations (in U/ml) 150/50.

The ratio of hydrophobic radical to basal insulin is defined as the ratio of their respective molar concentrations: [Hy]/[basal insulin] (mol/mol) to obtain the expected performances, namely the solubilization of the basal insulin at a pH from 6.0 to 8.0, the precipitation of basal insulin and the stability of the compositions according to the invention.

The minimum measured value of the ratio hydrophobic radical to basal insulin [Hy]/[basal insulin], is the value at which the basal insulin is solubilized, since solubilization is the minimum effect to obtain; this solubilization is a condition for all the other technical effects that can only be observed if the basal insulin is solubilized at a pH from 6.0 to 8.0.

In the compositions according to the invention, the ratio of hydrophobic radical to basal insulin [Hy]/[basal insulin] may be greater than the minimum value determined by the solubilization limit.

In one embodiment, the ratio of hydrophobic radical to basal insulin [Hy]/[basal insulin]≤3.

In one embodiment, the ratio of hydrophobic radical to basal insulin [Hy]/[basal insulin]≤2.

In one embodiment, the ratio of hydrophobic radical to basal insulin [Hy]/[basal insulin]≤1.75.

In one embodiment, the ratio of hydrophobic radical to basal insulin [Hy]/[basal insulin]≤1.5.

In one embodiment, the ratio of hydrophobic radical to basal insulin [Hy]/[basal insulin]≤1.25.

In one embodiment, the ratio of hydrophobic radical to basal insulin [Hy]/[basal insulin]≤1.00.

In one embodiment, the ratio of hydrophobic radical to basal insulin [Hy]/[basal insulin]≤0.75.

In one embodiment, the ratio of hydrophobic radical to basal insulin [Hy]/[basal insulin]≤0.5.

In one embodiment, the ratio of hydrophobic radical to basal insulin [Hy]/[basal insulin]≤0.25.

In one embodiment, the compositions according to the invention further comprise a gut hormone.

By “gut hormones” is meant hormones chosen in the group consisting of GLP-1 RA (Glucagon-like peptide-1 receptor agonist) and the GIP (Glucose-dependent insulinotropic peptide), oxyntomodulin (a derivative of proglucagon), peptide YY, amylin, cholecystokinin, pancreatic polypeptide (PP), ghrelin and enterostatin, their analogues or derivatives and/or their pharmaceutically acceptable salts.

In one embodiment, the gut hormones are analogues or derivatives of GLP-1 RA chosen in the group consisting of exenatide or Byetta® (ASTRA-ZENECA), liraglutide or Victoza® (NOVO NORDISK), lixisenatide or Lyxumia® (SANOFI), albiglutide or Tanzeum® (GSK) or dulaglutide or Trulicity® (ELI LILLY & CO), their analogues or derivatives and their pharmaceutically acceptable salts.

In one embodiment, the gut hormone is pramlintide or Symlin® ®(ASTRA-ZENECA).

In one embodiment, the gut hormone is exenatide or Byetta®, its analogues or derivatives and their pharmaceutically acceptable salts.

In one embodiment, the gut hormone is liraglutide or Victoza®, its analogues or derivatives and their pharmaceutically acceptable salts.

In one embodiment, the gut hormone is lixisenatide or Lyxumia®, its analogues or derivatives and their pharmaceutically acceptable salts.

In one embodiment, the gut hormone is albiglutide or Tanzeum®, its analogues or derivatives and their pharmaceutically acceptable salts.

In one embodiment, the gut hormone is dulaglutide or Trulicity®, its analogues or derivatives and their pharmaceutically acceptable salts.

In one embodiment, the gut hormone is pramlintide or Symlin®, its analogues or derivatives and their pharmaceutically acceptable salts.

The term “analogue”, when used in reference to a peptide or protein, is meant a peptide or a protein, wherein one or more constituent amino acid residues have been substituted by other amino acid residues and/or wherein one or more constituent amino acid residues have been removed and/or wherein one or more constituent amino acid residues have been added. The percentage of homology allowed for the present definition of an analogue is 50%.

The term “derivative”, when used in reference to a peptide or a protein, is meant a peptide or a protein or an analogue chemically modified by a substituent that is not present in the peptide or the protein or the reference analogue, i.e., a peptide or protein that has been modified by the creation of covalent bonds, to introduce substituents.

In one embodiment, the substituent is chosen in the group consisting of fatty chains.

In one embodiment, the gut hormone concentration is comprised within a range from 0.01 to 100 mg/mL.

In one embodiment, the concentration of exenatide, its analogs or derivatives and their pharmaceutically acceptable salts is within the range from 0.04 to 0.5 mg/mL.

In one embodiment, the concentration of liraglutide, its analogs or derivatives and their pharmaceutically acceptable salts is comprised within the range from 1 to 10 mg/mL.

In one embodiment, the concentration of lixisenatide, its analogs or derivatives and their pharmaceutically acceptable salts is comprised within the range from 0.01 to 1 mg/mL.

In one embodiment, the concentration of albiglutide, its analogs or derivatives and their pharmaceutically acceptable salts is comprised within the range from 5 to 100 mg/mL.

In one embodiment, the concentration of dulaglutide, its analogs or derivatives and their pharmaceutically acceptable salts is comprised within the range from 0.1 to 10 mg/mL.

In one embodiment, the concentration of pramlintide, its analogs or derivatives and their pharmaceutically acceptable salts is comprised within the range from 0.1 to 5 mg/mL.

In one embodiment, the compositions according to the invention are produced by mixing commercial solutions of basal insulin whose isoelectric point is from 5.8 to 8.5 and commercial solutions of GLP-1 RA, GLP-1 RA analogs or derivatives in volume ratios ranging from 10/90 to 90/10.

In one embodiment, the composition according to the invention comprises a daily dose of basal insulin and a daily dose of gut hormone.

In one embodiment, the compositions according to the invention comprise from 40 U/mL to 500 U/mL of basal insulin whose isoelectric point is from 5.8 to 8.5 and from 0.05 to 0.5 mg/mL of exenatide.

In one embodiment, the compositions according to the invention comprise from 40 U/mL to 500 U/mL of basal insulin whose isoelectric point is from 5.8 to 8.5 and from 1 to 10 mg/mL of liraglutide.

In one embodiment, the compositions according to the invention comprise from 40 U/mL to 500 U/mL of basal insulin whose isoelectric point is from 5.8 to 8.5 and from 0.01 to 1 mg/mL of lixisenatide.

In one embodiment, the compositions according to the invention comprise from 40 U/mL to 500 U/mL of basal insulin whose isoelectric point is from 5.8 to 8.5 and from 5 to 100 mg/mL of albiglutide.

In one embodiment, the compositions according to the invention comprise from 40 U/mL to 500 U/mL of basal insulin whose isoelectric point is from 5.8 to 8.5 and from 0.1 to 10 mg/mL of dulaglutide.

In one embodiment, the compositions according to the invention comprise 500 U/mL of basal insulin whose isoelectric point is from 5.8 to 8.5 and, from 0.04 to 0.5 mg/ml of exenatide.

In one embodiment, the compositions according to the invention comprise 500 U/mL of basal insulin whose isoelectric point is from 5.8 to 8.5 and, from 1 to 10 mg/ml of liraglutide.

In one embodiment, the compositions according to the invention comprise 500 U/mL of basal insulin whose isoelectric point is from 5.8 to 8.5 and, from 0.01 to 1 mg/mL of lixisenatide.

In one embodiment, the compositions according to the invention comprise 500 U/mL of basal insulin whose isoelectric point is from 5.8 to 8.5 and, from 5 to 100 mg/ml of albiglutide.

In one embodiment, the compositions according to the invention comprise 500 U/mL of basal insulin whose isoelectric point is from 5.8 to 8.5 and, from 0.1 to 10 mg/mL of dulaglutide.

In one embodiment, the compositions according to the invention comprise 400 U/mL of basal insulin whose isoelectric point is from 5.8 to 8.5 and, from 0.04 to 0.5 mg/ml of exenatide.

In one embodiment, the compositions according to the invention comprise 400 U/mL of basal insulin whose isoelectric point is from 5.8 to 8.5 and, from 1 to 10 mg/ml of liraglutide.

In one embodiment, the compositions according to the invention comprise 400 U/mL of basal insulin whose isoelectric point is from 5.8 to 8.5 and, from 0.01 to 1 mg/mL of lixisenatide.

In one embodiment, the compositions according to the invention comprise 400 U/mL of basal insulin whose isoelectric point is from 5.8 to 8.5 and, from 5 to 100 mg/ml of albiglutide.

In one embodiment, the compositions according to the invention comprise 400 U/mL of basal insulin whose isoelectric point is from 5.8 to 8.5 and, from 0.1 to 10 mg/mL of dulaglutide.

In one embodiment, the compositions according to the invention comprise 300 U/mL of basal insulin whose isoelectric point is from 5.8 to 8.5 and, from 0.04 to 0.5 mg/ml of exenatide.

In one embodiment, the compositions according to the invention comprise 300 U/mL of basal insulin whose isoelectric point is from 5.8 to 8.5 and, from 1 to 10 mg/ml of liraglutide.

In one embodiment, the compositions according to the invention comprise 300 U/mL of basal insulin whose isoelectric point is from 5.8 to 8.5 and, from 0.01 to 1 mg/mL of lixisenatide.

In one embodiment, the compositions according to the invention comprise 300 U/mL of basal insulin whose isoelectric point is from 5.8 to 8.5 and, from 5 to 100 mg/ml of albiglutide.

In one embodiment, the compositions according to the invention comprise 300 U/mL of basal insulin whose isoelectric point is from 5.8 to 8.5 and, from 0.1 to 10 mg/mL of dulaglutide.

In one embodiment, the compositions according to the invention comprise 225 U/mL of basal insulin whose isoelectric point is from 5.8 to 8.5 and, from 0.04 to 0.5 mg/ml of exenatide.

In one embodiment, the compositions according to the invention comprise 225 U/mL of basal insulin whose isoelectric point is from 5.8 to 8.5 and, from 1 to 10 mg/ml of liraglutide.

In one embodiment, the compositions according to the invention comprise 225 U/mL of basal insulin whose isoelectric point is from 5.8 to 8.5 and, from 0.01 to 1 mg/mL of lixisenatide.

In one embodiment, the compositions according to the invention comprise 225 U/mL of basal insulin whose isoelectric point is from 5.8 to 8.5 and, from 5 to 100 mg/ml of albiglutide.

In one embodiment, the compositions according to the invention comprise 225 U/mL of basal insulin whose isoelectric point is from 5.8 to 8.5 and, from 0.1 to 10 mg/mL of dulaglutide.

In one embodiment, the compositions according to the invention comprise 200 U/mL of basal insulin whose isoelectric point is from 5.8 to 8.5 and, from 0.04 to 0.5 mg/ml of exenatide.

In one embodiment, the compositions according to the invention comprise 200 U/mL of basal insulin whose isoelectric point is from 5.8 to 8.5 and, from 1 to 10 mg/ml of liraglutide.

In one embodiment, the compositions according to the invention comprise 200 U/mL of basal insulin whose isoelectric point is from 5.8 to 8.5 and, from 0.01 to 1 mg/mL of lixisenatide.

In one embodiment, the compositions according to the invention comprise 200 U/mL of basal insulin whose isoelectric point is from 5.8 to 8.5 and, from 5 to 100 mg/ml of albiglutide.

In one embodiment, the compositions according to the invention comprise 200 U/mL of basal insulin whose isoelectric point is from 5.8 to 8.5 and, from 0.1 to 10 mg/mL of dulaglutide.

In one embodiment, the compositions according to the invention comprise 100 U/mL (or about 3.6 mg/mL) of basal insulin whose isoelectric point is from 5.8 to 8.5 and, from 0.04 to 0.5 mg/ml of exenatide.

In one embodiment, the compositions according to the invention comprise 100 U/mL (or about 3.6 mg/mL) of basal insulin whose isoelectric point is from 5.8 to 8.5 and, from 1 to 10 mg/mL of liraglutide.

In one embodiment, the compositions according to the invention comprise 100 U/mL (or about 3.6 mg/mL) of basal insulin whose isoelectric point is from 5.8 to 8.5 and, from 0.01 to 1 mg/mL of lixisenatide.

In one embodiment, the compositions according to the invention comprise 100 U/mL of basal insulin whose isoelectric point is from 5.8 to 8.5 and, from 5 to 100 mg/ml of albiglutide.

In one embodiment, the compositions according to the invention comprise 100 U/mL of basal insulin whose isoelectric point is from 5.8 to 8.5 and, from 0.1 to 10 mg/mL of dulaglutide.

In one embodiment, the compositions according to the invention comprise 40 U/mL of basal insulin whose isoelectric point is from 5.8 to 8.5 and, from 0.04 to 0.5 mg/ml of exenatide.

In one embodiment, the compositions according to the invention comprise 40 U/mL of basal insulin whose isoelectric point is from 5.8 to 8.5 and, from 1 to 10 mg/ml of liraglutide.

In one embodiment, the compositions according to the invention comprise 40 U/mL of basal insulin whose isoelectric point is from 5.8 to 8.5 and, from 0.01 to 1 mg/mL of lixisenatide.

In one embodiment, the compositions according to the invention comprise 40 U/mL of basal insulin whose isoelectric point is from 5.8 to 8.5 and, from 5 to 100 mg/ml of albiglutide.

In one embodiment, the compositions according to the invention comprise 40 U/mL of basal insulin whose isoelectric point is from 5.8 to 8.5 and, from 0.1 to 10 mg/mL of dulaglutide.

The invention also relates to compositions which further comprise ionic species, said ionic species making it possible to improve the stability of the compositions.

The invention also relates to the use of ionic species chosen in the group of anions, cations and/or zwitterions to improve the physicochemical stability of the compositions.

In one embodiment, the ionic species comprise less than 10 carbon atoms.

Said ionic species are chosen from a group of anions, cations and/or zwitterions. By zwitterion is meant a species bearing at least one positive charge and at least one negative charge on two non-adjacent atoms.

Said ionic species are used alone or in a mixture and preferably in a mixture.

In one embodiment, the anions are chosen from organic anions.

In one embodiment, the organic anions comprise less than 10 carbon atoms.

In one embodiment, the organic anions are chosen from a group consisting of acetate, citrate and succinate.

In one embodiment, the anions are chosen from anions of mineral origin.

In one embodiment, the anions of mineral origin are chosen in the group consisting of sulphates, phosphates and halides, especially chlorides.

In one embodiment, the cations are chosen from organic cations.

In one embodiment, the organic cations comprise less than 10 carbon atoms.

In one embodiment, the organic cations are chosen in the group consisting of ammoniums, for example 2-Amino-2-(hydroxymethyl) propane-1,3-diol wherein the amine is in ammonium form.

In one embodiment, the cations are chosen from cations of mineral origin.

In one embodiment, the cations of mineral origin are chosen in the group consisting of zinc, in particular Zn2+ and alkali metals, in particular Na+ and K+,

In one embodiment, the zwitterions are chosen from zwitterions of organic origin.

In one embodiment, the zwitterions of organic origin are chosen from amino acids.

In one embodiment, the amino acids are chosen from aliphatic amino acids in the group consisting of glycine, alanine, valine, isoleucine and leucine.

In one embodiment, the amino acids are chosen from cyclic amino acids in the group consisting of proline.

In one embodiment the amino acids are chosen from hydroxylated or sulfur amino acids in the group consisting of cysteine, serine, threonine, and methionine.

In one embodiment, the amino acids are chosen from aromatic amino acids in the group consisting of phenylalanine, tyrosine and tryptophan.

In one embodiment, the amino acids are chosen from amino acids whose carboxyl function of the side chain is amidified in the group consisting of asparagine and glutamine.

In one embodiment, the zwitterions of organic origin are chosen in the group consisting of amino acids having an uncharged side chain.

In one embodiment, the zwitterions of organic origin are chosen in the group consisting of amino diacids or acidic amino acids.

In one embodiment, the amino diacids are chosen in the group consisting of glutamic acid and aspartic acid, optionally in the form of salts.

In one embodiment, the zwitterions of organic origin are chosen in the group consisting of basic or so-called “cationic” amino acids.

In one embodiment, the so-called “cationic” amino acids are chosen from arginine, histidine and lysine, in particular arginine and lysine.

In particular, the zwitterions comprise as many negative charges as positive charges and therefore a nil overall charge at the isoelectric point and/or at a pH from 6.0 to 8.0.

Said ionic species are introduced into the compositions in the form of salts. The introduction of these can be in solid form before dissolution in the compositions, or in the form of a solution, in particular of a concentrated solution.

For example, the cations of mineral origin are provided in the form of salts chosen from sodium chloride, zinc chloride, sodium phosphate, sodium sulfate, etc.

For example, anions of organic origin are provided in the form of salts chosen from sodium or potassium citrate, sodium acetate.

For example, the amino acids are added in the form of salts chosen from arginine hydrochloride, histidine hydrochloride or in non-salified form, for example histidine or arginine.

In one embodiment, the total molar concentration in ionic species in the composition is greater than or equal to 10 mM.

In one embodiment, the total molar concentration in ionic species in the composition is greater than or equal to 20 mM.

In one embodiment, the total molar concentration in ionic species in the composition is greater than or equal to 30 mM.

In one embodiment, the total molar concentration in ionic species in the composition is greater than or equal to 50 mM.

In one embodiment, the total molar concentration in ionic species in the composition is greater than or equal to 75 mM.

In one embodiment, the total molar concentration in ionic species in the composition is greater than or equal to 100 mM.

In one embodiment, the total molar concentration in ionic species in the composition is greater than or equal to 200 mM.

In one embodiment, the total molar concentration in ionic species in the composition is greater than or equal to 300 mM.

In one embodiment, the total molar concentration in ionic species in the composition is greater than or equal to 500 mM.

In one embodiment, the total molar concentration in ionic species in the composition is greater than or equal to 600 mM.

In one embodiment, the total molar concentration in ionic species in the composition is greater than or equal to 700 mM.

In one embodiment, the total molar concentration in ionic species in the composition is greater than or equal to 800 mM.

In one embodiment, the total molar concentration in ionic species in the composition is greater than or equal to 900 mM.

In one embodiment, the total molar concentration in ionic species in the composition is lower than or equal to 1000 mM.

In one embodiment, the total molar concentration in ionic species in the composition is lower than or equal to 1500 mM.

In one embodiment, the total molar concentration in ionic species in the composition is lower than or equal to 1200 mM.

In one embodiment, the total molar concentration in ionic species in the composition is lower than or equal to 1000 mM.

In one embodiment, the total molar concentration in ionic species in the composition is lower than or equal to 900 mM.

In one embodiment, the total molar concentration in ionic species in the composition is lower than or equal to 800 mM.

In one embodiment, the total molar concentration in ionic species in the composition is lower than or equal to 700 mM.

In one embodiment, the total molar concentration in ionic species in the composition is lower than or equal to 600 mM.

In one embodiment, the total molar concentration in ionic species in the composition is lower than or equal to 500 mM.

In one embodiment, the total molar concentration in ionic species in the composition is lower than or equal to 400 mM.

In one embodiment, the total molar concentration in ionic species in the composition is lower than or equal to 300 mM.

In one embodiment, the total molar concentration in ionic species in the composition is lower than or equal to 200 mM.

In one embodiment, the total molar concentration in ionic species in the composition is lower than or equal to 100 mM.

In one embodiment, the total molar concentration in ionic species in the composition is comprised from 10 to 1000 mM.

In one embodiment, the total molar concentration in ionic species in the composition is comprised from 20 to 1000 mM.

In one embodiment, the total molar concentration in ionic species in the composition is comprised from 30 to 1000 mM.

In one embodiment, the total molar concentration in ionic species in the composition is comprised from 50 to 1000 mM.

In one embodiment, the total molar concentration in ionic species in the composition is comprised from 75 to 1000 mM.

In one embodiment, the total molar concentration in ionic species in the composition is comprised from 100 to 1000 mM.

In one embodiment, the total molar concentration in ionic species in the composition is comprised from 200 to 1000 mM.

In one embodiment, the total molar concentration in ionic species in the composition is comprised from 300 to 1000 mM.

In one embodiment, the total molar concentration in ionic species in the composition is comprised from 400 to 1000 mM.

In one embodiment, the total molar concentration in ionic species in the composition is comprised from 500 to 1000 mM.

In one embodiment, the total molar concentration in ionic species in the composition is comprised from 600 to 1000 mM.

In one embodiment, the total molar concentration in ionic species in the composition is comprised from 10 to 900 mM.

In one embodiment, the total molar concentration in ionic species in the composition is comprised from 20 to 900 mM.

In one embodiment, the total molar concentration in ionic species in the composition is comprised from 30 to 900 mM.

In one embodiment, the total molar concentration in ionic species in the composition is comprised from 50 to 900 mM.

In one embodiment, the total molar concentration in ionic species in the composition is comprised from 75 to 900 mM.

In one embodiment, the total molar concentration in ionic species in the composition is comprised from 100 to 900 mM.

In one embodiment, the total molar concentration in ionic species in the composition is comprised from 200 to 900 mM.

In one embodiment, the total molar concentration in ionic species in the composition is comprised from 300 to 900 mM.

In one embodiment, the total molar concentration in ionic species in the composition is comprised from 400 to 900 mM.

In one embodiment, the total molar concentration in ionic species in the composition is comprised from 500 to 900 mM.

In one embodiment, the total molar concentration in ionic species in the composition is comprised from 600 to 900 mM.

In one embodiment, the total molar concentration in ionic species in the composition is comprised from 10 to 900 mM.

In one embodiment, the total molar concentration in ionic species in the composition is comprised from 20 to 800 mM.

In one embodiment, the total molar concentration in ionic species in the composition is comprised from 30 to 800 mM.

In one embodiment, the total molar concentration in ionic species in the composition is comprised from 50 to 800 mM.

In one embodiment, the total molar concentration in ionic species in the composition is comprised from 75 to 800 mM.

In one embodiment, the total molar concentration in ionic species in the composition is comprised from 100 to 800 mM.

In one embodiment, the total molar concentration in ionic species in the composition is comprised from 200 to 800 mM.

In one embodiment, the total molar concentration in ionic species in the composition is comprised from 300 to 800 mM.

In one embodiment, the total molar concentration in ionic species in the composition is comprised from 400 to 800 mM.

In one embodiment, the total molar concentration in ionic species in the composition is comprised from 500 to 800 mM.

In one embodiment, the total molar concentration in ionic species in the composition is comprised from 600 to 800 mM.

In one embodiment, the total molar concentration in ionic species in the composition is comprised from 10 to 700 mM.

In one embodiment, the total molar concentration in ionic species in the composition is comprised from 20 to 700 mM.

In one embodiment, the total molar concentration in ionic species in the composition is comprised from 30 to 700 mM.

In one embodiment, the total molar concentration in ionic species in the composition is comprised from 50 to 700 mM.

In one embodiment, the total molar concentration in ionic species in the composition is comprised from 75 to 700 mM.

In one embodiment, the total molar concentration in ionic species in the composition is comprised from 100 to 700 mM.

In one embodiment, the total molar concentration in ionic species in the composition is comprised from 200 to 700 mM.

In one embodiment, the total molar concentration in ionic species in the composition is comprised from 300 to 700 mM.

In one embodiment, the total molar concentration in ionic species in the composition is comprised from 400 to 700 mM.

In one embodiment, the total molar concentration in ionic species in the composition is comprised from 500 to 700 mM.

In one embodiment, the total molar concentration in ionic species in the composition is comprised from 600 to 700 mM.

In one embodiment, the total molar concentration in ionic species in the composition is comprised from 10 to 600 mM.

In one embodiment, the total molar concentration in ionic species in the composition is comprised from 20 to 600 mM.

In one embodiment, the total molar concentration in ionic species in the composition is comprised from 30 to 600 mM.

In one embodiment, the total molar concentration in ionic species in the composition is comprised from 50 to 600 mM.

In one embodiment, the total molar concentration in ionic species in the composition is comprised from 75 to 600 mM.

In one embodiment, the total molar concentration in ionic species in the composition is comprised from 100 to 600 mM.

In one embodiment, the total molar concentration in ionic species in the composition is comprised from 200 to 600 mM.

In one embodiment, the total molar concentration in ionic species in the composition is comprised from 300 to 600 mM.

In one embodiment, the total molar concentration in ionic species in the composition is comprised from 400 to 600 mM.

In one embodiment, the total molar concentration in ionic species in the composition is comprised from 500 to 600 mM.

In one embodiment, the total molar concentration in ionic species in the composition is comprised from 10 to 500 mM.

In one embodiment, the total molar concentration in ionic species in the composition is comprised from 20 to 500 mM.

In one embodiment, the total molar concentration in ionic species in the composition is comprised from 30 to 500 mM.

In one embodiment, the total molar concentration in ionic species in the composition is comprised from 50 to 500 mM.

In one embodiment, the total molar concentration in ionic species in the composition is comprised from 75 to 500 mM.

In one embodiment, the total molar concentration in ionic species in the composition is comprised from 100 to 500 mM.

In one embodiment, the total molar concentration in ionic species in the composition is comprised from 200 to 500 mM.

In one embodiment, the total molar concentration in ionic species in the composition is comprised from 300 to 500 mM.

In one embodiment, the total molar concentration in ionic species in the composition is comprised from 400 to 500 mM.

In one embodiment, the total molar concentration in ionic species in the composition is comprised from 10 to 400 mM.

In one embodiment, the total molar concentration in ionic species in the composition is comprised from 20 to 400 mM.

In one embodiment, the total molar concentration in ionic species in the composition is comprised from 30 to 400 mM.

In one embodiment, the total molar concentration in ionic species in the composition is comprised from 50 to 400 mM.

In one embodiment, the total molar concentration in ionic species in the composition is comprised from 75 to 400 mM.

In one embodiment, the total molar concentration in ionic species in the composition is comprised from 100 to 400 mM.

In one embodiment, the total molar concentration in ionic species in the composition is comprised from 200 to 400 mM.

In one embodiment, the total molar concentration in ionic species in the composition is comprised from 300 to 400 mM.

In one embodiment, the total molar concentration in ionic species in the composition is comprised from 10 to 300 mM.

In one embodiment, the total molar concentration in ionic species in the composition is comprised from 20 to 300 mM.

In one embodiment, the total molar concentration in ionic species in the composition is comprised from 30 to 300 mM.

In one embodiment, the total molar concentration in ionic species in the composition is comprised from 50 to 300 mM.

In one embodiment, the total molar concentration in ionic species in the composition is comprised from 75 to 300 mM.

In one embodiment, the total molar concentration in ionic species in the composition is comprised from 100 to 300 mM.

In one embodiment, the total molar concentration in ionic species in the composition is comprised from 200 to 300 mM.

In one embodiment, the total molar concentration in ionic species in the composition is comprised from 10 to 200 mM.

In one embodiment, the total molar concentration in ionic species in the composition is comprised from 20 to 200 mM.

In one embodiment, the total molar concentration in ionic species in the composition is comprised from 30 to 200 mM.

In one embodiment, the total molar concentration in ionic species in the composition is comprised from 50 to 200 mM.

In one embodiment, the total molar concentration in ionic species in the composition is comprised from 75 to 200 mM.

In one embodiment, the total molar concentration in ionic species in the composition is comprised from 100 to 200 mM.

In one embodiment, the total molar concentration in ionic species in the composition is comprised from 10 to 100 mM.

In one embodiment, the total molar concentration in ionic species in the composition is comprised from 20 to 100 mM.

In one embodiment, the total molar concentration in ionic species in the composition is comprised from 30 to 100 mM.

In one embodiment, the total molar concentration in ionic species in the composition is comprised from 50 to 100 mM.

In one embodiment, the total molar concentration in ionic species in the composition is comprised from 75 to 100 mM.

In one embodiment, the total molar concentration in ionic species in the composition is comprised from 10 to 75 mM.

In one embodiment, the total molar concentration in ionic species in the composition is comprised from 20 to 75 mM.

In one embodiment, the total molar concentration in ionic species in the composition is comprised from 30 to 75 mM.

In one embodiment, the total molar concentration in ionic species in the composition is comprised from 50 to 75 mM.

In one embodiment, the total molar concentration in ionic species in the composition is comprised from 10 to 50 mM.

In one embodiment, the total molar concentration in ionic species in the composition is comprised from 20 to 50 mM.

In one embodiment, the total molar concentration in ionic species in the composition is comprised from 30 to 50 mM.

In one embodiment, said ionic species are present in a concentration ranging from 5 to 400 mM.

In one embodiment, said ionic species are present in a concentration ranging from 5 to 300 mM.

In one embodiment, said ionic species are present in a concentration ranging from 5 to 200 mM.

In one embodiment, said ionic species are present in a concentration ranging from 5 to 100 mM.

In one embodiment, said ionic species are present in a concentration ranging from 5 to 75 mM.

In one embodiment, said ionic species are present in a concentration ranging from 5 to 50 mM.

In one embodiment, said ionic species are present in a concentration ranging from 5 to 25 mM.

In one embodiment, said ionic species are present in a concentration ranging from 5 to 20 mM.

In one embodiment, said ionic species are present in a concentration ranging from 5 to 10 mM.

In one embodiment, said ionic species are present in a concentration ranging from 10 to 400 mM.

In one embodiment, said ionic species are present in a concentration ranging from 10 to 300 mM.

In one embodiment, said ionic species are present in a concentration ranging from 10 to 200 mM.

In one embodiment, said ionic species are present in a concentration ranging from 10 to 100 mM.

In one embodiment, said ionic species are present in a concentration ranging from 10 to 75 mM.

In one embodiment, said ionic species are present in a concentration ranging from 10 to 50 mM.

In one embodiment, said ionic species are present in a concentration ranging from 10 to 25 mM.

In one embodiment, said ionic species are present in a concentration ranging from 10 to 20 mM.

In one embodiment, said ionic species are present in a concentration ranging from 20 to 300 mM.

In one embodiment, said ionic species are present in a concentration ranging from 20 to 200 mM.

In one embodiment, said ionic species are present in a concentration ranging from 20 to 100 mM.

In one embodiment, said ionic species are present in a concentration ranging from 20 to 75 mM.

In one embodiment, said ionic species are present in a concentration ranging from 20 to 50 mM.

In one embodiment, said ionic species are present in a concentration ranging from 20 to 25 mM.

In one embodiment, said ionic species are present in a concentration ranging from 50 to 300 mM.

In one embodiment, said ionic species are present in a concentration ranging from 50 to 200 mM.

In one embodiment, said ionic species are present in a concentration ranging from 50 to 100 mM.

In one embodiment, said ionic species are present in a concentration ranging from 50 to 75 mM.

In one embodiment, the compositions according to the invention further comprise buffers.

In one embodiment, compositions according to the invention comprise buffers at a concentration from 0 to 100 mM.

In one embodiment, compositions according to the invention comprise buffers at a concentration from 15 to 50 mM.

In one embodiment, the compositions according to the invention comprise a buffer chosen in the group consisting of a phosphate buffer, Tris (tris hydroxymethyl aminomethane) and sodium citrate.

In one embodiment, the buffer is sodium phosphate.

In one embodiment, the buffer is Tris (tris hydroxymethyl aminomethane).

In one embodiment, the buffer is sodium citrate.

In one embodiment, compositions according to the invention further comprise zinc salts at a concentration from 0 to 5000 μM.

In one embodiment, compositions according to the invention further comprise zinc salts at a concentration from 0 to 4000 μM.

In one embodiment, compositions according to the invention further comprise zinc salts at a concentration from 0 to 3000 μM.

In one embodiment, compositions according to the invention further comprise zinc salts at a concentration from 0 to 2000 μM.

In one embodiment, compositions according to the invention further comprise zinc salts at a concentration from 0 to 1000 μM.

In one embodiment, compositions according to the invention further comprise zinc salts at a concentration from 50 to 600 μM.

In one embodiment, compositions according to the invention further comprise zinc salts at a concentration from 100 to 500 μM.

In one embodiment, compositions according to the invention further comprise zinc salts at a concentration from 200 to 500 μM.

In one embodiment, the compositions according to the invention further comprise a surfactant.

In one embodiment, the surfactant is chosen in the group consisting of propylene glycol and polysorbate.

The compositions according to the invention may further comprise additives such as tonicity agents.

In one embodiment, the tonicity agents are chosen in the group consisting of glycerin, sodium chloride, mannitol and glycine.

The compositions according to the invention may further comprise all excipients compatible with pharmacopoeia and compatible with insulins used at the usage concentrations.

The invention also relates to a pharmaceutical formulation according to the invention, characterized in that it is obtained by drying and/or freeze drying.

In the case of local and systemic releases, the routes of administration envisaged are intravenous, subcutaneous, intradermal or intramuscular.

Transdermal, oral, nasal, vaginal, ocular, oral, and pulmonary routes of administration are also considered.

In one embodiment, the composition according to the invention is characterized in that it is administered once a day.

In one embodiment, the composition according to the invention is characterized in that it is administered 2 times a day.

In one embodiment, the composition according to the invention is characterized in that it is administered 2 times a day.

In one embodiment, the composition according to the invention is characterized in that it further comprises a prandial insulin.

In one embodiment, the composition according to the invention further comprises at least one prandial insulin and is characterized in that it is administered once a day.

In one embodiment, the composition according to the invention further comprises at least one prandial insulin and is characterized in that it is administered 2 times a day.

In one embodiment, the composition according to the invention further comprises at least one prandial insulin and is characterized in that it is administered 2 times a day.

In one embodiment, the composition according to the invention is characterized in that it further comprises a gut hormone.

In one embodiment, the composition according to the invention further comprises at least one gut hormone is characterized in that it is administered once a day.

In one embodiment, the composition according to the invention further comprises at least one gut hormone and is characterized in that it is administered 2 times a day.

In one embodiment, the composition according to the invention further comprises at least one gut hormone is characterized in that it is administered 2 times a day.

In one embodiment, the composition according to the invention is characterized in that the gut hormone is a GLP-1 RA.

In one embodiment, the composition according to the invention further comprises at least one GLP-1 RA is characterized in that it is administered once a day.

In one embodiment, the composition according to the invention further comprises at least one GLP-1 RA is characterized in that it is administered 2 times a day.

In one embodiment, the composition according to the invention further comprises at least one GLP-1 RA is characterized in that it is administered 2 times a day.

The invention also relates to single-dose formulations at pH from 6.0 to 8.0 comprising a basal insulin whose isoelectric point is from 5.8 to 8.5.

The invention also relates to single-dose formulations at pH from 6.0 to 8.0 comprising a basal insulin whose isoelectric point is from 5.8 to 8.5 and a prandial insulin.

The invention also relates to single-dose formulations at pH from 6.0 to 8.0 comprising a basal insulin whose isoelectric point is from 5.8 to 8.5 and a gut hormone, as defined above.

The invention also relates to single-dose formulations with at pH from 6.0 to 8.0 comprising a basal insulin whose isoelectric point is from 5.8 to 8.5, a prandial insulin and a gut hormone, as defined above.

The invention also relates to single-dose formulations with at pH from 6.6 to 7.8 comprising a basal insulin whose isoelectric point is from 5.8 to 8.5.

The invention also relates to single-dose formulations at pH from pH of from 6.6 to 7.8 comprising a basal insulin whose isoelectric point is from 5.8 to 8.5 and a prandial insulin.

The invention also relates to single-dose formulations at pH from pH of from 6.6 to 7.8 comprising a basal insulin whose isoelectric point is from is from 5.8 to 8.5 and a gut hormone, as defined above.

The invention also relates to single-dose formulations at pH from pH of from 6.6 to 7.8 comprising a basal insulin whose isoelectric point is from 5.8 to 8.5, a prandial insulin and a gut hormone, as defined above.

The invention also relates to single-dose formulations at pH from 6.6 to 7.6 comprising a basal insulin whose isoelectric point is from 5.8 to 8.5.

The invention also relates to single-dose formulations at pH from 6.6 to 7.6 comprising a basal insulin whose isoelectric point is from 5.8 to 8.5 and a prandial insulin.

The invention also relates to single-dose formulations at pH from 6.6 to 7.6 comprising a basal insulin whose isoelectric point is from 5.8 to 8.5 and a gut hormone, as defined above.

The invention also relates to single-dose formulations at pH from 6.6 to 7.6 comprising a basal insulin whose isoelectric point is from 5.8 to 8.5, a prandial insulin and a gut hormone, as defined above.

In one embodiment, the single-dose formulations further comprise a co-polyamino acid as defined above.

In one embodiment, the formulations are in the form of an injectable solution.

In one embodiment, the basal insulin whose isoelectric point is from 5.8 to 8.5 is insulin glargine

In one embodiment, the GLP-1 RA, analogue or derivative of GLP-1 RA is chosen in the group comprising exenatide (Byetta®), liraglutide (Victoza®), lixisenatide (Lyxumia®), albiglutide (Tanzeum®), dulaglutide (Trulicity®) or one of their derivatives.

In one embodiment, the gut hormone is exenatide.

In one embodiment, the gut hormone is liraglutide.

In one embodiment, the gut hormone is lixisenatide.

In one embodiment, the gut hormone is albiglutide.

In one embodiment, the gut hormone is dulaglutide.

The solubilization at a pH from 6.0 to 8.0 of the basal insulins whose isoelectric point is from 5.8 to 8.5, by the co-polyamino acids bearing carboxylate charges and at least one hydrophobic radical according to the invention can be observed and controlled in a simple manner, with the naked eye, by means of a change in the appearance of the solution.

The solubilization at a pH from 6.6 to 7.8 of the basal insulins whose isoelectric point is from 5.8 to 8.5, by the co-polyamino acids bearing carboxylate charges and at least one hydrophobic radical according to the invention can be observed and controlled in a simple manner, with the naked eye, by means of a change in the appearance of the solution.

Moreover, and just as importantly, the Applicant has been able to verify that a basal insulin whose isoelectric point is from 5.8 to 8.5, solubilized at a pH from 6.0 to 8.0 in the presence of a co-polyamino acid bearing carboxylate charges and at least one hydrophobic radical according to the invention preserves its slow-acting insulin action whether alone or in combination with a prandial insulin or a gut hormone.

The applicant has also been able to verify that a prandial insulin mixed at pH from 6.0 to 8.0 in the presence of a co-polyamino acid bearing carboxylate charges and at least one hydrophobic radical according to the invention and of a basal insulin whose isoelectric point is from 5.8 to 8.5, preserves its fast-acting insulin action.

The preparation of a composition according to the invention has the advantage of being able to be performed by simple mixing of an aqueous solution of basal insulin whose isoelectric point is from 5.8 to 8.5, and of a co-polyamino acid bearing carboxylate charges and at least one hydrophobic radical according to the invention, in an aqueous solution or in freeze-dried form. If necessary, the pH of the preparation is adjusted to a pH from 6.0 to 8.0.

The preparation of a composition according to the invention has the advantage of being able to be performed by simple mixing of an aqueous solution of basal insulin whose isoelectric point is from 5.8 to 8.5, and of a co-polyamino acid bearing carboxylate charges and at least one hydrophobic radical according to the invention, in an aqueous solution or in freeze-dried form. If necessary, the pH of the preparation is adjusted to a pH from 6.0 to 8.0.

The preparation of a composition according to the invention has the advantage of being able to be performed by simple mixing of an aqueous solution of basal insulin whose isoelectric point is from 5.8 to 8.5, of a solution of GLP-1 RA, an analogue or a derivative of GLP-1 RA, and a co-polyamino acid bearing carboxylate charges and at least one hydrophobic radical according to the invention, in an aqueous solution or in freeze-dried form. If necessary, the pH of the preparation is adjusted to a pH from 6.0 to 8.0.

The preparation of a composition according to the invention has the advantage of being able to be performed by simple mixing of an aqueous solution of basal insulin whose isoelectric point is from 5.8 to 8.5, of a solution of prandial insulin, of a solution of GLP-1 RA, an analogue or a derivative of GLP-1 RA, and a co-polyamino acid bearing carboxylate charges and at least one hydrophobic radical according to the invention, in an aqueous solution or in freeze-dried form. If necessary, the pH of the preparation is adjusted to a pH from 6.0 to 8.0.

In one embodiment, the mixture of the basal insulin and the co-polyamino acid is concentrated via ultrafiltration before the mixture with the prandial insulin in an aqueous solution or in freeze-dried form.

If necessary, the composition of the mixture is adjusted with excipients such as glycerin, zinc chloride, and polysorbate (Tween®) by adding concentrated solutions of these excipients into the mixture. If necessary, the pH of the preparation is adjusted to a pH from 6.0 to 8,

### EXAMPLES

### Part A—Synthesis of Hydrophobic Intermediate Compounds Hyd to Obtain the Radicals -Hy

TABLE 1
List and structure of hydrophobic molecules precursors of hydrophobic radicals
before grafting on the co-polyamino acid.

| No. | HYDROPHOBIC INTERMEDIATE COMPOUNDS |
|---|---|
| A1 | 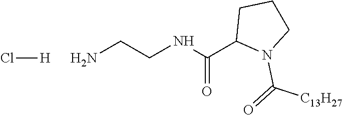 |
| A2 | 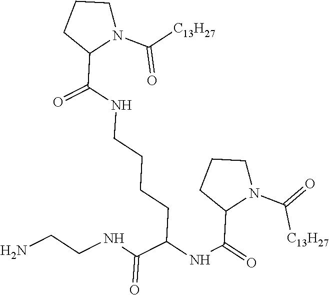 |
| A3 | 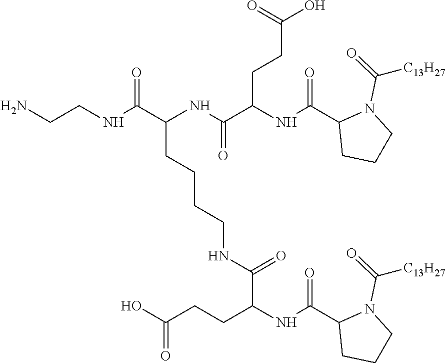 |
| A4 | 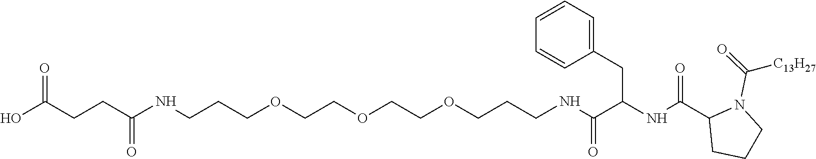 |
| A5 | 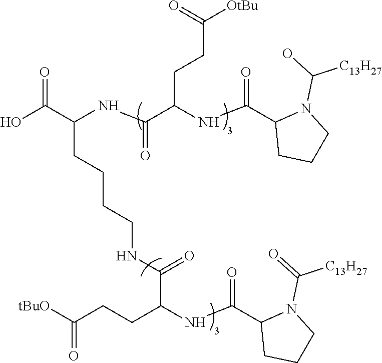 |
| A6 | 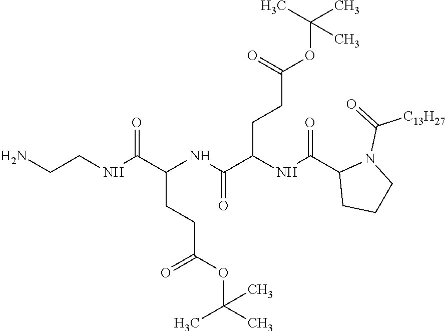 |

### Example A1: Molecule A1

### Molecule 1: Product Obtained by the Reaction Between Myristoyl Chloride and L-Proline.

To a solution of L-proline (300.40 g, 2.61 mol) in 2N aqueous sodium hydroxide solution (1.63 L) at 0° C. is slowly added over 1 h myristoyl chloride (322 g, 1.30 mol) in solution in dichloromethane (DCM, 1.63 L). At the end of the addition, the reaction medium is raised to 20° C. in 3 h, then stirred for 2 more hours. The mixture is cooled to 0° C. and then a 37% HCl aqueous solution (215 m1) is added in 15 minutes. The reaction medium is stirred for 1 h from 0° C. to 20° C. The organic phase is separated, washed with a 10% HCl aqueous solution (3×430 mL), a saturated NaCl aqueous solution (430 mL), dried over Na₂SO₄, filtered through cotton and then concentrated under reduced pressure. The residue is solubilized in heptane (1.31 L) at 50° C., then the solution is progressively returned to room temperature. After priming the crystallization with a glass rod, the medium is again heated at 40° C. for 30 minutes and then returned to room temperature for 4 h. A white solid is obtained after filtration on a sintered filter, washing with heptane (2×350 mL) and drying under reduced pressure.

Yield: 410 g (97%)

¹H NMR (CDCl₃, ppm): 0.88 (3H); 1.28 (20H); 1.70 (2H); 1.90-2.10 (3H); 2.36 (2H); 2.51 (1H); 3.47 (1H); 3.56 (1H); 4.61 (1H).

LC/MS (ESI): 326.4; 651.7; (calculated ([M+H]⁺): 326.3; ([M+H]⁺): 651.6).

### Molecule 2: Product Obtained by the Reaction Between Molecule 1 and N-Boc Ethylenediamine.

To a solution of molecule 1 (190.0 g, 583.7 mmol) at 0° C. in DCM (2.9 L) is added 1-hydroxybenzotriazole (HOBt, 8.94 g, 58.37 g). mmol), then N-Boc-ethylenediamine (BocEDA, 112.2 g, 700.5 mmol) dissolved in DCM (150 mL) is introduced over a period of 15 min. (3-Dimethylaminopropyl)-N′-ethyl carbodiimide hydrochloride (EDC, 123.1 g, 642.1 mmol) is then added portion-wise and the mixture is stirred for 1 h at 0° C. and 17 h from 0° C. and room temperature. The reaction mixture is then washed with a saturated NaHCO₃ aqueous solution (2×1.5 L), an aqueous solution of 1N HCl (2×1.5 L), a saturated NaCl aqueous solution (1.5 L), then dried over Na₂SO₄, filtered and concentrated under reduced pressure. A white solid is obtained after crystallization in acetonitrile.

Yield: 256.5 g (93%).

¹H NMR (CDCl₃, ppm): 0.88 (3H); 1.16-1.38 (20H); 1.44 (9H); 1.56-1.71 (2H); 1.78-2.45 (6H); 3.11-3.72 (6H); 4.30 (0.1H); 4.51 (0.9H); 4.87 (0.1H); 5.04 (0.9H); 6.87 (0.1H); 7.23 (0.9H).

LC/MS (ESI): 468.3; (calculated ([M+H]⁺): 468.4).

### Molecule A1

To a solution of molecule 2 (256.5 g, 548.4 mmol) in DCM (2.75 L) is added drop by drop and at 0° C. a solution of 4 M hydrochloric acid in dioxane (685 mL, 2.74 mol). After stirring for 16 h at 0° C., the reaction medium is brought to ambient temperature over 1 h and the solution is concentrated under reduced pressure. The residue is triturated in pentane (1.6 L) sinter filtered and dried at 40° C. under reduced pressure to give a white solid of A1 molecule in the form of hydrochloride salt.

Yield: 220.0 g (99%)

¹H NMR (MeOD-d4, ppm): 0.90 (3H); 1.21-1.43 (20H); 1.54-1.66 (2H); 1.85-2.28 (4H); 2.39 (2H); 3.00-3.17 (2H); 3.30-3.40 (1H); 3.43-3.71 (3H); 4.29 (0.94H); 4.48 (0.06H).

LC/MS (ESI): 368.2; (calculated ([M+H]⁺): 368.3).

### Example A2: Molecule A2

### Molecule 3: Product Obtained by the Reaction Between Molecule 1 and L-Lysine

To a solution of the molecule 1 (356.1 g, 1.1 mol) in tetrahydrofuran (THF, 1.7 L) at 0° C. are successively added N-hydroxy succinimide (NHS, 132.2 g, 1.15 mol) followed by N,N′-dicyclohexylcarbodiimide (DCC, 237.1 g, 1.15 mol). The reaction medium is stirred for 43 hours between 0° C. and room temperature, sinter filtered, and then added over 50 min to a solution of L-lysine (84 g, 574.5 mmol) and N,N-diisopropylethylamine (DPEIA, 707.1 g, 5.47 mol) in water (220 mL). After stirring for 17 hours at room temperature, the medium is concentrated under reduced pressure, the residue is diluted with water (3 L) and the aqueous phase is washed with ethyl acetate (EtOAc), 2×1.3 L) then acidified to pH 1 by the addition of a 6N HCl aqueous solution. The aqueous phase is extracted with DCM, the organic phase is then washed with a saturated NaCl aqueous solution (2×1.3 L), dried over Na₂SO₄, filtered on cotton and concentrated under reduced pressure. A white solid of molecule 3 is obtained after crystallization in acetone.

Yield: 224.2 g (54%)

¹H NMR (DMSO-d₆, ppm): 0.85 (6H); 1.06-2.30 (62H); 2.90-3.10 (2H); 3.25-3.59 (4H); 4.06-4.30 (2H); 4.30-4.42 (1H); 7.64-7.73 (0.6H); 7.93-8.07 (1H); 8.22-8.31 (0.4H); 12.50 (1H).

LC/MS (ESI): 761.8; (calculated ([M+H]⁺): 761.6).

### Molecule 4: Product Obtained by the Coupling Between Molecule 3 and N-Boc Ethylenediamine.

By a process similar to that used for the preparation of molecule 2 applied to molecule 3 (174.0 g, 228.6 mmol) and to Boc EDA (44 g, 274.3 mmol), a white solid molecule 4 is obtained after recrystallization in acetonitrile.

Yield: 195.0 g (94%)

¹H NMR (DMSO-d₆, ppm): 0.85 (6H); 1.10-2.30 (71H); 2.95-3.10 (6H); 3.31-3.55 (4H); 4.10-4.40 (3H); 6.35-6.75 (1H); 7.60-8.20 (3H).

LC/MS (ESI): 903.7; (calculated ([M+H]⁺): 903.7).

### Molecule A2

After a process similar to that used for the preparation of molecule A1 and applied to molecule 4 (192.3 g, 212.9 mmol) the residue obtained after evaporation of the reaction mixture under reduced pressure is diluted in DCM (1.1 L), the organic phase is washed with a 2M sodium hydroxide aqueous solution (2×0.7 L), dried over Na₂SO₄, filtered through cotton and concentrated under reduced pressure. A white solid of molecule A2 is obtained after crystallization in acetonitrile.

Yield: 152.1 g (89%)

¹H NMR (DMSO-d₆, ppm): 0.85 (6H); 1.10-2.35 (64H); 2.55 (2H); 3.02 (2H); 3.25-3.65 (6H); 4.05-4.45 (3H); 7.50-8.20 (3H).

LC/MS (ESI): 803.9; (calculated ([M+H]⁺): 803.7).

### Example A3: Molecule A3

Molecule A3 is obtained by the conventional method of solid phase peptide synthesis (SPPS) on 2-chlorotrityl chloride resin (CTC) (40.00 g, 1.16 mmol/g).

Grafting of ethylene diamine (20.0 equivalents) is carried out in DCM (10 V). The unreacted sites are capped with methanol (0.8 mL/g resin) at the end of the reaction.

Couplings of Fmoc-Lys(Fmoc)-OH protected amino acids (1.5 equivalents), Fmoc-Glu(OtBu)-OH (2.5 equivalents) and of molecule 1 (2.5 equivalents) are performed in DMF (10 V), in the presence of HATU (1.0 equivalent relative to the acid) and DPEIA (1.5 equivalents relative to the acid).

The Fmoc protecting groups are removed using an 80:20 solution of DMF/piperidine (10 V).

The product is cleaved from the resin using a 50:50 DCM/TFA solution (10 V). After evaporation, the residue is solubilized in water (600 mL), the pH of the solution is adjusted to 7 by adding a solution of 5 N NaOH, and the product is lyophilized. The lyophilizate is purified by chromatography column on silica gel (dichloromethane, methanol, NH₄OH) to give the A3 molecule in the form of a white solid.

Yield: 24.6 g (50% overall in 7 steps).

¹H NMR (MeOD-d4, ppm): 0.90 (6H); 1.18-2.45 (68H); 2.45-2.60 (2H); 3.05-3.11 (2H); 3.11-3.19 (1H); 3.23-3.33 (1H); 3.43-3.66 (4H); 3.82-3.94 (2H); 4.10-4.51 (5H).

LC/MS (ESI+): 1061.9 (calculated ([M+H]⁺): 1061.8).

### Example A4: Molecule A4

### Molecule 7: Product Obtained by Solid Phase Peptide Synthesis (SPPS)

Molecule 7 is obtained by the conventional method of solid phase peptide synthesis (SPPS) on 2-chlorotrityl chloride resin (CTC) (25.00 g, 1.24 mmol/g).

Grafting of 4,7,10-trioxa-1,13-tridecanediamine (TOTA, 20.0 equivalents) is carried out in DCM (15 V). The unreacted sites are capped with methanol (0.8 mL/g resin) at the end of the reaction.

Couplings of the protected amino acid Fmoc-Phe-OH (3.0 equivalents) and molecule 1 (3.0 equivalents) are performed in DMF (10 V), in the presence of HATU (1.0 equivalent relative to the acid) and DPEIA (2.0 equivalents relative to the acid).

The Fmoc protecting groups are removed using an 80:20 DMF/piperidine solution of (10 V).

The product is cleaved from the resin using a 50:50 DCM/TFA solution (10 V). After evaporation, the residue is solubilized in DCM (500 mL) and the organic phase is washed with an aqueous solution of carbonate buffer at pH 10.4 (3×250 mL). After drying over Na₂SO₄, the organic phase is filtered, then concentrated under reduced pressure. An orange oil is obtained from molecule 7.

Yield: 15.07 g (72%)

¹H NMR (CDCl₃, ppm): 0.87 (3H); 1.08-1.42 (20H); 1.42-1.62 (2H); 1.62-1.99 (7H); 1.99-2.26 (3H); 2.72 (2H); 2.86 (2H); 2.94-3.72 (18H); 4.20-4.72 (2H); 6.63-7.37 (7H).

LC/MS (ESI): 675.6; (calculated ([M+H]⁺): 675.5).

### Molecule A4

To a solution of molecule 7 (13.79 g, 20.43 mmol) in THF (70 mL) are successively added succinic anhydride (5.11 g, 51.06 mmol) and DPEIA (8.90 mL, 51.06 mmol). The mixture is stirred for 4 h at room temperature. Dichloromethane (140 m1) is added and the organic phase is washed with an aqueous solution of 1 N HCl (2×140 m1), dried over Na₂SO₄, filtered and concentrated under reduced pressure. The crude product is purified by flash chromatography (eluent: DCM, methanol). A colorless oil is obtained from molecule A4, contaminated with residual traces of succinic acid. This product is solubilized in DCM (160 mL) and then washed with a saturated NaCl aqueous solution (160 mL), 0.1 N HCl aqueous solution (160 mL) and a saturated NaCl aqueous solution (160 mL). The organic phase is dried over Na₂SO₄, filtered and concentrated under reduced pressure. A colorless oil is obtained from molecule A4.

Yield: 12.23 g (77%)

¹H NMR (DMSO-d₆, ppm): 0.86 (3H); 1.02-1.42 (21H); 1.42-2.20 (10H); 2.23-2.38 (3H); 2.42 (2H); 2.78-3.18 (6H); 3.23-3.59 (14H); 4.12-4.58 (2H); 7.10-7.30 (5H); 7.53-8.33 (3H); 12.08 (1H).

LC/MS (ESI): 775.5; (calculated ([M+H]⁺): 775.5).

### Example A5: Molecule A5

Molecule A5 is obtained by the conventional method of solid phase peptide synthesis (SPPS) on 2-chlorotrityl chloride resin (CTC) (8.00 g, 1.24 mmol/g).

Grafting of the first Fmoc-Lys(Fmoc)-OH amino acid (2.5 equivalents) is carried out in DCM (15 V) in the presence of DPEIA (5.0 equivalents). The unreacted sites are capped with methanol (0.8 mL/g resin) at the end of the reaction. Couplings of Fmoc-Glu(OtBu)-OH protected amino acids (5.0 equivalents (×3)) and of molecule 1 (5.0 equivalents) are performed in DMF (15 V), in the presence of HATU (1.0 equivalents relative to the acid) and DPEIA (2.0 equivalents relative to the acid).

The Fmoc protecting groups are removed using an 80:20 of DMF/piperidine solution (15 V).

The product is cleaved from the resin using an 80:20 DCM/HFIP solution (15 V).

After concentration under reduced pressure, two co-evaporations are carried out on the residue with dichloromethane and the product is then purified by chromatography on silica gel (dichloromethane, methanol). A white solid is obtained from molecule A5.

Yield: 9.2 g (50% over 10 steps)

¹H NMR (CD₃OD, ppm): 0.90 (6H); 1.22-2.53 (140H); 3.12-3.25 (2H); 3.43-3.80 (4H); 4.17-4.54 (9H).

LC/MS (ESI+): 1894.5 (calculated ([M+Na]⁺): 1894.2).

### Example A6: Molecule A6

### Molecule 8: Product Obtained by SPPS

Molecule 8 is obtained by the conventional method of solid phase peptide synthesis (SPPS) on 2-chlorotrityl chloride resin (CTC) (50.0 g, 1.14 mmol/g).

Grafting of the first amino acid Fmoc-Glu (OtBu)-OH (1.3 equivalents) is carried out in DCM (10V) in the presence of DPEIA (2.6 equivalents). The unreacted sites are capped with methanol (0.8 mL/g resin) at the end of the reaction.

Couplings of the Fmoc-Glu(OtBu)-OH protected amino acid (1.3 equivalents) and of molecule 1 (3.0 equivalents) are performed in DMF (10V), in the presence of HATU (1.0 equivalent relative to the acid) and DPEIA (1.5 equivalents relative to the acid).

The Fmoc protecting groups are removed using an 80:20 DMF/piperidine solution (10 V).

The product is cleaved from the resin using an 80:20 DCM/HFIP solution (10 V).

After concentration under reduced pressure, the residue is purified by trituration in diisopropyl ether.

Yield: 35.78 g (90%)

¹H NMR (CDCl₃, ppm): 0.88 (3H); 1.19-1.35 (20H); 1.43 (9H); 1.44 (9H); 1.55-1.67 (2H); 1.90-2.46 (14H); 3.46-3.54 (1H); 3.63-3.71 (1H); 4.33-4.40 (1H); 4.43-4.52 (2H); 7.35 (0.05H); 7.40 (0.05H); 7.63 (0.95H); 7.94 (0.95H).

LC/MS (ESI+): 696.4 (calculated ([M+H]⁺): 696.5).

### Molecule 9: Product Obtained by the Reaction Between Molecule 8 and N-CBz Ethylenediamine.

By a process similar to that used for the preparation of molecule 2 and applied to molecule 8 (30.0 g, 43.11 mmol) and N-CBz ethylenediamine hydrochloride (CBzEDA.HCl, 11.93 g, 51.73 mmol), in the presence of DIPEA (15.0 mL, 86.22 mmol) and using methyl tetrahydrofuran (Me-THF) as the solvent, a beige solid is obtained from molecule 9. It is used without further purification.

Yield: 37.6 g (100%)

¹H NMR (CDCl₃, ppm): 0.88 (3H); 1.19-1.34 (20H); 1.42 (9H); 1.44 (9H); 1.52-2.54 (16H); 3.16-3.70 (6H); 4.08-4.15 (1H); 4.19-4.25 (1H); 4.43-4.53 (1H); 5.00 (1H); 5.08 (1H); 6.56 (1H); 7.00 (1H); 7.24-7.37 (5H); 7.59 (1H); 8.41 (1H).

LC/MS (ESI+): 872.5 (calculated ([M+H]⁺): 872.6).

### Molecule A6

To a solution of molecule 9 (37.6 g, 43.11 mmol) in methanol (376 mL) is added Pd/Al₂O₃ (3.76 g) under an argon atmosphere. The mixture is placed in a hydrogen atmosphere (7 bar) and stirred at room temperature for 72 h. After P4 filtration of the catalyst on sintered P4 and then on an Omnipore 0.2 μm PTFE hydrophilic membrane, the filtrate is evaporated under reduced pressure to produce the molecule A6 as a sticky oil.

Yield: 31.06 g (98%)

¹H NMR (CDCl₃, ppm): 0.88 (3H); 1.19-1.35 (20H); 1.43 (9H); 1.46 (9H); 1.56-1.67 (2H); 1.92-2.12 (6H); 2.24-2.54 (8H); 2.71 (2H); 2.90 (2H); 3.22-3.32 (1H); 3.42-3.51 (1H); 3.55-3.64 (1H); 3.73-3.81 (1H); 4.13-4.21 (1H); 4.26-4.33 (1H); 4.39-4.48 (1H); 7.10 (1H); 7.71 (1H); 8.45 (1H).

LC/MS (ESI+): 738.5 (calculated ([M+H]⁺): 738.5).

### Part B—Synthesis of Hydrophobic Co-Polyamino Acids

| | CO-POLYAMINO ACIDS BEARING CARBOXYLATE CHARGES |
|---|---|
| No. | AND HYDROPHOBIC RADICALS |
| B1 | 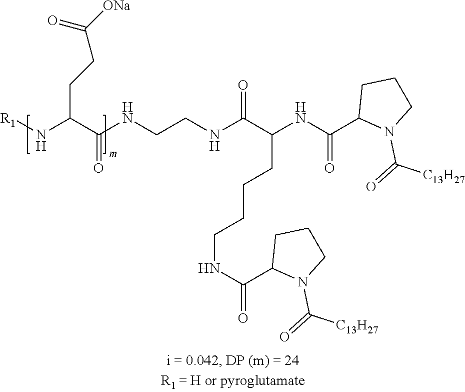 |
| B2 | 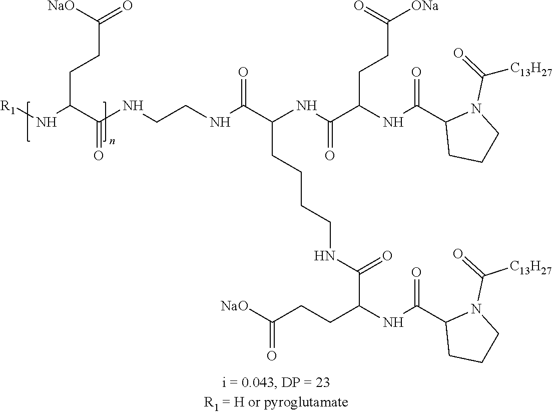 |
| B3 | 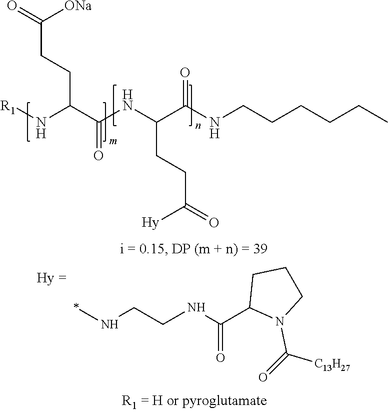 |
| B4 | 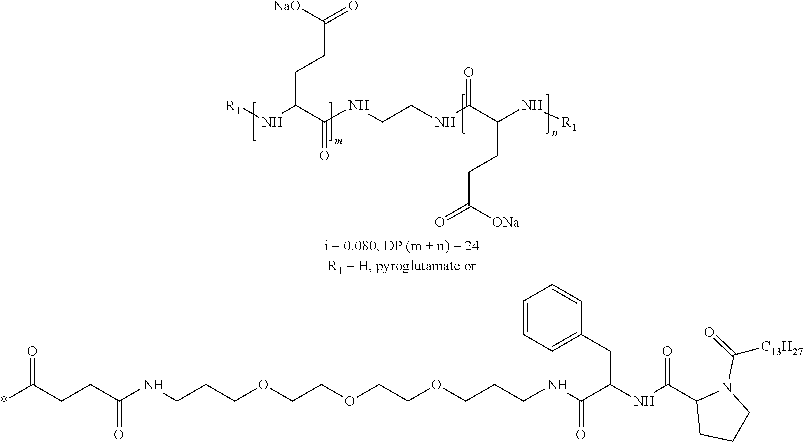 |
| B5 | 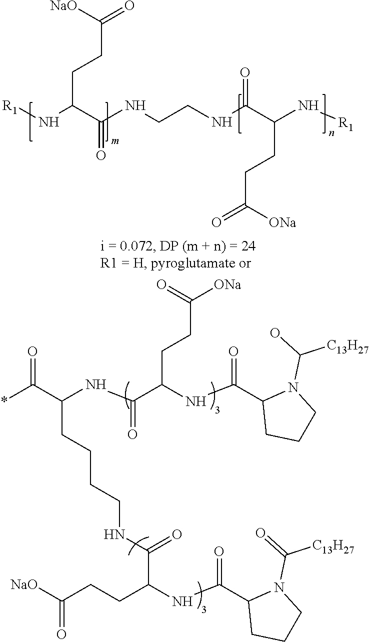 |
| B6 | 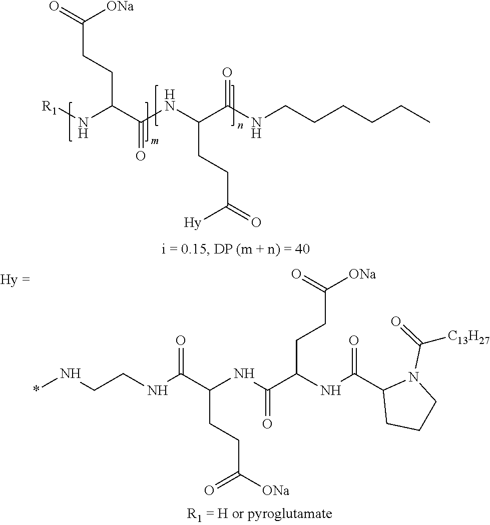 |

### Example B1

Co-Polyamino Acid B1: Sodium Poly-L-Glutamate Modified at One of its Ends by Molecule A2 and Having a Mean Number Average Molecular Mass (Mn) of 3,264 g/Mol

In a previously oven-dried flask, γ-benzyl-L-glutamate N-carboxy anhydride (144.2 g, 548 mmol) is solubilized in anhydrous DMF (525 mL). The mixture is stirred under argon until complete solubilization, cooled to −10° C., and a solution of molecule A2 (20.0 g, 24.9 mmol) in DCM (100 mL) is introduced rapidly. The mixture is stirred for 13 h at 0° C., 6 h at 20° C. and then heated at 65° C. for 2 h. All of the DCM and 60% of the DMF are distilled under reduced pressure at 70° C., then the reaction medium is cooled to 55° C. and methanol (1.1 L) is added over a period of 50 min. The suspension obtained is stirred for 18 h at 0° C. and then sinter filtered. The white solid of poly-L-(benzyl glutamate) (PBLG) modified by the molecule A2 obtained is rinsed with diisopropyl ether (PEI, 2×275 mL) and dried at 30° C. under reduced pressure.

The PBLG (25.0 g) is diluted in TFA (150 mL), and a 33% hydrobromic acid (HBr) solution in acetic acid (70 mL, 400 mmol) is then added drop by drop and at 0° C. The solution is then stirred for 2 h at room temperature and then cooled to 10° C. PEI (125 m1) and then water (125 m1) are introduced into the reaction mixture while maintaining the temperature at 10° C. The suspension obtained is stirred for 30 min, sinter filtered and rinsed with PEI (2×100 mL) followed by water (2×100 mL). The solid obtained is suspended in an aqueous solution of 0.1N NaOH (310 mL), and then solubilized by adjusting the pH to 7 by adding 1N of an aqueous sodium hydroxide solution. After complete solubilization, the pH is raised to 12 by adding 1N aqueous sodium hydroxide solution and the mixture is stirred for 30 min before being neutralized to pH 7 by adding a 27% acetic acid solution. Acetone (30% by weight) is added to the solution and the product is filtered on activated R53SLP carbon disc (3M) with a flow rate of 5.4 g/min. The acetone is then distilled at 40° C. and under reduced pressure, and the product is then purified by ultrafiltration against a solution of 0.9% NaCl and then water until the conductimetry of the permeate is lower than 50 μS/cm. The co-polyamino acid solution is then concentrated to about 30 g/L theoretical and the pH is adjusted to 7. The aqueous solution is filtered on 0.2 microns and stored at 2-8° C.

Dry extract: 25.5 mg/g
DP (estimated from ¹H NMR): 24
From ¹H NMR: i=0.042
The calculated average molar mass of co-polyamino acid B1 is 4,390 g/mol.
Aqueous HPLC-SEC (PEG Calibrator): Mn=3,264 g/mol.

### Example B2

Co-Polyamino Acid B2: Sodium Poly-L-Glutamate Modified at One of its Ends by Molecule A3 and Having a Mean Number Average Molecular Mass (Mn) of 2,100 g/Mol

In a previously oven-dried flask, γ-benzyl-L-glutamate N-carboxy anhydride (72.46 g, 275.2 mmol) is solubilized in anhydrous DMF (270 mL). The mixture is then stirred until complete solubilization, cooled to 0° C., then a solution of molecule A3 (13.28 g, 12.51 mmol) in CHCl₃ (53 mL) is introduced rapidly. The mixture is stirred at between 0° C. and room temperature for 18 h, then heated at 65° C. for 2 h. About half of the solvent is removed by distillation and then the cooled reaction mixture at room temperature is poured drop by drop into diisopropyl ether (2.4 L) with stirring. The white precipitate is recovered by filtration, washed twice with diisopropyl ether and then dried under reduced pressure at 30° C. to obtain a white solid.

The precipitate is solubilized in DMAc (300 mL) and then Pd/Al₂O₃ (6.0 g) is added under an argon atmosphere. The mixture is placed in a hydrogen atmosphere (10 bar) and stirred at 60° C. for 24 h. After cooling to room temperature and filtration of the catalyst on a sintered P4 then through an Omnipore 0.2 μm PTFE hydrophilic membrane, a solution of water at pH 2 (6 V) is poured drop by drop on the solution of DMAc over a period of 45 minutes with stirring. After 18 h, with stirring, the white precipitate is recovered by filtration, washed with water and then dried under reduced pressure. The solid obtained is then solubilized in water (2.2 L) by adjusting the pH to 7 by adding 1N aqueous sodium hydroxide solution. The pH is then adjusted to pH 12 and the solution is maintained under agitation for 1 h. After neutralization to pH 7, the solution is filtered through a 0.2 μm filter, diluted with ethanol to obtain a solution containing ethanol at 30% mass, and then filtered through an activated carbon filter (3M R53SLP). The solution obtained is filtered through a 0.45 μm filter and purified by ultrafiltration against a 0.9% NaCl solution and then water until the conductivity of the permeate is less than 50 μS/cm. The co-polyamino acid solution is then concentrated and the pH is adjusted to 7. The aqueous solution is filtered through a 0.2 μm filter and preserved at 4° C.

Dry extract: 29.9 mg/g
DP (estimate by ¹H NMR)=23
From ¹H NMR: i=0.043
The calculated average molar mass of co-polyamino acid B2 is 4,541 g/mol.
Aqueous HPLC-SEC (PEG Calibrator): Mn=2,100 g/mol.

### Example B3

Co-Polyamino Acid B3: Sodium Poly-L-Glutamate Modified by Molecule A1 and Having a Mean Number Average Molecular Mass (Mn) of 4,771 g/Mol
Co-Polyamino Acid B3-1: Poly-L-Glutamic Acid of a Number-Average Molar Mass (Mn) 5200 g/Mol from the Polymerization of γ-Benzyl-L-Glutamate N-Carboxy Anhydride Initiated by Hexylamine

In a previously oven-dried flask, γ-benzyl-L-glutamate N-carboxy anhydride (200.8 g, 763 mmol) is solubilized in anhydrous DMF (480 mL). The mixture is then stirred under argon until complete dissolution, cooled to 0° C., and then hexylamine (2.5 mmol, 19.1 mmol) is introduced rapidly. The mixture is then stirred at 0° C. and at room temperature for 18 h. The reaction medium is then heated at 70° C. for 2 h, cooled to room temperature and then poured drop by drop into diisopropyl ether (6.7 L) with stirring. The white precipitate is recovered by filtration, washed with diisopropyl ether (3×450 mL) and then dried under vacuum at 30° C. to give a poly(gamma-benzyl-L-glutamic) acid (PBLG).

To a solution of PBLG (159.3 g) in trifluoroacetic acid (TFA, 730 mL) at 4° C. is added drop by drop a 33% hydrobromic acid (HBr) solution in acetic acid (510 mL, 2.9 mol). The mixture is stirred at ambient temperature for 2 h and then poured drop by drop onto a 1:1 (v/v) mixture of diisopropyl ether and water while stirring (8.7 L). After stirring for 2 h, the heterogeneous mixture is allowed to stand overnight. The white precipitate is recovered by filtration, washed with diisopropyl ether (2×725 mL) and then with water (2×725 mL).

The solid obtained is then solubilized in water (3.2 L) by adjusting the pH to 7 by adding 10 N aqueous sodium hydroxide solution and then 1N of an aqueous sodium hydroxide solution. After solubilization, the theoretical concentration is adjusted to 20 g/L theoretical by the addition of water (1.7 L). The solution is filtered through a 0.45 μm filter and then purified by ultrafiltration against a 0.9% NaCl solution and then water until the conductivity of the permeate is less than 50 μS/cm. The co-polyamino acid solution is then concentrated to a final volume of 2.5 L.

The aqueous solution is then acidified by adding 12 N of a HCl solution (55 mL) until a pH of 2 is reached. After stirring for 16 hours, the precipitate obtained is filtered off, washed with water (2×730 m1) and then dried at 30° C. under reduced pressure.

The white solid obtained is solubilized in DMF (1.16 L) and then heated at 80° C. for 22 h. After cooling to room temperature, the mixture is poured drop by drop into the water at pH 2 containing 15% by weight of NaCl (9.3 L), under agitation and the pH is maintained at 2. After stirring for 2 h, the heterogeneous mixture is allowed to stand overnight. The white precipitate is recovered by filtration, washed with water (2×1.2 L) and then dried at 30° C. under reduced pressure to give a poly-L-glutamic acid of average molar mass in number (Mn) 5200 g/mol relative to a polyoxyethylene glycol (PEG) standard.

### Co-Polyamino Acid B3

The co-polyamino acid B3-1 (12.0 g) is solubilized in DMF (500 mL) by heating for 10 min at 40° C., and then are added successively and at room temperature N-methyl morpholine (NMM, 9.1 g, 90.3 mmol) and 2-hydroxypyridine N-oxide (HOPO, 3.0 g, 27.1 mmol). The reaction medium is then cooled to 0° C., then EDC.HCl (5.2 g, 27.1 mmol) is added and the medium is stirred for 1 h at 0° C. and then raised to room temperature. A solution of the molecule A1 (5.5 g, 13.5 mmol) and triethylamine (TEA, 1.9 mL, 13.5 mmol) in DMF (72 mL) is added, and the solution is stirred for 2 hours. The reaction medium is filtered through a 0.2 mm woven filter and poured drop by drop into 4.6 L of water containing 15% by weight of NaCl and HCl (pH 2) with stirring. At the end of the addition, the pH is readjusted to 2 with 37% HCl solution, and the suspension is left to stand overnight. The precipitate is collected by filtration and then rinsed with water (3×250 mL). The white solid obtained is solubilized in water (850 mL) by slowly adding an aqueous solution of 1N NaOH to pH 12 with stirring, and the solution is stirred for 45 min. The pH is adjusted to 7 with an aqueous solution of HCl, water (150 mL) and ethanol (580 mL) are added and the solution is filtered through an activated carbon disc R53SLP (3M). The solution obtain is filtered through a 0.2 μm PES filter and then purified by ultrafiltration against a 0.9% NaCl solution and then with water until the conductivity of the permeate is less than 50 μS/cm. The solution is filtered through a 0.2 μm filter and stored at 2-8° C.

Dry extract: 17.7 mg/g
DP (estimated from ¹H NMR): 39
From ¹H NMR: i=0.15
The calculated average molar mass of co-polyamino acid B3 is 7,870 g/mol.
Aqueous HPLC-SEC (PEG Calibrator): Mn=4,771 g/mol.

### Example B4

Co-Polyamino Acid B4: Sodium Poly-L-Glutamate Modified at its Two Ends by Molecule A4 and Having a Mean Number Average Molecular Mass (Mn) of 3,350 g/Mol
Co-Polyamino Acid B4-1: Poly-L-Benzyl Glutamate Resulting from the Polymerization of γ-Benzyl-L-Glutamate N-Carboxy Anhydride Initiated by Ethylenediamine.

In a previously oven-dried reactor, γ-benzyl-L-glutamate N-carboxy anhydride (500.0 g, 1.899 mmol) is solubilized in anhydrous DMF (1.12 L). The mixture is then stirred until complete dissolution, cooled to 0° C., and then ethylenediamine (4.76 g, 79.14 mmol) is introduced rapidly. After stirring for 24 h at 0° C., a solution of 4M HCl in dioxane (99 mL, 396 mmol) is added and then the reaction mixture is poured in over 30 min over a mixture of methanol (1.6 L) and PEI (6.3 L). After 16 h under agitation, the precipitate is filtered through a sintered filter, washed with PEI (2×1.12 L) and dried at 30° C. under reduced pressure.

### Co-Polyamino Acid B4

To a solution of molecule A4 (7.03 g, 9.07 mmol) in DMAc (40 mL) are successively added HOPO (1.11 g, 9.98 mmol), and EDC (2.26 g, 11.80 mmol).

To a solution of co-polyamino acid B4-1 (18.1 g) in DMAc (50 mL) at room temperature are successively added DIPEA (1.58 mL, 9.07 mmol) and then the previously prepared molecule A4 solution as described above.

After stirring for 24 h at room temperature, DMAc (200 mL) is added and the solution is placed at 60° C. under 6 bar of hydrogen in the presence of palladium on 5% alumina (4.6 g). After 24 h of reaction, the reaction medium is filtered through a sintered filter and then through an Omnipore 0.2 μm PTFE hydrophilic membrane.

The filtrate is then stirred, then are successively added acetone (1 V) and then a solution of sodium carbonate at 300 g/L (1 equivalent of Na₂CO₃ acid function) is added drop by drop. After 16 h, the precipitate is filtered through a sintered filter, washed with acetone (200 mL) and then dried at 30° C. under reduced pressure.

The white solid obtained is then solubilized in water (800 mL) then the pH is adjusted to 12 by adding 10 N aqueous sodium hydroxide solution. The mixture is stirred for 1 h before being neutralized to pH 7 by adding a 37% HCl solution. Ethanol (30% by weight) is added to the solution and the product is filtered on an activated R53SLP carbon disc (3M), and then through a 0.2 μm PES filter. The solution is then purified by ultrafiltration against a 0.9% NaCl solution, and then water until the conductimetry of the permeate is less than 50 μS/cm. The solution obtained is filtered through a 0.2 μm filter and stored at 2-8° C.

Dry extract: 23.2 mg/g
DP (estimated from ¹H NMR): 24
From ¹H NMR: i=0.080
The calculated average molar mass of co-polyamino acid B4 is 5,140 g/mol.
Organic HPLC-SEC (PEG Calibrator): Mn=3,350 g/mol.

### Example B5

Co-Polyamino Acid B5: Sodium Poly-L-Glutamate Modified at its Two Ends by Molecule A5 Whose Esters are Deprotected and Having a Mean Number Average Molecular Mass (Mn) of 3,700 g/Mol

### Co-Polyamino Acid B5-1: Poly-L-Benzyl Glutamate Modified at its Two Ends by the A5 Molecule.

To a molecule A5 solution (2.67 g, 1.43 mmol) in DMF (30 mL) at 0° C. is added HATU (0.54 g, 1.43 mmol) and DIPEA (0.503 g, 3.89 mmol). The solution is then introduced onto a solution of co-polyamino acid B4-1 (3.5 g) and triethylamine (TEA, 0.132 g, 1.30 mmol) in DMF (40 mL) at 0° C. and the medium is stirred for 18 h at a temperature of between 0° C. and room temperature. Dichloromethane (175 m1) is added and the organic phase is washed with an aqueous solution of 0.1 N HCl (3×90 m1), dried over Na₂SO₄, filtered and then poured over PEI (950 mL). The precipitate is sinter filtered, washed with PEI (2×100 mL) and dried at 30° C. under reduced pressure to give a poly-L-benzyl glutamate modified at both ends by the molecule A5.

### Co-Polyamino Acid B5-2: Poly-L-Benzyl Glutamate Modified at its Two Ends by the A5 Molecule Whose Esters are Deprotected

The co-polyamino acid B5-1 is solubilized in TFA (30 mL), and the solution is stirred for 2 h at room temperature and then is poured drop by drop over diisopropyl ether with stirring (300 mL). After 18 h, the white precipitate is recovered by filtration, triturated with PEI and dried under reduced pressure to give a poly-L-benzyl glutamate modified at both ends by the molecule A5 whose esters are deprotected.

### Co-Polyamino Acid B5

The co-polyamino acid B5-2 (1.97 g) is solubilized in DMAc (10 mL) and then hydrogenated (1 atm, 48 h, 65° C.) and purified according to a process similar to that used for the preparation of co-polyamino acid B4, but without the activated carbon disc filtration step. A sodium poly-L-glutamate modified at its two ends by the A5 molecule whose esters are deprotected

Dry extract: 13.2 mg/g
DP (estimated from ¹H NMR): 24
From ¹H NMR: i=0.072
The calculated average molar mass of co-polyamino acid B5 is 6,537 g/mol.
Organic HPLC-SEC (PEG Calibrator): Mn=3,700 g/mol.

### Example B6

Co-Polyamino Acid B6: Sodium Poly-L-Glutamate Modified at its Two Ends by Molecule A6 Whose Esters are Deprotected and Having a Mean Number Average Molecular Mass (Mn) of 5,000 g/Mol
Co-Polyamino Acid B6-1: Poly-L-Glutamic Acid Resulting from the Polymerization of γ-Benzyl-L-Glutamate N-Carboxy Anhydride Initiated by Hexylamine

In a jacketed reactor, γ-benzyl-L-glutamate N-carboxy anhydride (500 g, 1.90 mol) is solubilized in anhydrous DMF (1100 mL). The mixture is then stirred until complete dissolution is achieved, cooled to 0° C., and then hexylamine (6.27 mL, 47.5 mmol) is introduced rapidly. The mixture is stirred at 0° C. for 5 h, between 0° C. and 20° C. for 7 h and then at 20° C. for 7 h. The reaction medium is then heated at 65° C. for 2 h, cooled to 55° C. and methanol (3300 mL) is introduced in 1 h 30. The reaction mixture is then cooled to 0° C. and left under agitation for 18 h. The white precipitate is recovered by filtration, washed with diisopropyl ether (2×800 mL) and then dried under reduced pressure at 30° C. to give a poly(gamma-benzyl-L-glutamic) acid (PBLG).

To a PBLG solution (180 g) in N,N-dimethylacetamide (DMAc, 450 mL) is added Pd/Al₂O₃ (36 g) under an argon atmosphere. The mixture is placed in a hydrogen atmosphere (10 bar) and stirred at 60° C. for 24 h. After cooling to room temperature and filtration of the catalyst on a sintered P4 then through an Omnipore 0.2 μm PTFE hydrophilic membrane, a solution of water at pH 2 (2700 mL) is poured drop by drop on the solution of DMAc, over a period of 45 minutes with stirring. After 18 h with stirring, the white precipitate is recovered by filtration, washed with water (4×225 mL) and then dried under reduced pressure at 30° C.

### Co-Polyamino Acid B6

By a coupling method similar to that used for the preparation of co-polyamino acid B3 applied to molecule A6 (31.06 g, 42.08 mmol) and co-polyamino acid B6-1 (36.80 g), a beige solid is obtained after the acid precipitation step. This solid is diluted in TFA (100 g/L) and the mixture is stirred at room temperature for 3 h. The solution is then poured drop by drop into the water (3 V) with stirring. After stirring for 16 h, the precipitate is recovered by filtration and then washed with water. The solid obtained is then solubilized in water by adjusting the pH to 7 by adding 10 N aqueous sodium hydroxide solution. Once the solubilization is complete, the pH is adjusted to pH 12 for 1 h by adding a 1N NaOH solution. After neutralization to pH 7 by the addition of a solution of 1N HCl, the product is purified by a method similar to that used for the preparation of co-polyamino acid B3 (carbofiltration and ultrafiltration). A sodium poly-L-glutamate modified with the A6 molecule whose esters are deprotected is obtained.

Dry extract: 28.2 mg/g
DP (estimated from ¹H NMR): 40
From ¹H NMR: i=0.15
The calculated average molar mass of co-polyamino acid B6 is 9,884 g/mol.
Organic HPLC-SEC (PEG Calibrator): Mn=5,000 g/mol.

### Part C—Fast-Acting and Slow-Acting Insulin Solutions

### Example C1: Fast-Acting Insulin Analogue Lispro Solution at 600 U/mL

This solution is an insulin solution prepared from a lispro insulin powder. This product is a fast-acting insulin analogue. The excipients used are m-cresol, and/or phenol, glycerol, zinc chloride, sodium hydroxide and hydrochloric acid for pH adjustment (pH 7-7.8) and water. The zinc chloride concentration is 1800 μm and that of the glycerol is 230 mM. The m-cresol and phenol concentrations vary according to the concentrations desired in the final CB1 and CB2 preparations.

### Example C2: Insulin Glargine Solution at 100-325 U/mL

This solution is a glargine insulin solution prepared from a glargine insulin powder. This product is a slow-acting insulin analogue. The excipients used are zinc chloride, m-cresol, and/or phenol, glycerol, sodium hydroxide and hydrochloric acid for pH adjustment (pH 4) and water. The zinc concentration is 4.5 μM per 1 U/ml of insulin. The concentrations of glycerol and the phenolic excipients, m-cresol and phenol vary according to the concentrations desired in the final CB1 and CB2 preparations.

### Example C3: Insulin Glargine Solution at 100-325 IU/mL

This solution is a glargine insulin solution prepared from a glargine insulin powder. This product is a slow-acting insulin analogue. The excipients used are zinc chloride, m-cresol, glycerol, sodium hydroxide and hydrochloric acid for pH adjustment (pH 4) and water. The zinc concentration is 2 μM per 1 IU/mL of insulin. The concentrations of glycerol and the phenolic excipients, m-cresol and phenol vary according to the concentrations desired in the final CB1 and CB2 preparations.

### Part CA—Compositions Comprising Insulin Glargine

### CA1 Method of Preparation: Preparation of a Concentrated Composition of Co-Polyamino Acid/Insulin Glargine at pH 7.2, According to a Method Using Insulin Glargine in Liquid Form (in Solution) and a Co-Polyamino Acid in Liquid Form (in Solution).

To a stock solution of co-polyamino acid at pH 7.1 are added concentrated solutions of NaCl and zinc chloride so as to reach the concentrations targeted in the final composition. To this solution of co-polyamino acid is added a solution of insulin glargine as described in Example C2. A turbidity appears. The pH is adjusted to pH 7.5 by adding concentrated NaOH and the solution is placed under static conditions at +40° C. until complete solubilization is achieved. The resulting solution is visually clear and is allowed to cool to 20-25° C. The pH is adjusted to 7.2 by adding a solution of hydrochloric acid.

### CA2 Method of Preparation: Preparation of a Concentrated Composition of Co-Polyamino Acid/Insulin Glargine at pH 7.2, According to a Method Using Insulin Glargine in Liquid Form (in Solution) and a Co-Polyamino Acid in Liquid Form (in Solution).

To this stock solution of co-polyamino acid at pH 7.1 is added a solution of insulin glargine as described in Example C3. A turbidity appears. The pH is adjusted to pH 8.5 by the addition of concentrated NaOH. The solution obtained is visually clear after 30 minutes at 20-25° C. A concentrated solution of zinc chloride is added to obtain the targeted concentration in the final CB2 composition. The pH is adjusted to 7.2 by adding a solution of hydrochloric acid.

According to the CA1 or CA2 preparation methods, co-polyamino acid/insulin glargine compositions have been prepared with insulin glargine concentrations from 50 U/mL to 200 U/mL.

### CB Part—Compositions Comprising Insulin Glargine and Insulin Lispro

### CB1 Method of Preparation: Preparation of a Co-Polyamino Acid/Insulin Glargine/Insulin Lispro Composition at pH 7.2

To the concentrated co-polyamino acid/insulin glargine composition at pH 7.2 described in Example CA1 is added a solution of lispro as described in Example C1 and if necessary, water. The solution obtained is clear and contains the desired concentrations of co-polyamino acid, zinc, glycerol, NaCl, m-cresol and/or phenol.

If necessary, the pH is adjusted to the target of 7.2 by adding hydrochloric acid or sodium hydroxide solutions.

The compositions are filtered (0.22 μm) and stored at 4° C.

### CB2 Method of Preparation: Preparation of a Co-Polyamino Acid/Insulin Glargine/Insulin Lispro Composition at pH 7.2

To the concentrated co-polyamino acid/insulin glargine composition at pH 7.2 described in Example CA2 is added a solution of lispro as described in Example C1 and if necessary, water. The solution obtained is clear and contains the desired concentrations of co-polyamino acid, zinc, glycerol, NaCl, m-cresol and/or phenol.

If necessary, the pH is adjusted to the target of 7.2 by adding hydrochloric acid or sodium hydroxide solutions.

The compositions are filtered (0.22 μm) and stored at 4° C.

### Example CB1: Co-Polyamino Acid/Insulin Glargine/Insulin Lispro Compositions at pH 7.2

According to the CB1 and CB2 preparation methods, co-polyamino acid/insulin glargine/insulin lispro compositions were prepared with insulin glargine concentrations of 75 or 150 U/mL and insulin lispro of 25 or 75 U/mL. The solutions contain 230 mM of glycerin and various concentrations of sodium chloride, zinc chloride and phenolic ligands. These compositions are described in Table 1.

TABLE 1
Compositions of insulin glargine and insulin lispro in the presence of co-
polyamino acids.

| | Co- | Concentration | Insulin | | | | | | Visual |
|---|---|---|---|---|---|---|---|---|---|
| | polyamino | in Co- | glargine | Insulin | | [phenol] | | | appearance |
| Composition | acid | polyamino acid | (UI/mL) | Lispro | [m-cresol] | (mM) | [ZnCl₂] | [NaCl] | of the solution |
| CB1-1 | B1 | 2.6 | 150 | 50 | 35 | — | 1 | — | Clear |
| CB2-1 | B1 | 2.6 | 150 | 50 | 28 | — | 1 | — | Clear |
| CB2-2 | B1 | 2.6 | 150 | 50 | 25 | — | 1 | — | Clear |
| CB1-2 | B1 | 2.6 | 150 | 50 | 16 | 16 | 1 | — | Clear |
| CB1-3 | B1 | 2.6 | 150 | 50 | 19 | 19 | 1 | — | Clear |
| CB1-4 | B1 | 2.6 | 150 | 50 | 19 | 10 | 1 | — | Clear |
| CB1-5 | B1 | 2.2 | 150 | 50 | 35 | — | 1 | — | Clear |
| CB2-3 | B1 | 2.2 | 150 | 50 | 25 | — | 1 | — | Clear |
| CB1-6 | B1 | 1.3 | 75 | 25 | 35 | — | 0.5 | — | Clear |
| CB1-7 | B1 | 1.3 | 75 | 25 | 28 | — | 0.5 | — | Clear |
| CB1-8 | B1 | 1.3 | 75 | 25 | 16 | 16 | 0.5 | — | Clear |
| CB1-9 | B2 | 3 | 150 | 50 | 35 | — | 1 | — | Clear |
| CB1-10 | B2 | 3 | 150 | 50 | 28 | — | 1 | — | Clear |
| CB1-11 | B2 | 1.5 | 75 | 25 | 28 | — | 0.5 | — | Clear |
| CB1-12 | B2 | 1.2 | 75 | 25 | 28 | — | 0.5 | 5 | Clear |
| CB1-13 | B2 | 1.35 | 75 | 25 | 16 | 16 | 0.5 | — | Clear |
| CB1-14 | B2 | 1.35 | 75 | 25 | 19 | 19 | 0.5 | — | Clear |
| CB1-15 | B2 | 0.95 | 75 | 25 | 16 | 16 | 0.5 | 5 | Clear |
| CB1-16 | B2 | 0.95 | 75 | 25 | 19 | 19 | 0.5 | 5 | Clear |
| CB1-17 | B4 | 3.1 | 150 | 50 | 35 | — | 1.4 | 10 | Clear |
| CB1-18 | B4 | 3 | 150 | 50 | 28 | — | 1.4 | 10 | Clear |
| CB1-19 | B4 | 1.5 | 75 | 25 | 28 | — | 0.6 | 5 | Clear |
| CB1-20 | B4 | 1.3 | 75 | 25 | 19 | 19 | 0.6 | 5 | Clear |
| CB1-21 | B3 | 2.2 | 75 | 25 | 28 | — | | | Clear |
| CB1-22 | B3 | 1.8 | 75 | 25 | 28 | — | | 10 | Clear |
| CB1-23 | B3 | 2 | 75 | 25 | 19 | 19 | 0.5 | | Clear |
| CB1-24 | B3 | 1.5 | 75 | 25 | 19 | 19 | 0.5 | 5 | Clear |
| CB1-25 | B5 | 1.3 | 75 | 25 | 28 | — | 0.5 | — | Clear |
| CB1-26 | B5 | 1.15 | 75 | 25 | 28 | — | 0.5 | 5 | Clear |
| CB1-27 | B5 | 1.15 | 75 | 25 | 19 | 19 | 0.5 | — | Clear |
| CB1-28 | B5 | 0.95 | 75 | 25 | 19 | 19 | 0.5 | 5 | Clear |

### Part D—Results

### Part DA: Demonstration of the Solubilization of Glargine at pH 7.1 by the Compositions According to the Invention

### Protocol DA1: Determination of the Minimum Concentration of Co-Polyamino Acid to Solubilize Insulin Glargine at pH 7.1.

To a stock solution of co-polyamino acid at pH 7 are added concentrated solutions of m-cresol, glycerin, NaCl and zinc chloride. The amount of excipients added is adjusted so as to obtain the target compositions in co-polyamino acid/insulin glargine 50 U/mL at pH 7.1.

In a 3 mL vial, 0.5 mL of a solution of insulin glargine at a concentration of 100 U/mL, prepared according to Example C2, is added to a volume of 0.5 mL of the co-polyamino acid solution and excipients to obtain 50 U/mL of a co-polyamino acid (mg/mL)/insulin glargine composition at pH 7.1. A turbidity appears. The pH is adjusted to pH 7.1 by adding concentrated NaOH and the solution is placed in static in an oven at 40° C. for 1 night. This operation is perform for different concentrations of the co-polyamino acid. After overnight at 40° C., the samples are visually inspected and subjected to a light scattering measurement under static conditions at a 173° angle using a Zetasizer (Malvern). The minimum concentration of co-polyamino acid that make it possible to solubilize glargine is defined as the lowest concentration for which the co-polyamino acid/insulin glargine mixture at pH 7.1 is visually clear and has a scattered intensity of less than 1000 kcps/s (kilo shots per second).

### Example DA1: Solubilization of Insulin Glargine at pH 7.1

According to the protocol DA1, solutions of co-polyamino acid and insulin glargine at pH 7.1 were prepared by varying the concentration of the co-polyamino acid. These solutions contain 184 mM glycerin and compositions and variable contents of phenolic ligand(s) and of sodium chloride. The minimum concentration of co-polyamino acid for solubilizing glargine is between a concentration value for which the scattered intensity is less than 1000 kcps and a concentration value for which the scattered intensity is greater than 1000 kcps. The results are given in Table 2.

TABLE 2
Minimum concentration of co-polyamino acid to solubilize 50 IU/mL insulin
glargine at pH 7.1

| | Co-poly- | [m-cresol] | [phenol] | [ZnCl₂] | [NaCl] | Minimum concentration |
|---|---|---|---|---|---|---|
| Composition | amino acid | (mM) | (mM) | (mM) | (mM) | in co-polyamino acid (mg/ml) |
| DA-1 | B2 | 35 | — | 0.23 | — | 0.8 < [Cmin] ≤ 0.85 |
| DA-3 | B2 | 28 | — | 0.23 | 5 | 0.65 < [Cmin] ≤ 0.7 |
| DA-4 | B2 | 28 | — | 0.23 | 7.5 | 0.55 < [Cmin] ≤ 0.6 |
| DA-6 | B2 | 19 | 19 | 0.23 | 0 | 0.7 < [Cmin] ≤ 0.75 |
| DA-7 | B2 | 19 | 19 | 0.23 | 5 | 0.55 < [Cmin] ≤ 0.6 |
| DA-8 | B2 | 16 | 16 | 0.23 | 0 | 0.75 < [Cmin] ≤ 0.8 |
| DA-9 | B2 | 16 | 16 | 0.23 | 5 | 0.5 < [Cmin] ≤ 0.55 |
| DA-10 | B5 | 35 | — | 0.23 | — | 0.65 < [Cmin] ≤ 0.7 |
| DA-12 | B5 | 28 | — | 0.23 | 5 | 0.6 < [Cmin] ≤ 0.65 |
| DA-13 | B5 | 19 | 19 | 0.23 | — | 0.6 < [Cmin] ≤ 0.65 |
| DA-14 | B5 | 19 | 19 | 0.23 | 5 | 0.5 < [Cmin] ≤ 0.55 |
| DA-15 | B3 | 35 | — | 0.23 | — | 1.15 < [Cmin] ≤ 1.2 |
| DA-17 | B3 | 28 | — | 0.23 | 5 | 1.05 < [Cmin] ≤ 1.1 |
| DA-19 | B3 | 19 | 19 | 0.23 | 5 | 0.85 < [Cmin] ≤ 0.9 |

Compositions that show a decrease in the m-cresol concentration exhibit an improved minimum solubilization concentrations.

### Part DB: Demonstration of the Physical Stability of the Compositions According to the Invention by Studying the Previously Prepared Compositions

### Protocol DB1: Study of the Physical Stability of the Co-Polyamino Acid Insulin Glargine/Insulin Lispro Compositions at pH 7.2.

At least five 3 mL glass cartridges filled with 1 mL of a co-polyamino acid/insulin glargine/prandial insulin composition are placed in an oven at 30° C. under static conditions. The cartridges are inspected visually at a bimonthly frequency to detect the appearance of visible particles or turbidity. This inspection is carried out according to the recommendations of the European Pharmacopoeia (EP 2.9.20): the cartridges are subjected to illumination of at least 2000 Lux and are observed in front of a white background and a black background. The number of weeks of stability corresponds to the time from which the majority of the cartridges has visible particles or is turbid. The results are described in Table 3.

TABLE 3
Physical stability of the compositions of the invention.

| | | Concentration in | Insulin | Insulin | | | | | Stability at 30° C. |
|---|---|---|---|---|---|---|---|---|---|
| | Co-poly- | Co-polyamino acid | glargine | Lispro | [m-cresol] | [phenol] | [ZnCl₂] | [NaCl] | (Number of |
| Composition | amino acid | (mg/mL) | (UI/mL) | (UI/mL) | (mM) | (mM) | (mM) | (mM) | weeks) |
| CB1-1 | B1 | 2.6 | 150 | 50 | 35 | — | 1 | — | >15 |
| CB2-2 | B1 | 2.6 | 150 | 50 | 25 | — | 1 | — | >15 |
| CB1-2 | B1 | 2.6 | 150 | 50 | 16 | 16 | 1 | — | >15 |
| CB1-3 | B1 | 2.6 | 150 | 50 | 19 | 19 | 1 | — | >15 |
| CB1-4 | B1 | 2.6 | 150 | 50 | 19 | 10 | 1 | — | >15 |
| CB1-5 | B1 | 2.2 | 150 | 50 | 35 | — | 1 | — | >14 |
| CB2-3 | B1 | 2.2 | 150 | 50 | 25 | — | 1 | — | >15 |
| CB1-6 | B1 | 1.3 | 75 | 25 | 35 | — | 0.5 | — | >15 |
| CB1-8 | B1 | 1.3 | 75 | 25 | 16 | 16 | 0.5 | — | >15 |
| CB1-9 | B2 | 3 | 150 | 50 | 35 | — | 1 | — | >6 |
| CB1-10 | B2 | 3 | 150 | 50 | 28 | — | 1 | — | >10 |
| CB1-11 | B2 | 1.5 | 75 | 25 | 28 | — | 0.5 | — | >6 |

The physical stability of the exemplified compositions remains at an excellent level.

### D Pharmacokinetic

D1: Protocol for measuring the pharmacokinetics of insulin glargine and insulin lispro formulations.

Studies in pigs have been carried out in order to evaluate the pharmacokinetics of insulins after administration of the CB1-5 and CB2-3 compositions.

The pharmacokinetic profiles of basal insulin (sum of the circulating concentration of insulin glargine and its main metabolite M1) from the CB1-5 and CB2-3 compositions were observed in pigs in 2 simultaneous sessions.

Fourteen animals that were fasted for approximately 2.5 hours were injected subcutaneously in the flank at a dose of 0.5 U/kg insulin. Blood samples were taken during the 22 h following administration to describe the pharmacokinetics of the basal insulin. The levels of glargine, glargine-M1 were determined by a specific bioanalysis method.

The pharmacokinetic parameters of basal insulin are presented in Table 4.

The pharmacokinetic parameters determined are as follows:
AUC₁₃₋₂₂ₕ, AUC₁₆₋₂₂ₕ correspond to the area under the curve of the concentrations of insulin glargine (and its metabolite M1) as a function of time from 13 to 22 h respectively and 16 and 22 h post-administration;
AUCₗₐₛₜ corresponds to the area under the curve of insulin glargine concentrations (and its metabolite M1) as a function of time between time 0 and the last time measurement performed on the subject.

The results obtained show that, while the AUCₗₐₛₜ was preserved between the two formulations (a difference of less than 1% relative to the CB1-5 formulation), the partial terminal AUCs increased when the quantity of m-cresol in the formulation decreased (AUC₁₃₋₂₂ₕ and AUC₁₆₋₂₂ₕ, decrease of about 25% compared to the CB1-5 formulation).

TABLE 4
mean pharmacokinetic parameters (ratio of means)
of compositions CB1-5 and CB2-3 comprising
co-polyamino acid B1/insulin glargine 150 U/mL/insulin lispro
50 U/mL and respectively 35 mM and 25 mM of m-cresol.

| | AUCₗₐₛₜ | AUC₁₃₋₂₂ₕ | AUC₁₆₋₂₂ₕ |
|---|---|---|---|
| | (h · pmol/L) | (h · pmol/L) | (h · pmol/L) |
| CB1-5 | 2404 (29) | 388 (63) | 209 (81) |
| CB2-3 | 2381 (28) | 484 (52) | 261 (62) |
| Delta | −0.9 | −24.6 | −24.8 |
| (% relative to CB2-3) | | | |

1. A composition in the form of an injectable aqueous solution, whose pH is from 6.0 to 8.0, comprising at least:
a) a basal insulin whose isoelectric point (pI) is from 5.8 to 8.5,
b) m-cresol in a concentration lower than or equal to 30 mM (0<[m-cresol]≤30 mM), and
c) a co-polyamino acid bearing carboxylate charges and hydrophobic radicals Hy, said co-polyamino acid consisting of glutamic or aspartic units and said hydrophobic radicals Hy being according to the following Formula X: 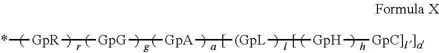
in which
GpR is chosen among the radicals according to formulas VII, VII′ or VII″: 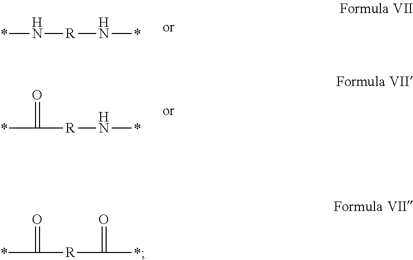
GpG and GpH, which are identical or different, are chosen among the radicals according to formulas XI or XI′: 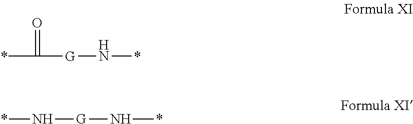
GpA is chosen among the radicals according to formula VIII 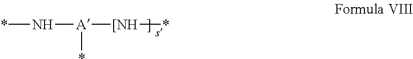
In which A′ is chosen among the radicals according to formulas VIII′, VIII″ or VIII′″ 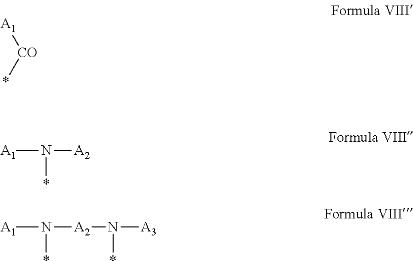
GpL is chosen among the radicals according to formula XII 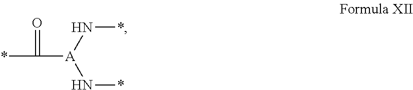
GpC is a radical according to formula IX: 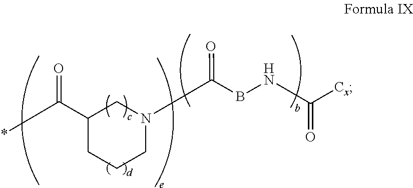
the * indicates the attachment sites of the different groups bound by amide functions;
a is an integer equal to 0 or 1 and a′=1 if a=0 and a′=1, 2 or 3 if a=1;
a′ is an integer equal to 1, to 2 or 3
b is an integer equal to 0 or 1;
c is an integer equal to 0 or 1, and if c is equal to 0 then d is equal to 1 or 2;
d is an integer equal to 0, 1 or 2;
e is an integer equal to 0 or 1;
g is an integer equal to 0, 1, 2, 3, 4, 5 or 6;
h is an integer equal to 0, 1, 2, 3, 4, 5 or 6,
l is an integer equal to 0 or 1 and l′=1 if l=0 and l′=2 if l=1;
r is an integer equal to 0, 1 or 2, and
s′ is an integer equal to 0 or 1, and
A, A₁, A₂ and A₃, which are identical or different, are linear or branched alkyl radicals, and/or substituted by a radical from a saturated, unsaturated or aromatic ring, comprising from 1 to 8 carbon atoms;
B is a linear or branched alkyl radical, and/or comprising an aromatic ring comprising from 1 to 9 carbon atoms or an unsubstituted ether or polyether radical comprising from 4 to 14 carbon atoms and from 1 to 5 oxygen atoms;
Cₓ is a linear or branched monovalent alkyl radical, and/or comprising a cyclic part, in which x indicates the number of carbon atoms and:
When the hydrophobic radical -Hy bears 1 -GpC, then 9≤x≤25,
When the hydrophobic radical -Hy bears 2 -GpC, then 9≤x≤15,
When the hydrophobic radical -Hy bears 3 -GpC, then 7≤x≤13,
When the hydrophobic radical -Hy bears 4 -GpC, then 7≤x≤11,
When the hydrophobic radical -Hy bears at least 5 -GpC, then 6≤x≤11,
G is a branched alkyl radical of 1 to 8 carbon atoms, said alkyl radical bearing one or more free carboxylic acid function(s)
R is a radical chosen in the group consisting of a linear or branched divalent alkyl radical comprising from 1 to 12 carbon atoms, a linear or branched divalent alkyl radical comprising from 1 to 12 carbon atoms bearing one or more —CONH₂ functions or an unsubstituted ether or polyether radical comprising from 4 to 14 carbon atoms and from 1 to 5 oxygen atoms:
the hydrophobic radical(s) -Hy according to formula X being bound to PLG:
via a covalent bond between a carbonyl of the hydrophobic radical -Hy and a nitrogen atom borne by the PLG thus forming an amide function resulting from the reaction of an amine function borne by the PLG and an acid function borne by the precursor -Hy′ of the hydrophobic radical -Hy, and
via a covalent bond between a nitrogen atom from the hydrophobic radical -Hy and a carbonyl borne by the PLG thus forming an amide function resulting from the reaction of an amine function of the precursor -Hy′ of the hydrophobic radical -Hy and an acid function borne by the PLG,
the ratio M between the number of hydrophobic radicals and the number of glutamic or aspartic units being between 0<M≤0.5;
when several hydrophobic radicals are borne by a co-polyamino acid then they are identical or different,
the degree of polymerization DP in glutamic or aspartic units for the PLG chains is from 5 to 250;
the free carboxylic acid functions being in the form of an alkali metal salt chosen in the group consisting of Na⁺ and K⁺.
